(19) United States
(12) Patent Application Publication
(10) Pub. No.: US 2023/0322866 A1
(43) Pub. Date: Oct. 12, 2023

(54) PHOSPHATE-REGULATED EXPRESSION OF BIOLOGICALLY ACTIVE RECOMBINANT CORONAVIRUS GLYCOPROTEINS AND OTHER RECOMBINANT PROTEINS IN PHAEODACTYLUM TRICORNUTUM

(71) Applicant: Suncor Energy Inc., Calgary (CA)

(72) Inventors: Samuel S. Slattery, London (CA); Daniel J. Giguere, London (CA); Martin Flatley, Sarnia (CA); Gregory B. Gloor, London (CA); David R. Edgell, London (CA)

(21) Appl. No.: 17/898,870

(22) Filed: Aug. 30, 2022

(30) Foreign Application Priority Data
Aug. 30, 2021 (CA) 3129298
Nov. 5, 2021 (CA) 3137825

(51) Int. Cl. C12N 15/79; A61K 39/215; C07K 14/165; G01N 33/543; C12N 1/12

(52) U.S. Cl. CPC C07K 14/165; A61K 39/215; C12N 1/12; C12N 15/79; G01N 33/54388; C12N 2500/24; C12N 2500/42

(57) ABSTRACT

Phosphate-regulated expression of recombinant glycoprotein antigens and other recombinant proteins in diatoms is described herein. More specifically, described herein is the expression and purification of glycosylated, immunogenic, and serologically active receptor-binding domain (RBD) of the SARS-CoV-2 spike protein, as well as SARS-CoV-2 nucleocapsid protein, in the marine pennate diatom Phaeodactylum tricornutum, as well as a functional lateral flow assay-based diagnostic device based on the produced recombinant RBD and nucleocapsid protein. Also described herein is the use of phosphate/iron levels in culture media to regulate expression/secretion of recombinant proteins under control of an HASP1 promoter in P. tricornutum or other suitable host cells. Also described herein is a method for increasing the expression/secretion of a recombinant protein by engineering the recombinant protein to lack a Tobacco Etch Virus (TEV) protease cleavage site.

The present description relates to the expression of recombinant glycoprotein antigens and other recombinant proteins in the diatom Phaeodactylum tricornutum. More specifically, the present description relates to the expression of recombinant glycoproteins comprising coronavirus polypeptide antigens having complex N-linked glycosylation that are sufficiently natively glycosylated, immunogenic, and serologically active. The present description also relates to the use of phosphate/iron levels in culture media to regulate the expression/secretion of recombinant proteins under control of a diatom HASP1 promoter in P. tricornutum.

### BACKGROUND

The COVID-19 pandemic caused by the severe acute respiratory syndrome coronavirus-2 (SARS-CoV-2, a betacoronavirus) has exposed weaknesses in the ability of the biomedical community to respond to this and future pandemics. In particular, a major barrier to controlling the spread of the disease has been the availability of cheap, reliable and serologically reactive sources of SARS-CoV-2 proteins for use in diagnostic testing. Typical protein overproduction systems include bacteria, yeast, mammalian cell lines and several plant species. A major drawback of bacteria and yeast expression systems is that they lack the proper glycosylation machinery to produce recombinant proteins having the complex glycosylation patterns similar to those found on human proteins. This represents an especially important drawback for expressing recombinant coronavirus antigenic proteins, which have been shown to be extensively glycosylated (Grant et al., 2020; Walls et al., 2016). Conversely, while recombinant proteins produced in mammalian cell lines typically possess the necessary glycosylation machinery, such expression systems are costly to operate, requiring specialized infrastructure and biocontainment. While there have been several attempts to genetically engineer yeast and other non-mammalian species to be versatile platforms capable of producing a variety of therapeutic recombinant proteins having human-like glycosylation, those efforts have largely proved unsuccessful, with the few successes being limited to individual strains being adapted to produce individual proteins with their activities being individually validated. Thus, improved methods for producing cost-effective recombinant coronavirus proteins that are sufficiently natively glycosylated, immunogenic, and serologically active would be highly desirable.

### SUMMARY

In a first aspect, described herein is a recombinant glycoprotein or protein comprising a coronavirus polypeptide antigen having a glycosylation or other post-translational modification pattern (e.g., N-linked glycosylation pattern and/or phosphorylation pattern) produced by, or characteristic of, post-translational modification by Phaeodactylum tricornutum. In some implementations, the coronavirus polypeptide antigen may be a betacoronavirus polypeptide antigen (e.g., SARS-CoV-2, SARS-CoV, or MERS-CoV) and the polypeptide antigen may be from a surface glycoprotein or protein (e.g., spike (S) protein, a nucleocapsid (N) protein, a membrane protein, or an envelope protein), or a fragment thereof, such as a coronavirus spike protein’s receptor binding domain (RBD). In some implementations, the polypeptide antigen is biologically active and cross-reacts with antibodies raised against the corresponding native viral proteins.

In a further aspect, described herein is an immunogenic composition (e.g., vaccine) comprising a recombinant protein as described herein, and a suitable adjuvant.

In a further aspect, described herein is a P. tricornutum host cell that produces a recombinant glycoprotein or protein as described herein, wherein the host cell comprises an exogenous expression cassette encoding the recombinant glycoprotein or protein operably linked to a promoter (e.g., an HASP1 promoter).

In a further aspect, described herein is a diagnostic device (e.g., a lateral flow test) comprising a recombinant glycoprotein or protein as described for use in detecting the presence and/or concentration of antibodies that bind to the recombinant protein.

In a further aspect, described herein is an antigen-antibody complex comprising a recombinant glycoprotein or protein as described herein and an anti-coronavirus antibody that cross-reacts therewith, wherein the antibody is from a biological sample from a subject.

In a further aspect, described herein is a method for triggering the production of antibodies against a coronavirus polypeptide antigen, the method comprising administering to a subject the immunogenic composition as described herein.

In a further aspect, described herein is a method for detecting antibodies specific to a coronavirus polypeptide antigen in a biological sample, the method comprising: (a) contacting the biological sample with a recombinant glycoprotein or protein as described herein; and (b) detecting a complex formed between antibodies specific to the coronavirus polypeptide antigen and the recombinant glycoprotein.

In a further aspect, described herein is a polynucleotide encoding a recombinant glycoprotein or protein as described herein, such as a codon-optimized polynucleotide for increased expression in P. tricornutum host cells, relative to a polynucleotide encoding a corresponding protein using unoptimized or native codons.

In a further aspect, described herein is an expression cassette or expression vector (e.g., plasmid) comprising a polynucleotide as described herein operably linked to a promoter, wherein the promoter is heterologous with respect to the polynucleotide.

In a further aspect, described herein is a P. tricornutum chromosome comprising an expression cassette as described herein.

In a further aspect, described herein is a P. tricornutum host cell that produces a recombinant protein or glycoprotein as described herein, wherein the host cell comprises an expression cassette as described herein or a P. tricornutum chromosome as described herein.

In a further aspect, described herein is a method for producing a recombinant protein, the method comprising: (a) providing P. tricornutum host cells comprising a polynucleotide encoding the recombinant protein in an expression cassette under control of an HASP1 (highly abundant secreted protein 1) promoter; and (b) culturing the host cells in a production medium for a sufficient period of time to induce expression of the recombinant protein, the production medium being maintained an inorganic phosphate concentration sufficiently low such that the recombinant protein is expressed at a level higher than when the host cells are cultured under corresponding conditions in a phosphate-replete medium.

### General Definitions

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented.

The use of the word “a” or “an” when used in conjunction with the term “comprising” in the claims and/or the specification may mean “one” but it is also consistent with the meaning of “one or more”, “at least one”, and “one or more than one”.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “about” is used to indicate that a value includes the standard deviation of error for the device or method being employed in order to determine the value. In general, the terminology “about” is meant to designate a possible variation of up to 10%. Therefore, a variation of 1, 2, 3, 4, 5, 6, 7, 8, 9 and 10% of a value is included in the term “about”. Unless indicated otherwise, use of the term “about” before a range applies to both ends of the range.

As used herein, the expressions “glycoprotein” and “protein” may be used interchangeably depending on the type of post-translation modification(s) present on the expressed polypeptide (which may vary depending, for example, on the host organism or microorganism). In general, as used herein, “recombinant glycoprotein” refers to an expressed protein that comprises complex glycosylation patterns typical of those added during expression in eukaryotic cells, whereas the expression “recombinant protein” refers to an expressed protein that may or may not comprise any post-translational modification.

Other objects, advantages and features of the present description will become more apparent upon reading of the following non-restrictive description of specific embodiments thereof, given by way of example only with reference to the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the appended drawings:

FIG. 1 shows a list of plasmids used in this study.

FIG. 2 is a map of the plasmid vector used to express the SARS-CoV-2 protein antigens in P. tricornutum.

FIGS. 3A and 3B shows diagnostic PCRs performed on individual P. tricornutum clones for the RBD coding region to evaluate stable maintenance of the plasmid expressing PtRBD in both the wild-type (FIG. 3A) and histidine auxotroph (FIG. 3B) strains of P. tricornutum. The expected product is between 750 and 1,000 bp. PtRBD, codon-optimized for P. tricornutum; HsRBD, codon-optimized for HEK293 cells; M, 1-kb ladder.

FIGS. 4A and 4B shows the PtRBD protein expression. FIG. 4A shows a Coomassie-stained gel of whole cell lysates of 3 histidine auxotroph clones harbouring pSS2 or pSS7. M, prestained ladder. FIG. 4B shows a Western blot of whole cell lysates from FIG. 4A with a polyclonal anti-RBD antibody.

FIG. 5 shows the results of whole cell extracts separated by SDS-PAGE relating to nine different fermentation conditions, with samples taken at different P. tricornutum growth phases (“ML”: mid-log; “S” Stationary; “LS”: late-stationary. Upper panels are Coomassie-stained gels and lower panels are Western blots with anti-RBD polyclonal antibody. HEK293-RBD was used as positive control for Western blots.

FIGS. 6A, 6B, 6C, 6D, and 6E shows a phosphate-regulated expression of eGFP from the HASP1 promoter. FIG. 6A shows a schematic of eGFP expression plasmid with the putative phosphate-regulatory motifs (P1BS-like) in the HASP1 promoter region indicated below. FIG. 6B shows a Western blot of whole cell extracts from indicated growth conditions using an anti-GFP antibody. The (+) control lane is purchased 6X-histidine tagged GFP. eGFP expressed in our experiments is not 6X-histidine tagged. FIG. 6C shows a plot of eGFP extracellular secretion over time under different media conditions for one clone of pSS10. FIG. 6D shows a plot of the growth of eGFP expressing strain over time in different media conditions. FIG. 6E shows a plot of eGFP extracellular secretion over time under different media conditions for six clones of pSS10 (c1-c6) in 5% phosphate 5% iron or modified L1 media. For FIGS. 6C-6E, supernatant fluorescence values were subtracted from values taken from supernatants of wild-type P. tricornutum grown in parallel to correct for autofluorescence

FIGS. 7A, 7B, and 7C shows a phosphate-regulated expression of algae codon-optimized RBD from the HASP1 promoter. FIG. 7A shows RBD expression in response to varying phosphate concentrations measured over time. Shown is a Coomassie-stained gel (top) of whole cell extracts from cultures harbouring pSS2 at day 0, 3 and 5 post inoculation into media with 0%, 1%, 10% or 100% of phosphate levels as compared to our full L1 media. RBD expression was assessed by Western blotting (bottom) using a polyclonal anti-RBD antibody. The positive control (+) is 5 ng of commercially available RBD purified from HEK293 cells. FIG. 7B shows RBD expression in a 5-L bioreactor. The top image is a Coomassie-stained gel of whole cell lysates sampled at the indicated day, while the bottom image is a Western blot using a polyclonal anti-RBD antibody. FIG. 7C shows a plot of phosphate levels (filled circles), iron levels (filled diamonds), and growth (open circles). Phosphate and iron are plotted as parts per million (ppm). Growth is plotted as absorbance readings at A₆₇₀ nM.

FIGS. 8A, 8B, 8C, 8D, 8E, and 8F shows purification and activity of the PtRBD and the effect of the TEV protease cleavage site. FIG. 8A shows representative gel images of individual chromatographic steps in the purification, starting with the HisPrep FF column on the left. For each step, numbers above the gel indicate elution fractions. FIG. 8B shows a Western blot of column flow throughs of RBD-6His containing the TEV protease site. FIG. 8C shows a Western blot of column flow throughs of RBD-6His lacking the TEV protease site. On the blots of FIG. 8B and FIG. 8C, “L” represents lysates, “F” represents flow through, and “E” represents eluates. FIGS. 8D and 8E show the secretion of the algae-RBD. FIG. 8D is representative gel image of concentrated cell-free supernatant from a culture expressing the algae-RBD without a TEV protease site. “M” represents BLUelf™ Prestained Protein Ladder; “CS” represents 15 µL of concentrated supernatant. FIG. 8E shows a Western blot of 0.5 µL of concentrated supernatant (CS), and 10 ng of commercially available RBD made in HEK293 cells (+) with an anti-RBD antibody. FIG. 8F shows protein identification of the PtRBD by MALDI MS. The amino acid sequence of the PtRBD is shown and peptides identified are highlighted in yellow.

FIG. 9 shows the effect on electrophoretic mobility of treatment of purified PtRBD with the endoglycosidase PNGase F. A Western blot with an anti-RBD polyclonal antibody is shown.

FIG. 10 is an in vitro competitive inhibition assay of PtRBD and HEK293-RBD on immobilized angiotensin-converting enzyme-2 (ACE2) extracellular domain.

FIGS. 11A, 11B, and 11C is a lateral flow assay showing the ability of PtRBD to detect the presence of IgG antibodies in serum from patients previously infected with SARS-CoV-2 and from patients immunized with one or two doses of the Pfizer-BioNTech BNT162b2 vaccine. FIG. 11A shows results with an LFA prepared with PtRBD (“algae-RBD”) and FIG. 11B shows results with a commercially available RBD antigen (“DAGC174”) produced in mammalian cells. FIG. 11C shows representative results of LFA test strips comparing the performance of PtRBD (“algae-RBD”) and mammalian RBD (“DAGC174”) on the same patient samples side-by-side. Negative serum, confirmed negative by PCR; COVID-19 positive, confirmed positive by PCR; Double vaccination, serum from patients with two vaccine doses of Pfizer-BioNTech BNT162b2 vaccine and confirmed COVID-19 negative before vaccination.

FIGS. 12A, 12B, and 12C shows the purification of nucleocapsid protein (NC) expressed from the pSS40 plasmid expressed in P. tricornutum (PtNC). FIG. 12A shows a Coomassie blue stain; FIG. 12B shows a western blot using a polyclonal anti-nucleocapsid antibody as a primary antibody; and FIG. 12C shows a western blot using an anti-histidine antibody as a primary antibody. CRP; cross-reacting species.

FIGS. 13A, 13B, 13C, and 13D shows the results from the AKTA™ run at each purification step.

FIG. 14 shows the effect on electrophoretic mobility of treatment of purified PtNC with the endoglycosidase PNGase F. A Western blot with an anti-NC polyclonal antibody is shown.

FIG. 15 is a lateral flow assay showing the ability of PtNC to detect the presence of an anti-NC polyclonal antibody at ⅒, 1/100, and 1/1000 dilution. Also shown is a negative control without any anti-NC polyclonal antibody (right).

### SEQUENCE LISTING

This application contains a Sequence Listing in computer readable form created Aug. 30, 2022. The computer readable form is incorporated herein by reference.

TABLE 1

Sequence Listing Description

| SEQ ID NO: | Description |
|---|---|
| 1 | SARS-CoV-2 RBD amino acid sequence |
| 2 | pSS1 plasmid nucleotide sequence |
| 3 | pSS2 plasmid nucleotide sequence |
| 4 | pSS3 plasmid nucleotide sequence |
| 5 | pSS4 plasmid nucleotide sequence |
| 6 | pSS5 plasmid nucleotide sequence |
| 7 | pSS6 plasmid nucleotide sequence |
| 8 | pSS7 plasmid nucleotide sequence |
| 9 | pSS8 plasmid nucleotide sequence |
| 10 | Exemplary codon-optimized sequence for PtRBD |
| 11 | P. tricornutum HASP1 promoter version 1 |
| 12 | P. tricornutum HASP1 promoter version 2 |
| 13 | P. tricornutum HASP1 secretion signal peptide |
| 14 | SARS-CoV-2 native spike glycoprotein secretion signal peptide |
| 15 | P. tricornutum codon-optimized RBD coding sequence |
| 16 | P. tricornutum codon-optimized Spike protein coding sequence |
| 17 | P. tricornutum codon optimized Spike protein with 2 proline substitutions |
| 18 | Human codon optimized RBD |
| 19 | Human codon optimized Spike protein |
| 20 | SARS-CoV-2 RBD nucleotide sequence without a TEV protease cleavage site |
| 21 | pSS40 plasmid nucleotide sequence for Nucleocapsid expression |
| 22 | P. tricornutum codon-optimized Nucleocapsid coding sequence (excluding C-terminal 6xHis tag) |
| 23 | Nucleocapsid protein sequence expressed in P. tricornutum (excluding C-terminal 6xHis tag) |

### DETAILED DESCRIPTION

The marine pennate diatom Phaeodactylum tricornutum is a genetically tractable organism with a small, simple genome, a defined liquid growth media with requirement for light and oxygen, and scalable bioreactor culturing to volumes exceeding 10,000 L. Recent advancements in genetic tool development, such as efficient DNA delivery methods, replicating plasmids, antibiotic selection markers, and gene-editing systems, have enhanced the potential utility of P. tricornutum as an orthogonal production system. Despite such advancements, there are only a limited number of efficient inducible gene promoters available and there have been relatively few reports of the successful expression in P. tricornutum of exogenous glycoproteins, particularly those that are extensively glycosylated.

Here, we utilize P. tricornutum as an expression system for the overexpression and purification of the receptor-binding domain (RBD) of the spike and nucleocapsid proteins of SARS-CoV-2. We show that RBD produced in P. tricornutum (PtRBD) is N-linked glycosylated and can competitively inhibit binding of recombinant RBD produced in mammalian cell culture to the human ACE2 extracellular signalling domain. We also show that nucleocapsid protein produced in P. tricornutum (PtNC) is unglycosylated and is predominantly expressed as a N-terminally truncated protein that is nevertheless recognizable by control anti-nucleocapsid antibodies. Also demonstrated herein is the conjugation of PtRBD and PtNC, as well as their implementation in a functional lateral flow assay device and their potential to specifically bind IgG antibodies against the SARS-CoV-2 spike and nucleocapsid proteins present in serum from human patient samples. Overall, the results disclosed herein demonstrate that P. tricornutum represents a suitable expression system for low cost production of SARS-CoV-2 and other coronavirus glycoprotein or protein antigens.

It is further shown herein that recombinant gene expression under control of a P. tricornutum HASP1 (highly abundant secreted protein 1) promoter is responsive to inorganic phosphate levels in the culture media, with expression being repressed by higher phosphate levels. Interestingly, for recombinant proteins secretable by P. tricornutum, it is further shown herein that phosphate- and iron-limiting conditions may induce increased secretion of recombinant proteins under control of the HASP1 promoter. Indeed, the data presented herein suggest a number of strategies for phosphate-regulated expression based on titrating media phosphate that would be particularly applicable, for example, for the expression of toxic proteins and/or for timing expression for particular growth stages.

In a first aspect, described herein is a recombinant glycoprotein or protein expressed in P. tricornutum, comprising a polypeptide antigen (e.g., viral, bacterial, or fungal antigen), the recombinant glycoprotein or protein having a glycosylation or other post-translational modification pattern similar to that produced in mammalian (e.g., human) expression systems. In some implementations, recombinant glycoproteins or proteins described herein may comprise a polypeptide antigen that is extensively glycosylated when expressed in their native host cells, such as a coronavirus polypeptide antigen (Grant et al., 2020; Walls et al., 2016).

In some implementations, recombinant glycoproteins or proteins described herein may comprise a coronavirus polypeptide antigen having a glycosylation pattern (e.g., N-linked glycosylation pattern and/or O-linked glycosylation pattern and/or phosphorylation pattern) produced by, or characteristic of, post-translational modification by P. tricornutum. In some implementations, recombinant glycoproteins or proteins described herein may comprise a coronavirus polypeptide antigen, such as a betacoronavirus polypeptide antigen. In some implementations, the betacoronavirus polypeptide antigen may be from a SARS-CoV-2, SARS-CoV, or MERS-CoV polypeptide antigen.

In another aspect, described herein is a recombinant protein expressed in P. tricornutum, comprising a polypeptide antigen (e.g., viral, bacterial, or fungal antigen), the recombinant protein having similar post-translational modifications to that produced in mammalian (e.g., human) expression systems. In some implementations, recombinant proteins described herein may comprise a polypeptide antigen that is not extensively glycosylated when expressed in their native host cells, such as a coronavirus Nucleocapsid protein.

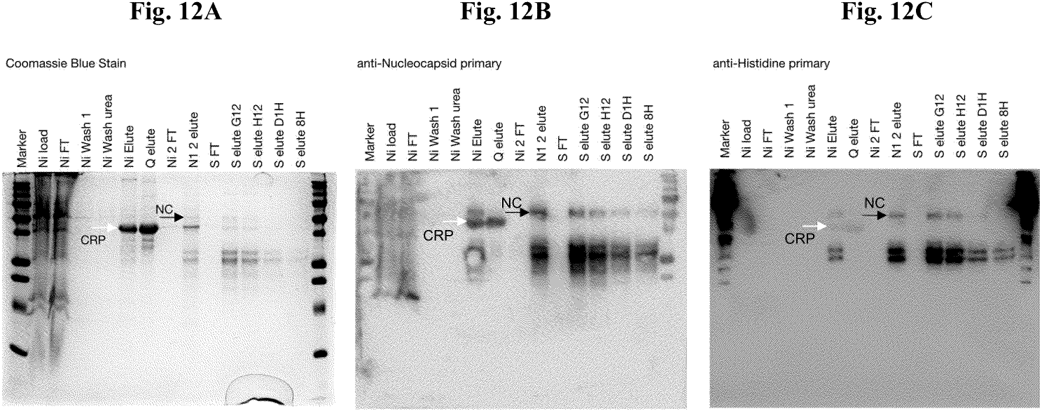

In some implementations, recombinant glycoproteins described herein may be from or comprise a major structural protein encoded by a pathogenic viral genome. In some implementations, recombinant glycoproteins described herein may be from or comprise a surface glycoprotein (e.g., spike (S) protein, a nucleocapsid (N) protein, a membrane protein, or an envelope protein). In some implementations, polypeptide antigens described herein may be or comprise a fragment of a coronavirus spike protein (e.g., a fragment comprising Sl subunit, S2 subunit, or receptor binding domain). In some implementations, polypeptide antigens described herein may be or comprise a fragment of a coronavirus spike protein’s RBD, wherein the RBD retains biological activity. In some implementations, the RBD biological activity may comprise the ability of the RBD (comprised in a recombinant glycoprotein or protein described herein) to bind to its corresponding receptor on its target host cell (e.g., human angiotensin-converting-enzyme 2 [ACE2] receptor, in the case of SARS-CoV-2). In some implementations, polypeptide antigens described herein may be or comprise a fragment of a coronavirus nucleocapsid protein (e.g., an N-terminally truncated nucleocapsid protein, such as an N-terminally truncated nucleocapsid protein lacking contiguous residues 19-110, 19-111, 19-112, 19-113, 19-114, 19-115, 19-116, 19-117, 19-118, 19-119, 19-120, 19-121, 19-122, 19-123, 19-124, 19-125, 19-126, 19-127, 19-128, 19-129, 19-130, 19-131, 19-132, 19-133, 19-134, 19-135, 19-136, 19-137, 19-138, 19-139, 19-140, 19-141, 19-142, 19-143, 19-144, 19-145, 19-146, 19-147, 19-148, 19-149, 19-150, 19-151, 19-152, 19-153, 19-154, 19-155, 19-156, 19-157, 19-158, 19-159, 19-160, 19-161, 19-162, 19-163, 19-164, 19-165, 19-166, 19-167, 19-168, 19-169, 19-170, 19-171, 19-172, 19-173, 19-174, 19-175, 19-176, 19-177, 19-178, 19-179, 19-180, 19-181, 19-182, 19-183, 19-184, 19-185, 19-186, 19-187, 19-188, 19-189, 19-190, 19-191, 19-192, 19-193, 19-194, 19-195, 19-196, 19-197, 19-198, 19-199, 19-200, 19-201, 19-202, 19-203, 19-204, 19-205, 19-206, 19-207, 19-208, 19-209, 19-210, 19-211, 19-212, 19-213, 19-214, 19-215, 19-216, 19-217, 19-218, 19-219, 19-220, 19-221, 19-222, 19-223, 19-224, 19-225, 19-226, 19-227, 19-228, 19-229, 19-230, 19-231, or 19-232 of SEQ ID NO: 23). In this regard, residues 1-18 of SEQ ID NO: 23 correspond to a leader sequence for expression in [symbol] that is not part of the native SARS-CoV-2 nucleocapsid protein. Furthermore, the aforementioned contiguous residues absent from the truncated PtNC protein is confirmed by the increased electrophoretic mobility by SDS-PAGE (FIGS. 12A-12C and 14), as well as by analysis by mass spectrometry.

In some implementations, the RBD or nucleocapsid biological activity may comprise the ability of the RBD or nucleocapsid (comprised in a recombinant protein described herein) to competitively inhibit the binding of a native RBD or nucleocapsid protein produced in mammalian (e.g., human) cells to its target host cell (e.g., human ACE2 receptor, in the case of SARS-CoV-2). In some implementations, within a diagnostic and/or immunogen context, the RBD or nucleocapsid biological activity may comprise the ability of the ability of the RBD or nucleocapsid (comprised in a recombinant protein described herein) to bind to (or cross-react with) antibodies (e.g., neutralizing antibodies) raised against the spike or nucleocapsid protein or an RBD or nucleocapsid-comprising fragment thereof.

In some implementations, recombinant glycoproteins or proteins described herein may comprise an amino acid sequence at least 70%, 75%, 80%, 85%, 90%, 95%, or 99% identical to SEQ ID NO: 1. In some implementations, recombinant glycoproteins or proteins described herein may comprise an amino acid sequence at least 70%, 75%, 80%, 85%, 90%, 95%, or 99% identical to SEQ ID NO: 23, or to residues 19-443 of SEQ ID NO: 23, optionally further comprising an N-terminal sequence comprising residues 1-18 of SEQ ID NO: 23.

In some implementations, recombinant glycoproteins described herein may possess an N-linked glycosylation pattern comprising N-linked glycans at positions N13 and N25 (using the residue numbering of SEQ ID NO: 1). In some implementations, recombinant glycoproteins may comprise a mixture of complex glycans, such as core fucosylation.

In some implementations, recombinant glycoproteins or proteins described herein may have an overall length of no more than 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1000 residues.

In some implementations, recombinant glycoproteins or proteins described herein may lack (or be engineered to lack) a functional endoplasmic reticulum retention signal, thereby enabling the formation of more complex N-linked glycosylation in the Golgi apparatus of P. tricornutum host cells.

In some implementations, recombinant glycoproteins or proteins described herein may include (or be engineered to include) a cleavable purification tag, such as but not limited to a glutathione-S-transferase tag, a His tag (e.g., 6His or 10His), or an Fc tag. In some implementations, the cleavable purification tag is not fused to recombinant glycoproteins or proteins described herein via a Tobacco Etch Virus (TEV) protease cleavage site. In some implementations, the recombinant glycoproteins described herein lack (or are engineered to lack) a sequence cleavable by a TEV protease. As used herein, the expression “TEV protease cleavage site” or “sequence cleavable by a TEV protease” refers not only to the amino acid recognition sequence that results in optimal or near optimal TEV protease cleavage, but also encompasses variants of the optimal TEV protease recognition sequence that may result in detectable cleavage by the protease (Kapust et al., 2002; Kostallas et al., 2011).

In some implementations, recombinant glycoproteins or proteins described herein may be comprised in (or used for the manufacture of) a kit or device for detecting the presence and/or concentration of antibodies that bind to the recombinant protein. In some implementations, recombinant glycoproteins or proteins described herein may be comprised in (or used for the manufacture of) an immunogenic composition (e.g., a vaccine) for use in triggering the production of antibodies against said recombinant protein in a subject.

In a further aspect, described herein is a P. tricornutum host cell that produces, or is engineered to produce, a recombinant glycoprotein or protein as described herein. In some implementations, the host cell comprises an exogenous expression cassette encoding the recombinant glycoprotein or proteins operably linked to a promoter.

In a further aspect, described herein is a diagnostic device comprising a recombinant protein described herein for use in detecting the presence and/or concentration of antibodies that bind to said recombinant protein. In some implementations, the diagnostic device may be or comprise a lateral flow assay (LFA) test or ELISA. In a further aspect, described herein is a method for detecting antibodies specific to a polypeptide antigen (e.g., a coronavirus polypeptide antigen) in a biological sample, the method comprising: (a) contacting the biological sample with the recombinant glycoprotein or protein as described herein; and (b) detecting a complex formed between antibodies specific to the polypeptide antigen and the recombinant glycoprotein or protein.

In a further aspect, described herein is an immunogenic composition (e.g., vaccine) comprising the recombinant glycoprotein or protein as described herein, and a suitable adjuvant. In a further aspect, described herein is an antigen-antibody complex comprising a recombinant glycoprotein or protein as described herein and an antibody bound thereto (e.g., anti-coronavirus antibody that cross-reacts therewith), wherein the antibody is from a biological sample from a subject (e.g., a blood sample from a human subject). In a further aspect, described herein is a method for triggering the production of antibodies against a polypeptide antigen (e.g., a coronavirus polypeptide antigen), the method comprising administering to a subject an immunogenic composition as described herein.

In a further aspect, described herein a polynucleotide encoding a recombinant protein as described herein. In some implementations, the polynucleotide may be codon-optimized for increased expression in P. tricornutum host cells, relative to a polynucleotide encoding a corresponding protein using unoptimized or native codons. In some implementations, the polynucleotide encoding a codon-optimized RBD or spike protein of SARS-CoV-2 is as set forth in any one of SEQ ID NOs: 2-10 or 15-19 (e.g., the RBD-encoding sequence of SEQ ID NO: 10). In some implementations, the polynucleotide encoding a codon-optimized nucleocapsid protein of SARS-CoV-2 is as set forth in SEQ ID NO: 22 or in nucleotides 115-1329 of SEQ ID NO: 22. In some implementations, the polynucleotide further encodes a cleavable purification tag other than a TEV protease cleavage site. In some implementations, polynucleotides described herein do not encode (or are engineered to avoid encoding) an amino acid sequence that is cleavable by TEV protease.

In a further aspect, described herein is an expression cassette or expression vector (e.g., plasmid) comprising a polynucleotide as defined herein operably linked to a promoter. In some implementations, the promoter is heterologous with respect to the polynucleotide and/or the host cell. In some implementations, described herein is a P. tricornutum chromosome comprising an expression cassette as described herein.

In a further aspect, described herein is a method for producing a recombinant protein, the method comprising providing P. tricornutum or other suitable host cells (e.g., diatom host cells or cells of the same clade of P. tricornutum) comprising a polynucleotide encoding the recombinant protein in an expression cassette under control of an HASP1 (highly abundant secreted protein 1) promoter; and culturing the host cells in a production medium for a sufficient period of time to induce expression of the recombinant protein. In some implementations, method may be divided into a growth phase favoring the accumulation of cell mass, and a production phase favoring recombinant protein expression. In some implementations, method may comprise a combined growth and production phase favoring both the accumulation of cell mass and recombinant protein expression. In some implementations, method may comprise toggling between growth and production phases.

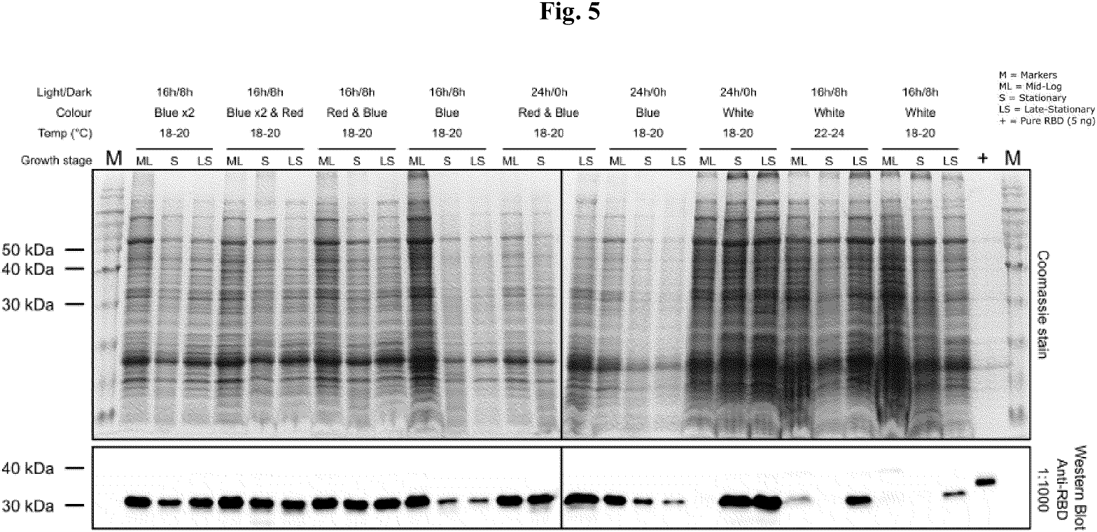

As used herein, the expression “HASP1 promoter” refers to the genetic elements within the endogenous promoter region of the diatom HASP1 (highly abundant secreted protein 1) gene, preferably from P. tricornutum (e.g., as characterized in Erdene-Ochir et al., 2019), which is sufficient for gene expression in response to changes in phosphate levels. In some implementations, the HASP1 promoter may comprise one or more putative phosphate-regulatory motifs (P1BS-like, FIG. 5A). The HASP1 promoter sequences employed in this study are set forth within the plasmid sequences of SEQ ID NOs: 2-9 or 15-19.

As used herein, the expression “suitable host cells” or “suitable microorganisms”, in the context of using phosphate/iron levels to control expression/secretion of a recombinant protein expressed under control of an HASP1 promoter, refers to cells or microorganisms that have the cellular machinery to make use of the regulatory elements present in the HASP1 promoter. In some implementations, such suitable cells or microorganisms may potentially be inferable from genomic sequence analyses to determine the level of conservation of the regulatory elements in the cell’s or native microorganism’s genome.

In some implementations, the growth phase may be performed by culturing the host cells in a growth medium having a nutrient composition optimized for cell growth. In some implementations, the production phase may be performed by culturing the host cells in a production medium having a nutrient composition optimized for recombinant protein expression and/or recombinant protein secretion.

In some implementations, the production medium may have or be maintained at an inorganic phosphate concentration sufficiently low such that the recombinant protein is expressed at a level higher than when the host cells are cultured under corresponding conditions in a phosphate-replete medium. As used herein, the expression “phosphate-replete medium” refers to a medium having a concentration of inorganic phosphate that is non-limiting to the host cells (e.g., in the context of cell growth, which can be assessed as shown in FIG. 5C). In some implementations, the production medium may be a phosphate-reduced production medium having an inorganic phosphate concentration sufficiently low such that the recombinant protein is expressed at a level at least 1.2, 1.3, 1.4, 1.5, 1.6, 1.7, 1.8, 1.9, or 2-fold higher than when the host cells are cultured under corresponding conditions in a phosphate-replete medium. In some implementations, the production medium may be a phosphate-reduced production medium having an inorganic phosphate concentration of less than or equal to 50%, 45%, 40%, 35%, 30%, 25%, 20%, 15%, 10%, or 5% of that present in a phosphate-replete growth medium that was used to culture the host cells provided. In some implementations, the production medium may be an inorganic phosphate concentration of less than or equal to 30, 29, 28, 27, 26, 25, 24, 23, 22, 21, 20, 19, 18, 17, 16, 15, 14, 13, 12, 11, 10, 9, 8, 7, 6, 5, 4, 3, 2.5, 2, 1.5, or 1 µM. In some implementations, the production medium may be an inorganic phosphate concentration of less than or equal to 2.8, 2.7, 2.6, 2.5, 2.4, 2.3, 2.2, 2.1, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1 ppm. In some implementations, the production medium may be any combinations of the above-mentioned implementations.

In some implementations, the host cells, prior to a production phase, may be cultured in a phosphate-replete growth medium having an inorganic phosphate concentration sufficiently high to repress expression of the recombinant protein as compared to when the host cells are cultured in a phosphate-reduced production medium (a medium in which inorganic phosphate concentration is cell growth-limiting).

In some implementations, the growth medium and/or production medium may be a phosphate-reduced and iron-reduced medium. Interestingly, growth curves in FIG. 5C revealed that while P. tricornutum grew at slower rates when either phosphate or iron was limited, cells cultured in media in which both phosphate and iron were limited grew at the same rate as in full L1 media. In some implementations, a phosphate-reduced and iron-reduced production medium may be employed when the recombinant protein is to be secreted from the host cells. While limiting phosphate stimulated robust recombinant eGFP expression (Example 3), only in media with a similar reduction in iron was secretion observed (FIG. 5D).

In some implementations, the iron-reduced medium and/or a phosphate-reduced and iron-reduced medium described herein may have an inorganic phosphate concentration as defined herein and: an iron concentration sufficiently low such that the recombinant protein is secreted at a level at least 1.2, 1.3, 1.4, 1.5, 1.6, 1.7, 1.8, 1.9, or 2-fold higher than when the host cells are cultured under corresponding conditions in an iron-replete medium; an iron concentration of less than or equal to 50%, 45%, 40%, 35%, 30%, 25%, 20%, 15%, 10%, or 5% of that present in an iron-replete growth medium that was used to culture the host cells provided herein; an iron concentration of less than or equal to: 10, 9, 8, 7, 6, 5, 4, 3, 2.5, 2, 1.5, 1, 0.9, 0.8, 0.7, 0.6, 0.5, 0.4, 0.3, 0.25, 0.2, 0.15, or 0.1 µM; or any combination thereof. As used herein, the expression “iron-replete medium” refers to a medium having a concentration of iron that is non-limiting to the host cells (e.g., in the context of cell growth, which can be assessed as shown in FIG. 5C). In some implementations, the host cells, prior to a production phase, may be cultured in an iron-replete growth medium having an iron concentration sufficiently high to repress secretion of the recombinant protein as compared to when the host cells are cultured in an iron-reduced production medium. In some implementations, the host cells, prior to a production phase, may be cultured in a phosphate-reduced and iron-reduced medium as defined herein as a growth medium.

In some implementations, the host cells may be cultured in the production medium for at least 1, 1.5, 2, 2.5, 3, 3.5, 4, 4.5, or 5 days. In some implementations, the host cells may be engineered to comprise an expression cassette as described herein as part of their genome. In some implementations, the recombinant protein may be heterologous with respect to the host cells and/or with respect to the HASP1 promoter. In some implementations, the recombinant protein may be a recombinant glycoprotein as described herein.

In a further aspect, described herein is a method for regulating the production of a recombinant protein, the method comprising providing a culture of suitable host cells (e.g., P. tricornutum or other suitable microorganisms such as diatoms) comprising a polynucleotide encoding the recombinant protein in an expression cassette under control of an HASP1 (highly abundant secreted protein 1) promoter; and controlling the inorganic phosphate levels in the culture to regulate the expression of the recombinant protein, wherein the inorganic phosphate levels are maintained above a repression threshold level for a sufficient period of time when expression of the recombinant protein is to be repressed, and wherein the inorganic phosphate levels are maintained below an induction threshold level for a sufficient period of time when expression of the recombinant protein is to be induced. In some implementations, the induction threshold level is an inorganic phosphate concentration sufficiently low such that the recombinant protein is expressed at a level at least 1.2, 1.3, 1.4, 1.5, 1.6, 1.7, 1.8, 1.9, or 2-fold higher than when the host cells are cultured under corresponding conditions in a phosphate-replete medium or above the repression threshold level. In some implementations, the repression threshold level may be above 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, or 400 µM. In some implementations, the induction threshold level may be below 30, 29, 28, 27, 26, 25, 24, 23, 22, 21, 20, 19, 18, 17, 16, 15, 14, 13, 12, 11, 10, 9, 8, 7, 6, 5, 4, 3, 2.5, 2, 1.5, or 1 µM. In some implementation, the method is for regulating the production and secretion of the recombinant protein, wherein the method further comprises controlling the iron levels in the culture to regulate the secretion of the recombinant protein, wherein the inorganic phosphate levels are maintained below the induction threshold level and the iron levels are maintained above a secretion threshold level for a sufficient period of time when secretion of the recombinant protein is to be repressed, and wherein the iron levels are maintained below a secretion threshold level for a sufficient period of time when secretion of the recombinant protein is to be induced. In some implementation, the secretion induction threshold level is: an iron concentration sufficiently low such that the recombinant protein is secreted at a level at least 1.2, 1.3, 1.4, 1.5, 1.6, 1.7, 1.8, 1.9, or 2-fold higher than when the host cells are cultured under corresponding conditions in an iron-replete medium or at the secretion repression threshold level; and/or an iron concentration of less than or equal to: 10, 9, 8, 7, 6, 5, 4, 3, 2.5, 2, 1.5, 1, 0.9, 0.8, 0.7, 0.6, 0.5, 0.4, 0.3, 0.25, 0.2, 0.15, or 0.1 µM.

In a further aspect, described herein is a method for increasing secretion and/or expression of a recombinant protein being expressed in an algae microorganism (e.g., P. tricornutum), said method comprising: providing a host cell algae microorganism comprising an expression cassette or vector comprising a polynucleotide encoding the recombinant protein and does not encode for a Tobacco Etch Virus (TEV) protease cleavage site; and culturing the host cells in a production medium for a sufficient period of time to induce expression and/or secretion of the recombinant protein.

In a further aspect, described herein is an expression cassette or vector (e.g., plasmid) for use in increasing the expression and/or secretion of a recombinant protein in a host cell algae microorganism (e.g., P. tricornutum), said expression cassette or vector comprising a polynucleotide encoding the recombinant protein and which does not encode a Tobacco Etch Virus (TEV) protease cleavage site

In a further aspect, described herein is a method for targeting a recombinant glycoprotein or protein expressed in Phaeodactylum tricornutum for degradation and/or for intracellular retention. In some embodiments, the method generally comprises engineering a TEV protease cleavage site into the recombinant glycoprotein or protein, and culturing the P. tricornutum such that the recombinant glycoprotein or protein is cleaved by an endogenous protease of the P. tricornutum, thereby targeting the recombinant glycoprotein or protein for degradation and/or intracellular retention. In some embodiments, “intracellular retention” refers to reduced extracellular expression of the recombinant glycoprotein or protein comprising the TEV protease cleavage site as compared the level of expression of a corresponding recombinant glycoprotein or protein lacking the TEV protease cleavage site.

In some aspects, there is described a a method for producing or modifying a SARS-CoV-2 test (e.g., detection presence of infection or immunity), the method comprising adding or integrating into said test quantifying or detecting the presence or level of the recombinant glycoprotein or protein produced by the method described herein.

### EXAMPLES

### Example 1: Materials and Methods

### Microbial Strains and Growth Conditions

Saccharomyces cerevisiae VL6-48 (ATCC MYA-3666: MATα his3-Δ200 trp1-Δ1 ura3-52 lys2 ade2-1 met14 cir⁰ was grown in YPD medium or complete minimal medium lacking histidine (Teknova™) supplemented with 60 mg/L adenine sulfate. Complete minimal media used for spheroplast transformation contained 1 M sorbitol. E. coli (Epi300™, Epicentre) was grown in Luria™ Broth (LB) supplemented with appropriate antibiotics (chloramphenicol (25 mg/L) or ampicillin (50 mg/L) or gentamicin (20 mg/L)). P. tricornutum (Culture Collection of Algae and Protozoa CCAP 1055/1) was grown in L1 medium without silica, with or without histidine (200 mg/L), supplemented with appropriate antibiotics (Zeocin™ (50 mg/L) or nourseothricin (150 mg/L)), at 18° C. under cool white fluorescent lights (75 µE m⁻²s⁻¹) and a photoperiod of 16 h light:8 h dark. L1 media supplemented with nourseothricin contained half the normal amount of aquil salts. Unless otherwise stated, the L1 media used herein is a modified L1 media containing 362 micromolar phosphate, corresponding to 10-fold higher phosphate than present in, for example, standard algae media (e.g., standard L1 medium or standard F/2 medium). This media condition was found in our previous work to yield much higher cell densities. A comparison of media compositions is shown in the Table below.

| | | | |
|---|---|---|---|
| Component | Modified L1 media | Standard L1 media | f/2 media |
| NaNO₃ | 75 mg L⁻¹ | 7.5 mg L⁻¹ | 7.5 mg L⁻¹ |
| NaH₂PO₄·H₂O | 50 mg L⁻¹ | 5 mg L⁻¹ | 5 mg L⁻¹ |
| FeCl₃·6H₂O | 3.15 mg L⁻¹ | 3.15 mg L⁻¹ | 3.15 mg L⁻¹ |
| Na₂EDTA·2H₂O | 4.36 mg L⁻¹ | 4.36 mg L⁻¹ | 4.36 mg L⁻¹ |
| CuSO₄·5H₂O | 2.45 µg L⁻¹ | 2.5 µg L⁻¹ | 9.8 µg L⁻¹ |
| Na₂MoO₄·2H₂O | 18.9 µg L⁻¹ | 19.9 µg L⁻¹ | 6.3 µg L⁻¹ |
| ZnSO₄·7H₂O | 22 µg L⁻¹ | 23 µg L⁻¹ | 22 µg L⁻¹ |
| CoCl₂·6H₂O | 10 µg L⁻¹ | 11.9 µg L⁻¹ | 10 µg L⁻¹ |
| MnCl₂·4H₂O | 180 µg L⁻¹ | 178.1 µg L⁻¹ | 180 µg L⁻¹ |
| H₂SeO₃ | 1.3 µg L⁻¹ | 1.29 µg L⁻¹ | n/a |
| NlSO₄·6H₂O | 2.7 µg L⁻¹ | 2.63 µg L⁻¹ | n/a |
| Na₃VO₄ | 1.84 µg L⁻¹ | 1.84 µg L⁻¹ | n/a |
| K₂CrO₄ | 1.94 µg L⁻¹ | 1.94 µg L⁻¹ | n/a |
| Na₂SiO₃·9H₂O | n/a | 30 mg L⁻¹ | n/a |
| Na₂CO₃ | n/a | n/a | 30 mg L⁻¹ |

Standard L1 and f/2 media compositions from Guillard, et al., 1962. Guillard, et al., 1975.

### Plasmid Design and Construction

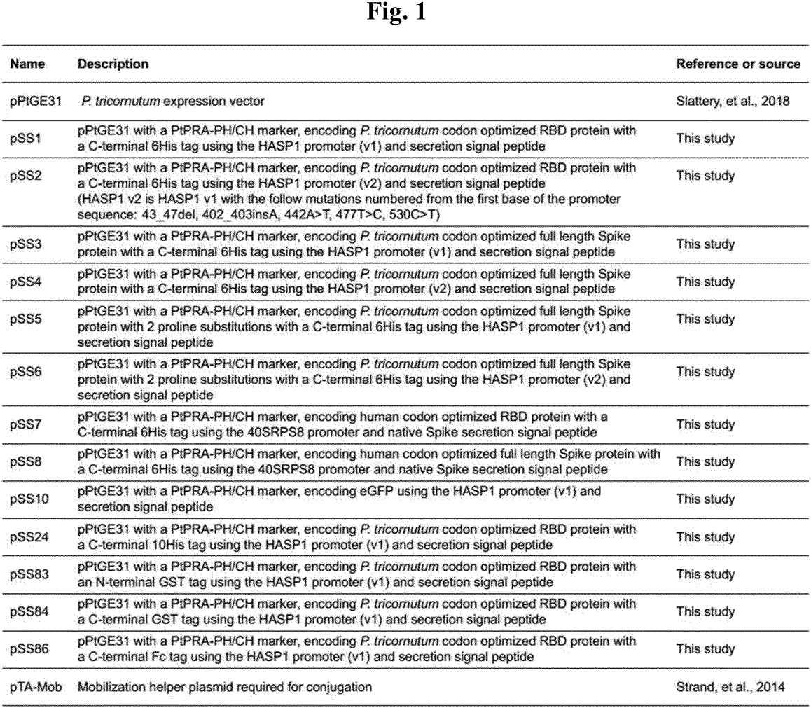

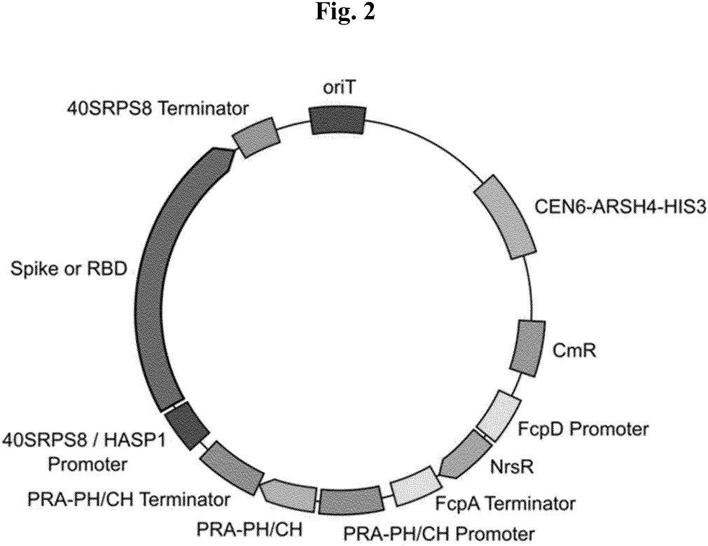

All plasmids were constructed using a modified yeast assembly protocol (Gibson et al., 2006; Noskov et al., 2012). We cloned versions of the SARS-CoV-2 spike protein gene into the E. coli / P. tricornutum shuttle plasmid pPtGE31 (Slattery et al., 2018). We obtained SARS-CoV-2 expression plasmids from the Krammer lab in New York city. These served as templates for PCR amplification of the human codon-optimized spike and receptor-binding domain coding regions for cloning into P. tricornutum expression plasmids. We also ordered synthetic constructs corresponding to the full-length spike protein gene and RBD from IDT-DNA that were codon-optimized for P. tricornutum. The nine initial constructs are listed in FIG. 1 and a representative schematic can be seen in FIG. 2. The polynucleotide sequences of the plasmids pSS1, pSS2, pSS3, pSS4, pSS5, pSS6, pSS7, and pSS8 are provided in SEQ ID NOs: 2-9 or 15-19. The constructs differed in the following ways: codon-optimization for human or P. tricornutum, full-length or RBD of the spike protein, version 1 or version 2 of the P. tricornutum HASP1 promoter (originating from two homologous P. tricornutum chromosomes). We also cloned a version of the full-length construct with two proline stabilizing mutations and a mutation of the furin cleavage site (RRAR) to an alanine, in addition to a stabilizing trimerization motif. The constructs were made with a promoter from the P. tricornutum 40SRPS8 (40S ribosomal protein S8) gene or from the HASP1 gene (highly abundant secreted protein 1). All constructs contained the 40SRPS8 terminator downstream of the spike or RBD coding sequence. All constructs also included a histidine marker (PRA-PH/CH) expression cassette from the pPtPRAPHCH plasmid (Slattery et al., 2020) for selection and maintenance in a P. tricornutum histidine auxotroph strain. Plasmids encoding the P. tricornutum codon-optimized versions had the HASP1 promoter and the HASP1 secretory signal peptide, while plasmids encoding the human codon-optimized versions used the 40SRPS8 promoter and the native spike protein secretory signal. The plasmid constructs were assembled in yeast by co-transforming linear DNA fragments of the pPtGE31 plasmid backbone, the PRA-PH/CH expression cassette, the HASP1 or 40SRPS8 promoter, and the spike or RBD protein gene. Resultant yeast colonies were pooled, DNA extracted, and transformed into E. coli. Single E. coli colonies were grown and plasmid DNA isolated using standard plasmid mini-prep kits. Correct assembly was confirmed by restriction enzyme digests, by whole-plasmid sequencing using a Minion sequencer from Nanopore, and by sequencing of the spike and RBD protein expression cassettes at the London Regional Genomics Centre.

### Plasmid Validation

Plasmid DNA was extracted using the NEB miniprep kit (T1010L). 400 ng of DNA was used as input for the rapid barcoding kit library prep (SQK-RBK004). Plasmids were then sequenced using R9.4.1 Flongle flow cells (FLO-FLG001) or R9.4.1 minION™ flow cells until approximately 200X coverage was obtained for each barcode based on an expected plasmid size of 20 kilobases. Basecalling was performed using Guppy™ v4.2.2 in high-accuracy mode (Oxford Nanopore Technologies). Reads were filtered by retaining only those near the expected plasmid length. Reads were then assembled using miniasm™ (Li et al., 2016). The assembly was then polished using minipolish™ (Wick et al., 2019) and medaka™ (Oxford Nanopore Technologies). Polished assemblies were then compared to the expected sequence to determine if any mutations were present.

### RNAseq Analysis

Total RNA was extracted from 15 mL cultures with an OD₆₇₀ of 0.6-0.7 by first crushing the algal cells in liquid nitrogen as follows. Cultures were centrifuged at 3000 g for 15 mins at 4° C. The pellet was resuspended in ~100-500 µL TE pH 8.0 and added dropwise to a mortar (pre-cooled at -80° C.) filled with liquid nitrogen. The frozen droplets were ground into a fine powder with a mortar and pestle, being careful to keep the cells from thawing by adding more liquid nitrogen when necessary. The frozen ground powder was transferred to a new clean 1.5 mL microfuge tube and stored at -80° C. RNA was extracted from 50-100 mg of frozen ground powder with the Monarch™ Total RNA Miniprep Kit (T2010S) following the plant protocol. The RNA was stored in TE pH 8.0 at -80° C. until use. Quantity and purity were measured by spectrophotometer, and RNA integrity was evaluated using a 1% pre-stained agarose gel run at 100 V for 30 minutes. RNA integrity was further evaluated using an Agilent Bioanalyzer. rRNA was depleted using the Vazyme Ribo-off™ plant rRNA depletion kit (N409). Sequencing libraries were then prepared and sequenced by the London Regional Genomics Center (lrgc.ca) using an Illumina NextSeq™ high output single output 75 run. Reads were trimmed to 75 base pairs and aligned against the ASM15095v2 reference assembly and expected plasmid sequence using hisat2 (Kim et al., 2019). Coverage was determined using htseq (Anders et al., 2015).

### Diagnostic PCR Assays

For direct PCR assays, dilutions of RBD expression cultures were plated onto modified L1 with nourseothricin (100 mg/L) and screened for the presence of the RBD gene using a Thermo Scientific Phire™ Plant Direct PCR Master Mix according to manufacturers instructions. PCR screens were performed using a forward primer located in the HASP1 promoter (DE5241) for P. tricornutum transformed with pSS1 and pSS2, or in the 40SRPS8 promoter (DE4130) for P. tricornutum transformed with pSS7. Reverse primers were positioned inside the RBD domain coding regions (DE5323, DE5326).

### Transfer of DNA to P. Tricornutum Via Conjugation From E. Coli

Conjugations were performed as previously described (Karas et al., 2015; Slattery et al., 2018). Briefly, liquid cultures (250 mL) of P. tricornutum, adjusted to a density of 1.0×10⁸ cells/mL using counts from a hemocytometer, were plated on ½×L1 1% agar plates with or without histidine (200 mg/L), and grown for four days. L1 media (1.5 mL) was added to the plate and cells were scraped and the concentration was adjusted to 5.0×10⁸ cells/mL. E. coli cultures (50 mL) were grown at 37° C. to A₆₀₀ of 0.8-1.0, centrifuged for 10 mins at 3,000 × g and resuspended in 500 mL of SOC media. Conjugation was initiated by mixing 200 µL of P. tricornutum and 200 µL of E. coli cells. The cell mixture was plated on ½×L1 5% LB 1% agar plates, incubated for 90 mins at 30° C. in the dark, and then moved to 18° C. in the light and grown for 2 days. After 2 days, L1 media (1.5 mL) was added to the plates, the cells scraped, and 300 µL (20%) plated on ½×L1 1% agar plates supplemented with Zeocin 50 mg/L or nourseothricin 100 mg/L. Colonies appeared after 7-14 days incubation at 18° C. with light.

### Measuring Growth and eGFP Production of P. Tricornutum Cultures

P. tricornutum cultures were adjusted to an OD₆₇₀ of 0.05 in L1 media made without phosphate, nitrate, or iron. Cultures were then washed by centrifugation for 10 mins at 3,000 × g followed by resuspension in fresh L1 media without phosphate, nitrate, or iron. Phosphate, nitrate, and iron stock solutions were then used to adjust cultures to the follow conditions: full L1 or L1 with 5% phosphate, 5% nitrate, 5% iron, 5% phosphate and 5% iron, 5% phosphate and 5% nitrate, or 5% nitrate and 5% iron. Cultures were grown at 18° C. under cool white fluorescent lights (75 µE m⁻²s⁻¹) and a photoperiod of 16h light:8h dark for 28 days, and absorbance at 670 nm (A₆₇₀) was measured every 48 h using an Ultrospec™ 2100 pro UV/vis spectrophotometer. Samples for fluorescence readings and Western blots were taken every 4 days by centrifuging 700 µL of culture at 16,000 × g for 15 minutes. Three 200 µL aliquots of the supernatant were pipetted into a clear bottom 96-well plate for fluorescence readings. Another 44 µL of supernatant was mixed with 22 µL of 3X SDS sample loading buffer (187.5 mM Tris-HCl (pH 6.8), 6% (w/v) SDS, 30% [v/v] glycerol, 150 mM DTT, 0.03% (w/v) bromophenol blue, 2% [v/v] b-mercaptoethanol) and boiled at 95° C. for 10 mins, after which 15 µL of boiled sample was analyzed by Western blot. The pellet was resuspended in 50 µL of 3X SDS sample loading buffer and boiled at 95° C. for 10 mins, after which 10 µL of boiled sample was analyzed by Western blot. Fluorescence readings were taken in a Biotek Synergy™ H1 plate reader at an excitation wavelength of 475 nm and emission wavelength of 515 nm. Fluorescence values obtained were subtracted from wildtype autofluorescence in the supernatant. Fluorescence values were converted to eGFP in µg/mL using a standard curve generated using commercially available purified eGFP.

### Bioreactor Conditions for Growth of P. Tricornutum

A 5-L bioreactor system was used for the growth of P. tricornutum. Temperature was controlled in the bioreactor at 18° C. Mixing was achieved with a single marine type blade impeller at 100 rpm. A constant gas flow of 0.75 VVM was sparged into the reactor with a mix of 0.5% carbon dioxide and 99.5% air. The pH of the culture was controlled at 8.1 using a cascade with carbon dioxide from 0.5-5% v/v mix. Light was provided by continuous (24 hours/day) full spectrum LED grow lights with 5 bulbs at a light intensity of approximately 50 mol m⁻² s⁻¹. Samples were collected daily for optical density, cell count and compositional analysis. A 10% inoculum was used to attain a minimum cell density of 2 million cells/mL. The inoculum was cultured in an incubator (Innova S44i™, Eppendorf, Hamburg, Germany) with a photosynthetic light bank containing LED lighting. Lighting for the inoculum was at an intensity of approximately 65 mol m⁻² s⁻¹ with a cycling of 16 hours on and 8 hours off. Temperature was controlled at 18° C. with orbital agitation at 100 rpm. pH was not controlled, and no gas was sparged into the inoculum.

### Compositional Analysis of Media

The concentration of dissolved phosphorus and iron was measured by Inductively Coupled Plasma-Optical Emission Spectrometry (ICP-OES). An Agilent 5110 (Agilent, USA) spectrometer ICP-OES equipped with a Seaspray™ concentric glass nebulizer (Agilent, USA) and an SPS 4 auto sampler was used. Argon (purity higher than 99.995%) supplied by Linde Canada was used to sustain plasma as carrier gas. The operating conditions employed for ICP-OES determination were 1200 W RF power, 12 L/min plasma flow, 1.0 L/min auxiliary flow, 0.7 L/min nebulizer flow, with radial view used for determination. The most sensitive lines free of spectral interference were used to determine emission intensities. The calibration standards were prepared by diluting a phosphorus and iron standards (Agilent, USA) in synthetic seawater and 1% (v/v) nitric acid. The calibration curves for both elements were in the range of 0.05 to 5 ppm.

### Protein Extraction and Purification of SARS-CoV-2 Spike RBD

P. tricornutum cultures (5 L) were harvested during stationary growth phase and pelleted at 3,000 × g for 10 mins at 4° C. Cell pellets were resuspended in lysis buffer (50 mM Tris-HCl pH 7.4, 0.5 M NaCl, 10 mM imidazole, 0.1% Tween™-20, 1 mM DTT, 1X Protease inhibitor cocktail (Sigma)) and homogenized on an Emulsiflex™ C3 Homogenizer with 5 passes at 20,000 psi to lyse. Sonicated lysates were centrifuged at 20,000 × g for 30 mins at 4° C. to pellet cell debris, and supernatants were collected in a new tube and stored on ice before purification using a 20 mL GE Healthcare HisPrep™ FF 16/10 Ni-sepharose™ column as follows.

All samples were run on an AKTA™ Pure FPLC system at 4° C. Ni-Sepharose™ columns were first washed with 10 column volumes of ddH2O, then equilibrated with 10 column volumes of lysis buffer. Supernatants from lysed cultures were run over equilibrated columns at a flow rate of 5 mL/min and flowthrough was collected. Columns were washed with 10 column volumes lysis buffer at 5 mL/min, followed by 10 column volumes wash buffer (50 mM Tris-HCl pH 7.4, 0.5 M NaCl, 50 mM imidazole, 0.1% Tween-20, 1 mM DTT) at 5 mL/min. His-tagged proteins were eluted with 4 column volumes elution buffer (50 mM Tris-HCl pH 7.4, 0.5 M NaCl, 250 mM imidazole, 0.1% Tween-20, 1 mM DTT) at 1 mL/min, collecting 5 mL fractions. Samples (20 µL) of lysis supernatant, flowthrough, washes, and elution fractions were mixed with 10 mL of 3X SDS sample loading buffer (187.5 mM Tris-HCl (pH 6.8), 6% (w/v) SDS, 30% [v/v] glycerol, 150 mM DTT, 0.03% (w/v) bromophenol blue, 2% [v/v] betamercaptoethanol) and boiled at 95° C. for 5 mins. Boiled samples (15 mL) were resolved on standard SDS-polyacrylamide gels (15%). Bands were visualized with Coomassie Brilliant Blue and destained with a solution of 40% methanol, 10% acetic acid.

Ni-sepharose elution fractions containing RBD protein were pooled and dialyzed at 4° C. for 5 hours in 1 L of IEX loading buffer (50 mM HEPES pH 8.0, 10 mM NaCl, 1 mM DTT), then overnight in 2 L of fresh IEX loading buffer. The dialyzed sample was then loaded onto a 5 mL SP HP HiTrap™ column at 0.5 mL/min and flowthrough was collected. Columns were washed with 10 column volumes IEX wash buffer (50 mM HEPES pH 8.0, 25 mM NaCl, 1 mM DTT) at 2 mL/min. Bound proteins were eluted with IEX elution buffer (50 mM HEPES pH 8.0, 500 mM NaCl, 1 mM DTT) at 1 mL/min, collecting 1 mL fractions. Samples were analyzed on SDS-polyacrylamide gels as above.

In some cases, Ni-sepharose elution fractions containing RBD protein were pooled and diluted with 4.5 volumes of lysis buffer without NaCl to give a final concentration of ~150 mM NaCl. The sample was then loaded onto a 5 mL Q HP HiTrap™ column at 0.5 mL/min and flowthrough was collected. The flow-through was dialyzed at 4° C. overnight in 2 L of fresh lysis buffer. Columns were washed with 10 column volumes lysis buffer at 2 mL/min. Bound proteins were eluted as above into a small volume (< 5 ml), and the peak was run over a 120 ml Superdex™ HL 75 size exclusion column equilibrated with Phosphate Buffered Saline (PBS). Samples were analyzed on SDS-polyacrylamide gels as above.

IEX elution fractions containing RBD protein were pooled, concentrated using Pierce centrifugal protein concentrators (10 kDa cutoff), and loaded onto a Superdex™ 200 Increase 10/300 GL column (24 mL bed volume) followed by IEX loading budder at 0.5 mL/min. Flowthrough was collected in 0.5 mL fractions. Samples were analyzed on SDS-polyacrylamide gels as above. Elution fractions containing RBD protein were pooled, then concentrated and buffer exchanged with PBS using Pierce centrifugal protein concentrators (10 kDa cut-off).

### Protein Extraction and Purification of SARS-CoV-2 Nucleocapsid

Phaeodactylum tricornutum cultures (5 L) were harvested during stationary growth phase and pelleted at 6000×g for 15 mins at 4° C. Cell pellets were weighed and resuspended 3-4 mL/g of buffer. Cells were then manually dispersed in a Dounce homogenizer and lysed with an Avestin™ C3; one pass at no pressure and 2 passes at 20000-25000 psi. Lysates were centrifuged at 100000 x g for 1 hour at 4° C., then clarified by passage through Whatman™ 6 filter paper followed by 0.45-micron filters. All operations performed on ice. Supernatants were collected in a new tube and stored on ice before purification using a 20 mL GE Healthcare HisPrep FF 16/10 Ni-sepharose column as follows.

All samples were run on an AKTA™ Pure FPLC system at 4° C. Ni-sepharose columns were first washed with 10 column volumes of ddH₂O, then equilibrated with 10 column volumes of lysis buffer. Supernatants from lysed cultures were run over equilibrated columns at a flow rate of 5 mL min⁻¹ and flowthrough was collected. Columns were washed with 10 column volumes lysis buffer at 5 mL min⁻¹, followed by 10 column volumes wash buffer (50 mM Tris-HCl pH 7.4, 0.5 M NaCl, 10 mM imidazole, 2 M Urea, 0.1% Tween-20, 1 mM DTT) at 5 mL min⁻¹. The column was then equilibrated back to 0 mM urea by a further 10 column volumes of loading buffer. His-tagged proteins were eluted with 4 column volumes elution buffer (50 mM Tris-HCl pH 7.4, 0.5 M NaCl, 300 mM imidazole, 0.1% Tween-20, 1 mM DTT) at 1 mL min⁻¹, collecting 5 mL fractions. Samples (20 µL) of lysis supernatant, flowthrough, washes, and elution fractions were mixed with 10 µL of 3× SDS sample loading buffer (187.5 mM Tris-HCl (pH 6.8), 6% (w/v) SDS, 30% [v/v] glycerol, 150 mM DTT, 0.03% (w/v) bromophenol blue, 2% [v/v] β-mercaptoethanol) and boiled at 95° C. for 5 mins. Boiled samples (15 µL) were resolved on standard SDS-polyacrylamide gels (15%). Bands were visualized with Coomassie Brilliant Blue and destained with a solution of 40% methanol, 10% acetic acid.

Ni-sepharose elution fractions containing Nucleocapsid protein were pooled and diluted with 4.5 volumes of lysis buffer without NaCl to give a final concentration of ~150 mM NaCl. The sample was then loaded onto a 5 mL Q HP HiTrap™ column at 0.5 mL min-1 and flowthrough was collected. The flow-through was dialyzed at 4° C. overnight in 2 L of fresh lysis buffer. Columns were washed with 10 column volumes lysis buffer with 20 mM imidazole at 2 mL min-1 . Bound proteins were eluted as above into a small volume (< 5 ml), diluted to 10 mM NaCl with salt free lysis buffer and loaded onto a 1 ml SP HP HiTrap™ column at 0.5 mL min-1 equilibrated with lysis buffer with 10 mM NaCl. The Nucleocapsid protein was eluted using 1 M NaCl and the and the peak was collected, and the buffer was exchanged for PBS. Samples were analyzed on SDS-polyacrylamide gels as above.

### Western Blots

Samples were resolved on standard SDS-polyacrylamide gels (15%) and electroblotted to a polyvinylidene difluoride (PVDF) membrane using a Trans-Blot Turbo™ Transfer System (BioRad, Hercules, CA, USA). Membranes were incubated for 1 hour in blocking solution (3% bovine serum albumin (BSA), 0.1% Tween-20, 1X TBS) before adding anti-RBD primary antibody (SinoBiological, 40592-T62) at 1:100 final dilution, or anti-GFP primary antibody (Invitrogen, A-6455) at 1:2500 final dilution, or anti-His primary antibody (Invitrogen, MA1-21315) at a 1:1000 final dilution. Membranes were incubated overnight at 4° C., washed for 3 × 10 mins in washing solution (1% BSA, 0.1% Tween-20, 1X TBS), then incubated with anti-rabbit (Sigma, GENA9340) or anti-mouse (Amersham, NA931) horseradish peroxidase-linked secondary antibody for 2 h at 1:5000 final dilution in washing solution. Membranes were then washed in 1X TBS with 0.1% Tween-20 for 3 × 10 mins, followed by one wash for 10 mins in 1X TBS. Blots were developed using Clarity ECL™ Western Blotting Substrate (BioRad) following the manufacturer’s instructions and imaged with a ChemiDoc™ XRS+ System (Bio-Rad). To evaluate the secretion of algae-RBD, a P. tricornutum culture (50 mL) was harvested during stationary growth phase and pelleted at 3,000 × g for 10 mins at 4° C. The supernatant was filtered through a 0.2 µm filter and concentrated to 150 µL using a Pierce centrifugal protein concentrator (10 kDa cutoff). Concentrated supernatant (20 µL) was mixed with 10 µL of 3× SDS sample loading buffer (187.5 mM Tris-HCl (pH 6.8), 6% (w/v) SDS, 30% [v/v] glycerol, 150 mM DTT, 0.03% (w/v) bromophenol blue, 2% [v/v] β-mercaptoethanol) and boiled at 95° C. for 5 mins. Boiled sample (15 µL) was resolved on a standard SDS-polyacrylamide gel (15%). Bands were visualized with Coomassie Brilliant Blue and destained with a solution of 40% methanol, 10% acetic acid. For western blots, 0.5 µL of boiled sample was resolved on a standard SDS-polyacrylamide gel (15%).

### Mass Spectrometry

Protein samples were resolved on a 15% SDS-PAGE gel. Bands were visualized with Coomassie Brilliant Blue and destained with a solution of 40% methanol, 10% acetic acid. Bands were excised and placed in 1.5 mL microfuge tubes with 500 µL of 1% acetic acid. Mass spectrometry analysis and peptide identification by ms/ms was performed at the SPARC BioCentre, Sick Kids Hospital, University of Toronto. Peptide data was downloaded from the SPARC BioCentre server and analyzed by the Scaffold software package using a FASTA file of the P. tricornutum and SARS-CoV-2 proteomes. The protein threshold was set at 90% and the peptide threshold at a 1% false discovery rate.

### Gold Conjugation of Algae-RBD

For the preparation of gold labelled algae-RBD, 40 nm colloidal gold particles (International Point of Care Inc) were adjusted to a pH of 9.45 (±0.15) using 1.0 M potassium carbonate buffer. The pH-adjusted gold was then diluted in ultrafiltrate H₂O to an optical density (OD) of 1.0 (peak absorbance observed between wavelength 350 to 540 nm). Purified algae-RBD (0.213 mg/mL) was conjugated by passive adsorption to gold at a final concentration of 6 µg/mL algae-RBD per mL of OD 1.0 gold. After incubation at ambient temperature (22-25° C.) for 45 minutes, a solution of 10% bovine serum albumin and 1% polyethylene glycol comprising 5% of the total conjugate volume was added and incubated at ambient temperature for 30 mins under gentle mixing. After incubation the gold conjugate solution was centrifuged at 12,000 x g for 30 mins. Without disturbing the pellet, the resulting supernatant was removed and discarded. The pellet was then resuspended in 20 mM Tris-HCl (pH 9.25) representing 80% of the initial conjugate volume. This process was repeated in two consecutive centrifugation and resuspension steps. A final centrifugation step was performed and the supernatant discarded. The pellet was resuspended in the remaining supernatant. The RBD labelled gold was then stored overnight at 2-8° C. to observe aggregation. Following overnight incubation, algae-RBD gold conjugate was briefly sonicated in a water bath to disperse any gold aggregates. A sample of the gold conjugate was then diluted 1/50 in Tris-HCl pH 9.25, and the absorbance peak was measured. The final OD of the gold conjugate was determined to be 12.8.

### Preparation and Testing of LFA Devices

A commercially available qualitative lateral flow COVID-19 serology test (Lumivi) was adapted to evaluate the algae-RBD that was lyophilized onto polyester pads. Nitrocellulose membrane (Millipore) was striped at a test line location with an anti-human IgG antibody (BiosPacific Inc.) and at a control line location with a Goat anti-Rabbit polyclonal antibody (Cedarlane Inc.). Following lamination onto adhesive lined polyester backing cards, the resulting cards were cut into cut into 5.5 mm strips and placed into the Lumivi commercial housing, packaged in foil pouches with desiccant and stored at ambient temperature for subsequent testing. For comparison purposes a SARs-CoV-2 Spike protein RBD domain expressed in a human cell line (DAGC174 Creative Diagnostics) was similarly conjugated to colloidal gold as described previously and evaluated in the adapted lateral flow assay. A 15 µL aliquot each test sample (plasma) was applied to the device sample well followed by three drops of 0.1 M PBS/Tween running buffer (approximately 150 L). After a 15-minute incubation at ambient temperature the test was visually interpreted for the presence of purple/red lines at both the Test and Control marker areas within the devices read window. The devices were scanned with an optical reader (i-Lynx™) and values of 0.055 reflectance units were considered to be visually detectable by untrained operators and are positive. Values below 0.055 reflectance units are scored as negative. A positive control line (indicating proper sample flow within the prototype device) was required before device interpretation could be made.

### Example 2: Construction of SARS-CoV-2 RBD Expression Plasmid, Conjugation and Stable Maintenance in P. Tricornutum

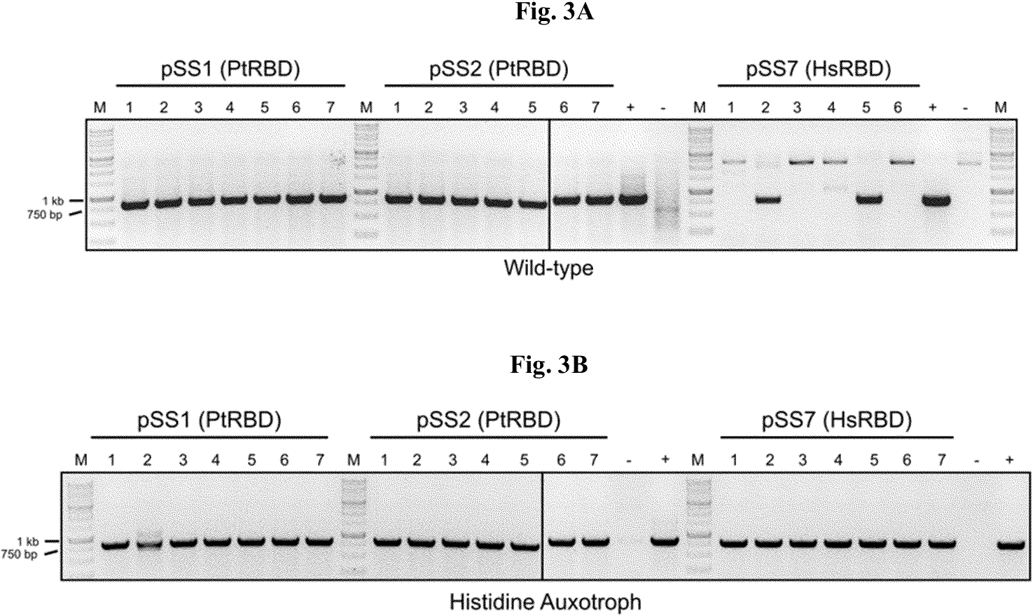

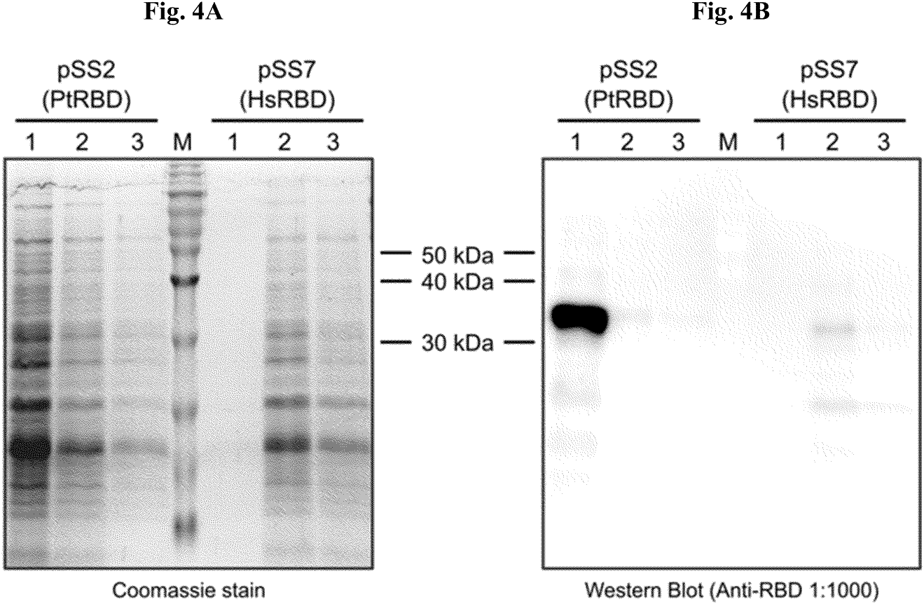

The coding region for the RBD of the SARS-CoV-2 spike protein with an added C-terminal 6X-histidine tag and TEV protease site was cloned into the E. coli- S. cerevisiae-P. tricornutum plasmid vector pDMI2 (see plasmid map shown in FIG. 2). In the first set of plasmids (pSS1 and pSS2), the RBD coding region was codon-optimized for P. tricornutum (PtRBD) and targeted for secretion using the promoter and secretory signal from the P. tricornutum HASP1 gene (highly abundant secreted protein 1) (Erdene-Ochir et al., 2019) (FIG. 2). pSS1 and pSS2 differ in nucleotide polymorphisms in the promoter that are present in different alleles of HASP1. Another plasmid (pSS7) used a human codon optimized RBD coding region (HsRBD) with the SARS-CoV-2 spike protein secretory signal. Plasmids were introduced into wild type P. tricornutum or a histidine auxotroph strain (Slattery et al., 2020) from E. coli by conjugation. After isolation of single P. tricornutum clones, retention of the RBD coding region was assessed after 28 days growth in liquid culture. As shown in FIGS. 3A and 3B, diagnostic PCRs on individual clones for the PtRBD coding region indicated no rearrangements in the RBD coding region in the wild type or histidine auxotroph strains of P. tricornutum, whereas the HsRBD appeared stable in only the histidine auxotroph strain. Transcription of the RBD was confirmed by RNAseq for 4 clones of pSS2. Histidine auxotroph strains harbouring three clones of pSS2 (PtRBD) or pSS7 (HsRBD) plasmids were expanded in liquid culture and RBD expression examined by Western blotting using a polyclonal anti-RBD antibody (FIGS. 4A and 4B). One strain of pSS2 revealed robust RBD expression (PtRBD-1) and was chosen for lonf-term growth experiments over 7 months. At 7 months, larger-scale bioreactors (20-L) were seeded with the PtRBD-1 strain, and samples taken for diagnostic PCR and Western blotting for RBD expression. Of the bioreactor cultures sampled for diagnostic PCR, the RBD was present in 10 of the 12 samples (FIG. 5). Western blotting of whole cell lysates revealed robust RBD expression in 8 of 14 bioreactors, and weaker or undetectable expression in the remaining bioreactors (FIG. 5).

We also constructed HASP1-regulated RBD plasmids with glutathione S-transferase (GST), 10X-histidine, and IgG1-Fc purification tags as N- or C-terminal fusions to determine stability and expression levels in small-scale laboratory cultures. In each case, Western blotting with a polyclonal anti-RBD antibody revealed expression of the RBD fusions, although at varying levels (data not shown). Together, these data show that plasmids with RBD coding regions optimized for expression in P. tricornutum can be maintained in laboratory scale or larger scale cultures for at least 7 months, that robust RBD expression can be detected by RNAseq and by a polyclonal anti-RBD antibody, and that 4 different purification tags are compatible with RBD expression, providing different strategies for purification.

### Example 3: Limiting Phosphate Induces Expression from the HASP1 Promoter

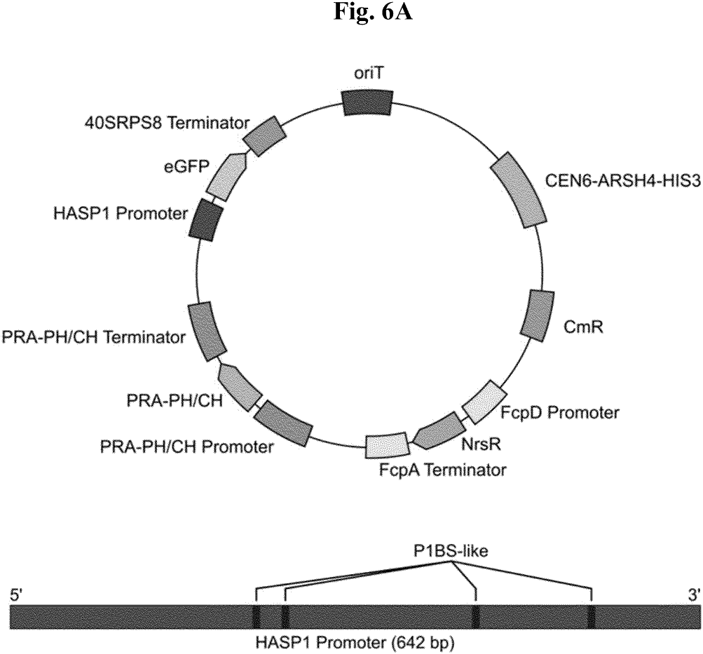

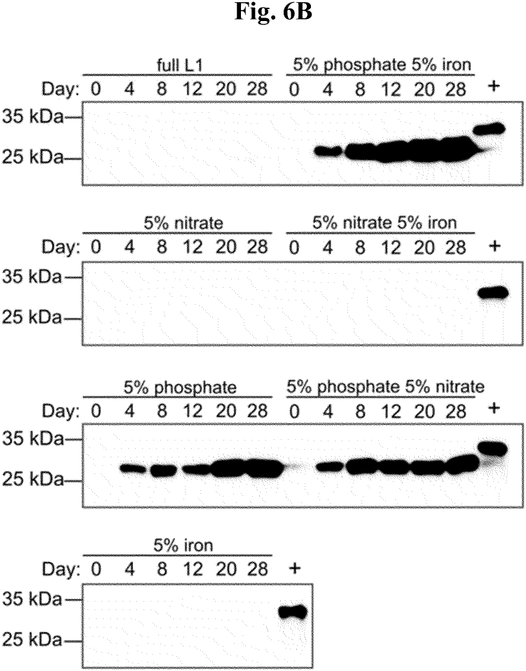

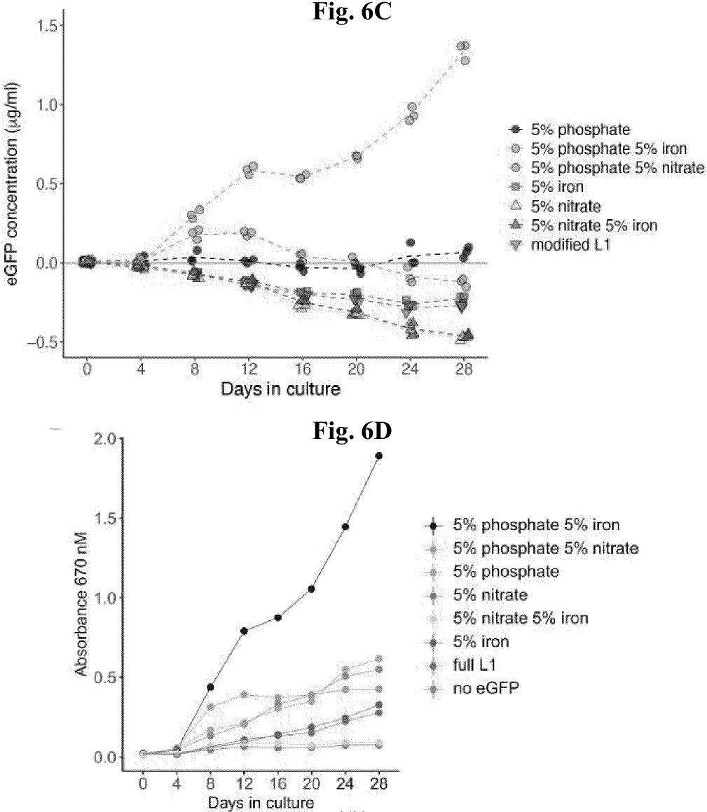

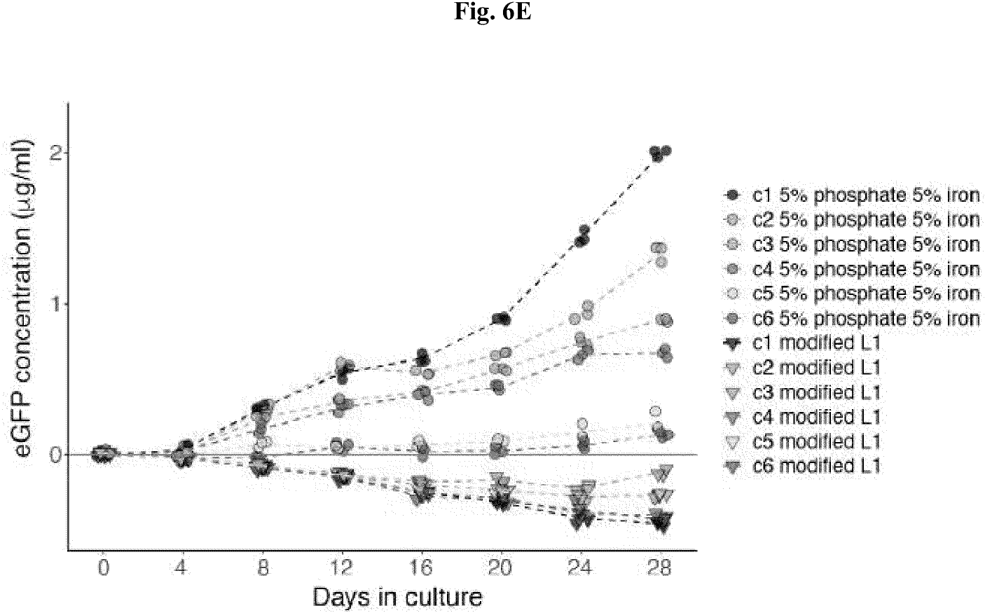

In an effort to maximize production of the RBD, we noted that the HASP1 promoter sequence may contain a number of potential regulatory elements predicted via in silico analyses (Erdene-Ochir et al., 2019), which may affect expression levels based on environmental conditions (Dell’Aguila et al. 2020). We thus constructed a plasmid (pSS10) where the coding region for enhanced green fluorescent protein (eGFP) was cloned downstream of a 650-bp sequence containing the HASP1 promoter region (FIG. 6A). Stable P. tricornutum clones harbouring pSS10 were selected, grown in media containing high concentrations of phosphate (100%; about 362 µM; about 34 ppm) and then diluted into test tubes containing media with phosphate at 5% of normal media levels (5%; about 18.1%; about 1.7 ppm), or with reduced amounts of other media constituents. We examined eGFP expression by Western blotting of whole cell lysates of cultures sampled at different days post inoculation and observed robust expression of eGFP in media that was reduced in phosphate alone (“5% phosphate”), phosphate and iron (“5% phosphate 5% iron”), and phosphate and nitrate (“5% phosphate 5% nitrate”) as early as day 4 in the time course (FIG. 6B). The HASP1-eGFP induction by low phosphate media was replicated with an independent isolate of pSS10 in P. tricornutum (data not shown). As also shown in FIG. 6B, no eGFP expression was detected in strains grown in media reduced in nitrate alone (“5% nitrate”), iron (“5% iron”), or both nitrate and iron (“5% nitrate 5% iron”).

Interestingly, secreted eGFP was evident after 8 days in supernatants of media with 5% phosphate and 5% iron, and continued to increase over the time course of the experiment (FIG. 6C). In contrast, no secreted eGFP was observed in supernatants of L1 media (Full L1). Limiting phosphate in combination with nitrate, nitrate alone, or nitrate in combination with iron, did not stimulate eGFP secretion to the supernatant (FIG. 6C). The differences in secreted eGFP for these clones many be related to mutations that were identified by whole-plasmid sequencing that likely occurred during conjugation from E. coli to P. tricornutum. Intriguingly, growth curves of P. tricornutum in 5% phosphate and 5% iron revealed no difference to growth in full L1 media, whereas strains grown in media with reductions in other constituents had much slower rates (FIG. 6D). Limiting phosphate in combination with nitrate, nitrate alone, iron alone, or nitrate in combination with iron, did not stimulate eGFP secretion to the supernatant. eGFP secretion to the culture supernatant was observed with 5 other pSS10 clones in 5% phosphate 5% iron media, but not in modified L1 media (FIG. 6E). Collectively, these data show that eGFP secretion was robust in media with reduced phosphate and iron, whereas limiting phosphate alone was sufficient to stimulate eGFP expression from the HASP1 promoter.

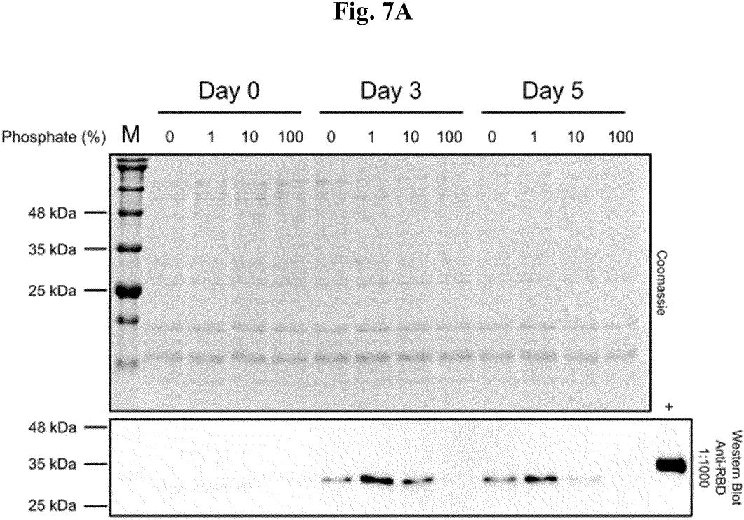

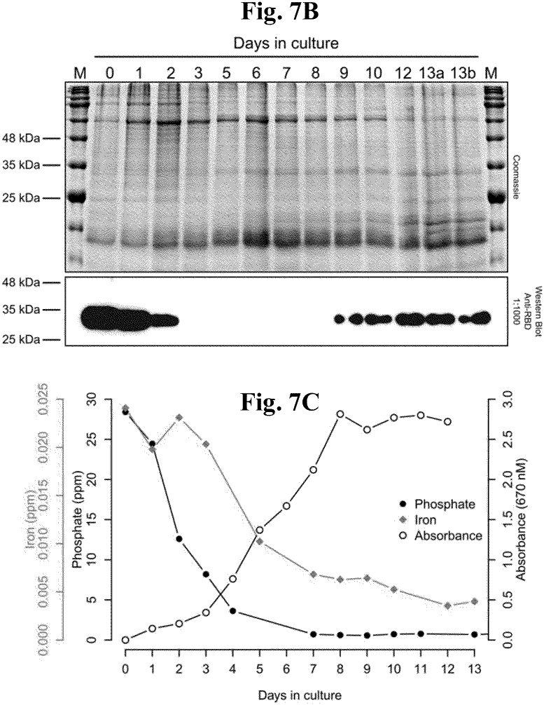

Encouraged by this result, we next tested whether expression of the RBD from the HASP1 promoter was also regulated by limiting phosphate and/or iron. We first tested this by diluting a stationary phase culture of pSS2 into small-scale cultures (10 mL) with fresh L1 media containing different concentrations of added phosphate (0%, 1%, 10% and 100%; corresponding to about 0, 0.34, 3.4, and 34 ppm, respectively). By day 3, RBD expression as observed by Western blotting of whole cell extracts in all cultures except those containing 100% phosphate (FIG. 7A), indicating that the HASP1 promoter was responsive to reduced levels of phosphate with maximal induction between 1 and 10% phosphate.

We next scaled expression to 5 L to facilitate monitoring of growth rate, RBD expression, phosphate and iron concentration in a bioreactor containing media with our standard L1 media (100% phosphate, about 34 ppm phosphate). For this experiment, we seeded the bioreactor with a culture in a late stationary phase that showed high levels of RBD expression (FIG. 7B, lane 0). As shown in FIG. 7B, RBD levels as indicated by Western blots of whole cell lysates were high at early time points (days 1 and 2), not detected at days 3-8, and then visibly induced by day 9. Reduction of phosphate in the media to below about 0.5 or 1 ppm correlated with the observed maximal RBD expression (FIG. 7C). A similar reduction in iron was also observed (FIG. 7C). Taken together, these data show that the HASP1 promoter can be induced or repressed based on phosphate levels in the culture media, with < 1 ppm phosphate inducing expression. This result also explains why we found that in our modified L1 media, which contains about 34 ppm phosphate or 10 times more than other studies (Erdene-Ochir, 2019), HASP1-driven expression of the RBD was highest in late stationary phase when phosphate depletion to levels necessary for induction occurred, and why our western blot data shows full RBD repression.

### Example 4: Purification of PtRBD and Effect of the TEV Protease Site on Expression

Our initial protein purification strategy was based on secretion of the expressed SARS-CoV-2 proteins into algae culture supernatant using the HASP1 secretory signal and with the endoplasmic retention signal intact or mutated to alanine residues. However, we found no evidence of secretion of the expressed RBD in culture supernatants, either in small scale cultures or larger bioreactors. Lack of RBD secretion was not due to the HASP1 promoter and secretory peptide, as we observed robust secretion of the HASP1-eGFP construct (FIG. 6C). We also found no evidence of RBD secretion in culture supernatants of strains harbouring pSS7 that expresses a human codon-optimized RBD driven by the P. tricornutum 40SRPS8 constitutive promoter and the native SARS-CoV-2 spike protein secretion signal. Moreover, plasmids expressing algae codon-optimized versions of the full-length spike protein (pSS3-pSS6) also had poor expression levels and we found significant proteolysis of the spike protein as evidenced by mass spectrometry analyses of concentrated cell-free media (data not shown).

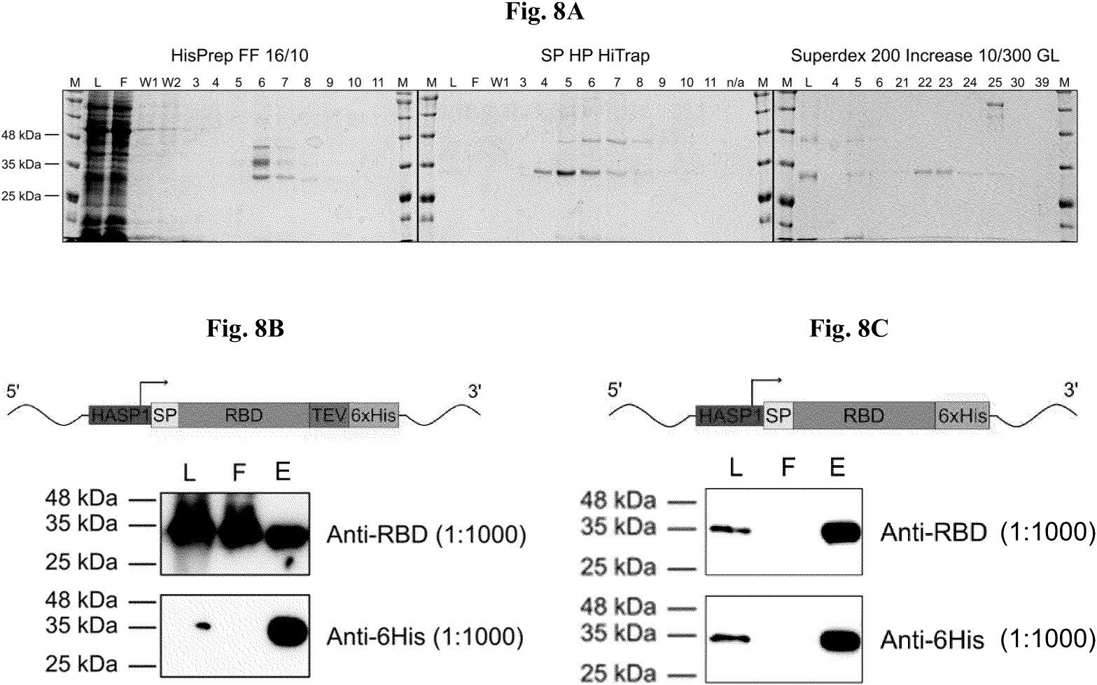

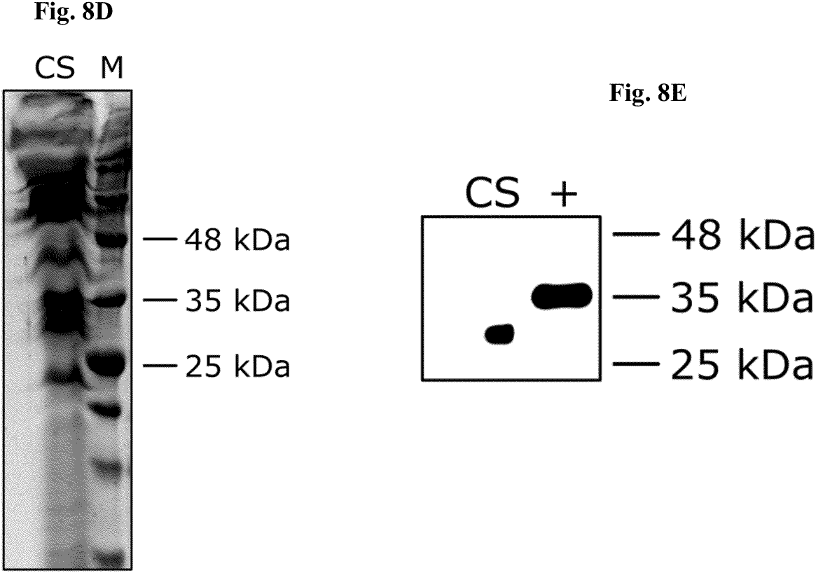

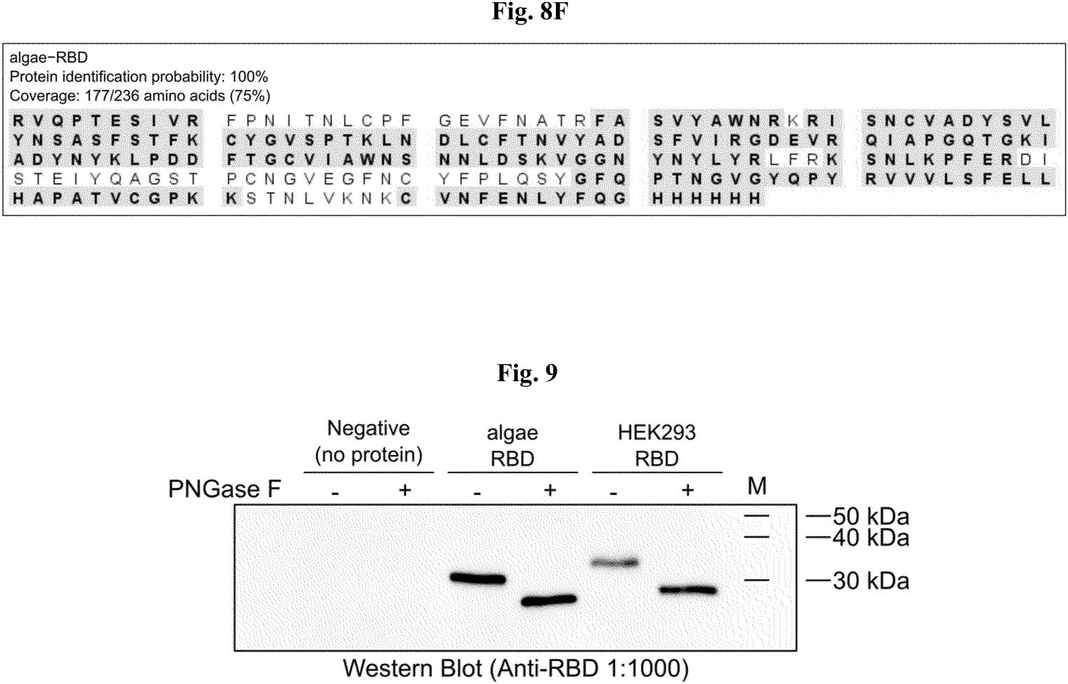

We focused on purifying the 6X-histidine tagged RBD from whole-cell extracts of P. tricornutum using a combination of metal affinity, ion exchange and gel filtration chromatography (FIG. 8A). From 5-L bioreactor runs, we achieved RBD yields of 28-34 µg of RBD. During repeated purification runs, we noted that a significant portion of the expressed RBD was present in the column flow through (~90-95%) (FIG. 8B). Purification under denaturing conditions (6 M urea) did not improve binding to the metal affinity column (data not shown). Interestingly, Western blotting of the column load, lysate, flow through and eluate with anti-HisTag and anti-RBD antibodies showed that the majority of the RBD present in the load and flow through lacked a 6X-histidine tag, providing an explanation for why most of the expressed RBD did not bind the metal affinity column.

We considered the possibility that the TEV protease site in the algae-RBD construct was a potential substrate for a P. tricornutum protease, thus causing loss of the C-terminal 6X-histidine tag from expressed algae-RBD. Surprisingly, when we deleted the TEV protease site from the construct (pSS72), but retained the C-terminal 6X-histidine tag, the majority of the protein was present in the eluate and not in the flow-through (FIG. 8C), suggesting that the presence of the TEV protease site caused loss of the 6X-histidine tag. Furthermore, RBD constructs lacking a TEV protease site were also present in the supernatant of culture P. tricornutum, suggesting that these RBD constructs were more readily secreted, in comparison to RBD constructs having a TEV protease site (FIGS. 8D and 8E). The finding of enhancing secretion of recombinantly-expressed proteins in P. tricornutum by removal of a TEV protease cleavage site was surprising and significant in that previous studies expressing RBD in algae, such as in Berndt et al., 2021 using green algae (Chlamydomonas reinhardtii), were unsuccessful in secreting RBD.

The identity of the purified algae-RBD was confirmed by mass spectrometry protein identification (FIG. 8F) and by Western blotting with polyclonal anti-RBD antibody (FIG. 9). We noted that the apparent molecular weight of the algae-RBD was lower than that of RBD produced in mammalian cell lines (HEK293) (FIG. 9).

We tested purified algae-RBD for the presence of N-linked glycosylated residues by treatment with the endoglycosidase PNGase F that cleaves between the innermost N-acetylglucosamine and asparagine residues. As shown in FIG. 9, treatment with PNGase F reduced the apparent size of the algae-RBD. A similar treatment of commercially available RBD purified from mammalian cell lines (HEK293-RBD) also reduced the apparent size of the RBD (FIG. 9). The algae-RBD and mammalian RBD do not differ in primary sequence of the recombinant constructs and thus the slight difference in the apparent size of the pre- and post-treated algae-RBD versus mammalian RBD could be due to post-translational modifications other than N-linked glycosylation, including reported O-linked glycosylation of the SARS-CoV-2 spike protein (Tian et al., 2021). These data, however, are consistent with both RBD preparations containing N-linked glycosylated residues.

### Example 5: Recombinant RBD Expressed in P. Tricornutum Competes for Binding to Human ACE2 Receptor and is Recognized by Anti-Spike Protein Antibodies From Patient Samples

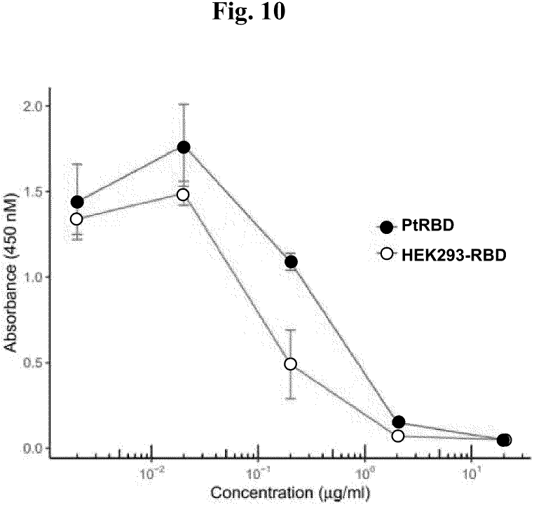

The biological activity of the PtRBD was analyzed by performing an in vitro assay where addition of RBD is used to competitively inhibit binding of an Fc-tagged mammalian purified RBD to an immobilized ACE2 extracellular domain (FIG. 10). For this experiment, we used both the PtRBD and a commercially available RBD purified from HEK293 cells (HEK293-RBD). We found very similar inhibition profiles for both the PtRBD and HEK293-RBD. Together, these data show that biologically active PtRBD that contains N-linked glycosylations can be purified from whole cell extracts of P. tricornutum.

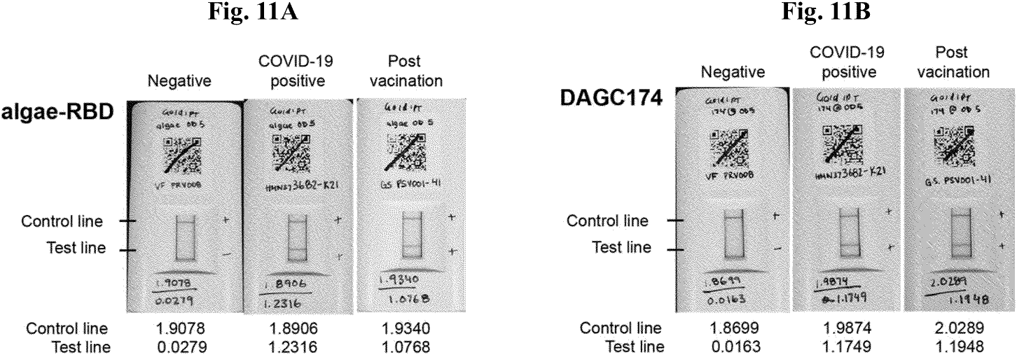

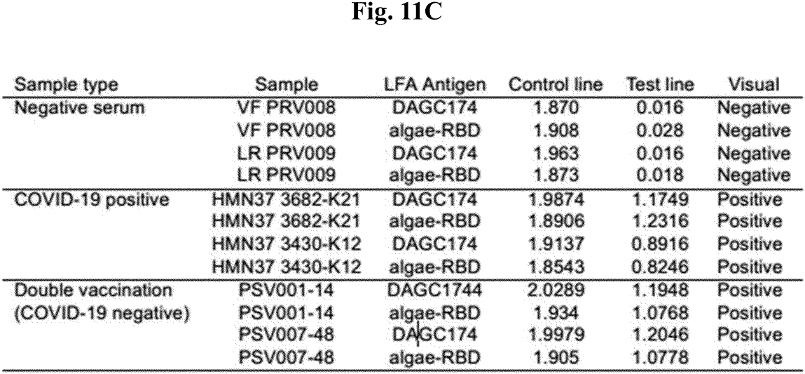

To demonstrate the practical application of the PtRBD in a serological test that would commonly be used to determine immune response to SARS-CoV-2 infection, or immune response post-vaccination, we conjugated the PtRBD to gold beads that were applied to a lateral flow assay (LFA) device. As shown in FIGS. 11A-11C, the algae-RBD LFA was able to detect the presence of anti-RBD IgG antibodies in serum from two sources; from patients previously infected with SARS-CoV-2 as determined by PCR, and from patients confirmed COVID-19 negative by PCR and subsequently immunized with two doses of the Pfizer-BioNTech BNT162b2 vaccine. Importantly, the sensitivity of the LFA with the algae-RBD was equivalent to the LFA with the commercially available RBD antigen (DAGC174, Creative Diagnostics) produced in mammalian cells. No reactivity was observed for either antigen when tested against serum negative for COVID-19 by PCR testing.

In summary, the data presented herein demonstrate that serologically active recombinant RBD of the SARS-CoV-2 Spike protein can be expressed and purified from P. tricornutum. The RBD expressed in P. tricornutum was reactive with anti-Spike protein antibodies, competitively inhibited binding of mammalian-expressed RBD to the ACE2 extracellular domain, and was able to detect anti-RBD IgG antibodies from patient serum in an LFA device.

### Example 6: Expression of SARS-CoV-2 Nucleocapsid in P. Tricornutum (PtNC) and Purification

Next, SARS-CoV-2 nucleocapsid protein was expressed in P. tricornutum in the expression plasmid pSS40 (SEQ ID NO: 21). P. tricornutum was then maintained as described in Examples 2 and 3 for PtRBD, unless otherwise specified.

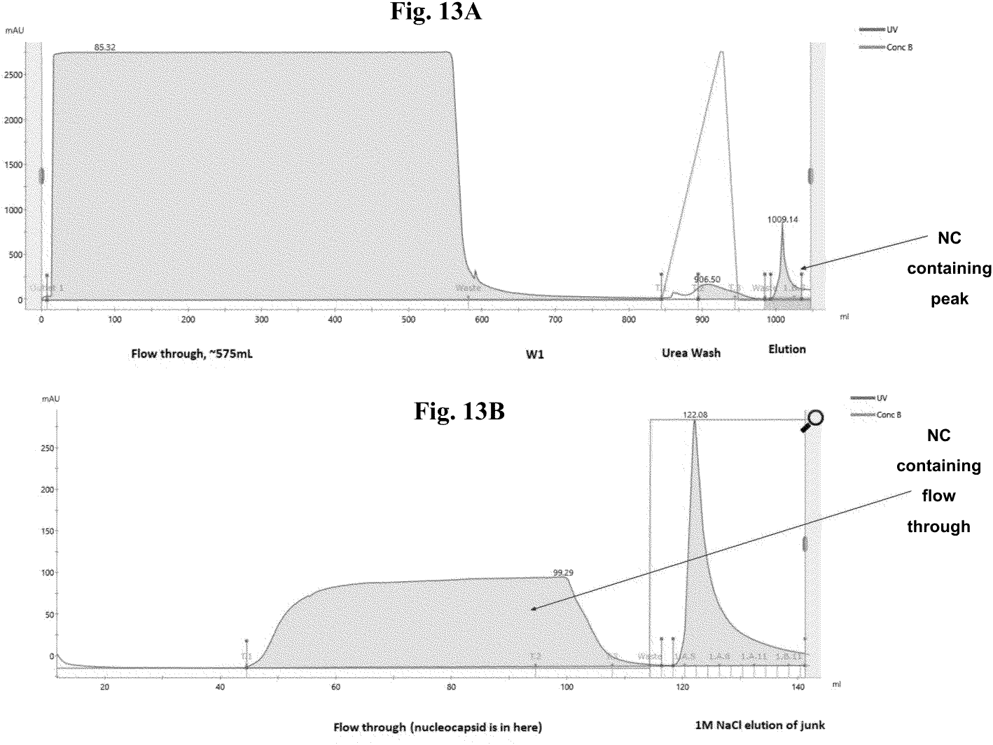

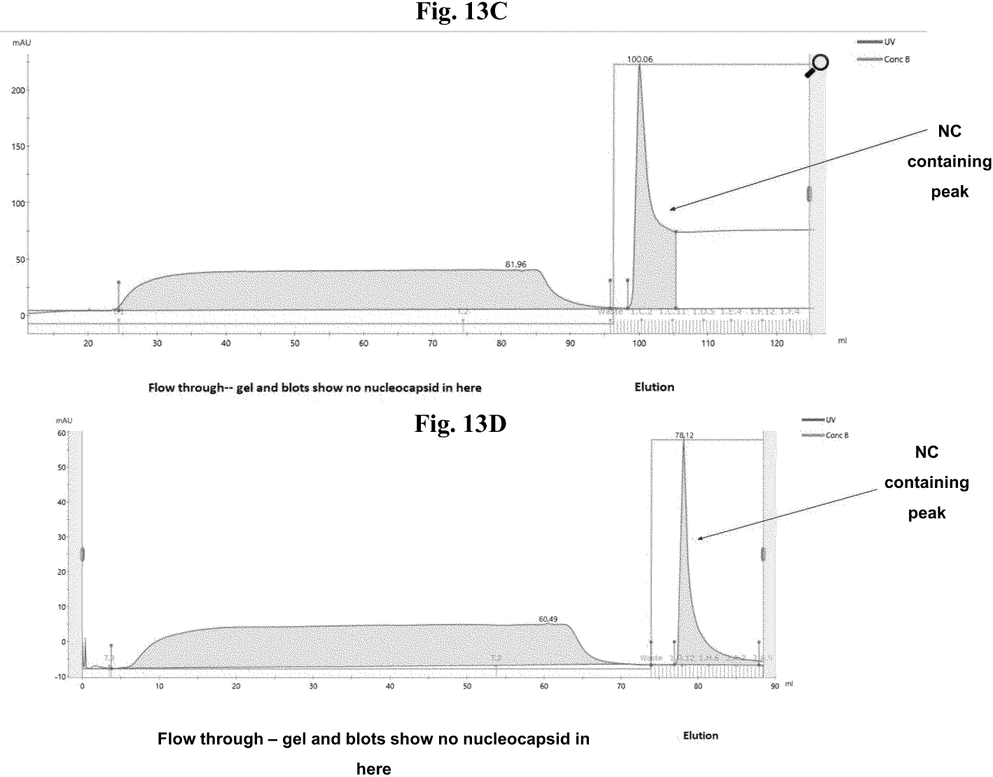

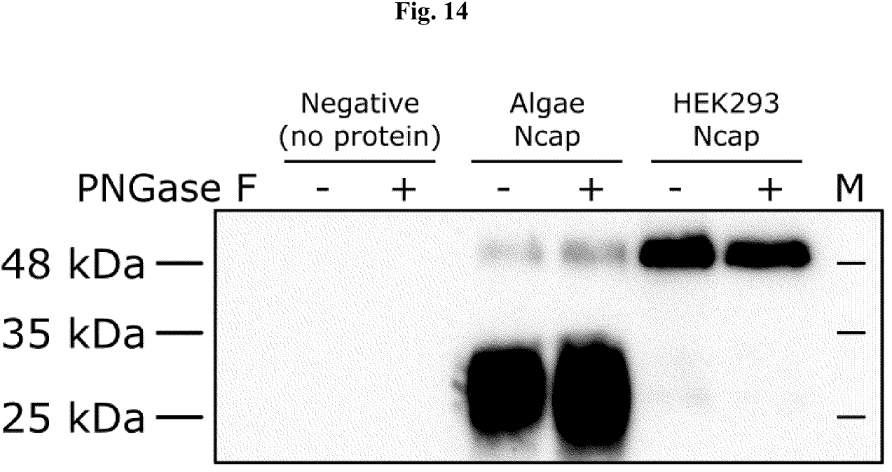

Extraction and purification of PtNC was then done according to methods described in Example 1. FIGS. 12A-12C shows the purification of nucleocapsid protein (NC) expressed from pSS40 plasmid expressed in P. tricornutum (PtNC). As shown by the Coomassie stain (FIG. 12A) and Western blots using an anti-nucleocapsid polyclonal antibody (FIG. 12B) or an anti-6His antibody (FIG. 12C), Nucleocapsid protein was successfully extracted and purified from P. tricornutum (black arrows). Cross-reacting species (white arrow, CRP), which were slightly smaller than the full-length nucleocapsid (black arrow, NC), were observed but that do not contain multiple histidine residues since they were not recognized by anti-6His antibodies. FIGS. 13A-13D shows the absorbance of the purification steps from the AKTA™ Pure FPLC system run with the columns indicated in the methods of Example 1. These are annotated as to the location of the NC protein at each step in the protocol.

Next glycosylation of PtNC was assessed. Although native SARS-CoV-2 nucleocapsid protein is not glycosylated, we wanted to confirm that purified PtNC was also not glycosylated. As shown in FIG. 14 using the same PNGase F assay as for PtRBD (FIG. 9), band sizes of PtNC were unaffected upon treatment with PNGase F, indicating that PtNC was not glycosylated. The predominant recombinant PtNC protein expressed and purified were N-terminally truncated, resulting in a species that migrated from at about 35 kDa to less than 25 kDa by SDS-PAGE (FIGS. 12A-12C and 14). Analysis by mass spectroscopy confirmed that the purified PtNC N-terminally truncated protein comprised at least residues 226-443 of SEQ ID NO: 23. Nevertheless, the N-terminally truncated PtNC protein was still recognizable by anti-nucleocapsid antibodies (FIGS. 12A-12C and 14).

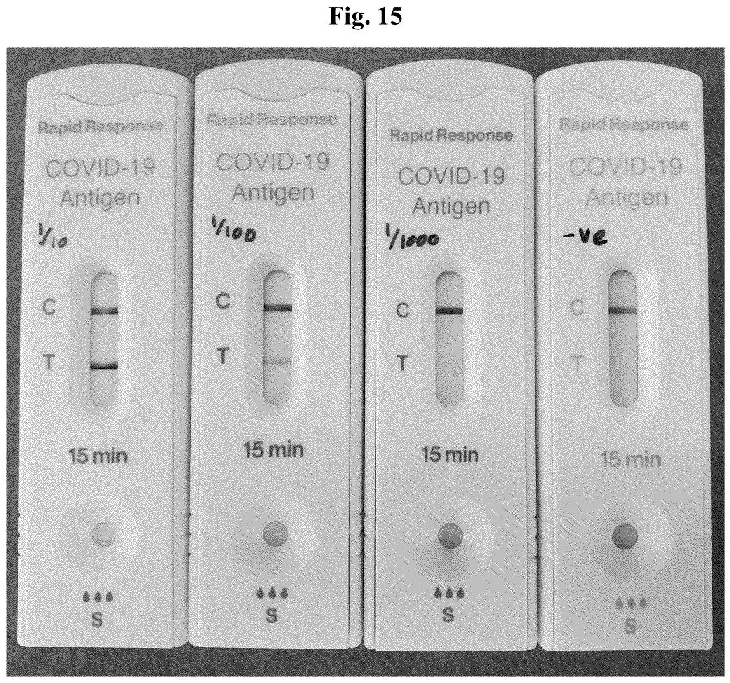

To demonstrate the practical application of the PtNC in a potential serological test that would commonly be used to determine immune response to SARS-CoV-2 infection, or immune response post-vaccination, as well as to distinguish patients with active SARS-CoV-2 infection from vaccinated patients, we conjugated the PtNC to gold beads that were applied to a lateral flow assay (LFA) device, as done for PtRBD. As shown in FIG. 15, the algae-NC LFA was able to detect the presence of anti-NC polyclonal antibodies at ⅒ and 1/100 dilutions. At 1/1000, the positive band was very faint but present.

In summary, the data presented herein demonstrate that other SARS-CoV-2 proteins, including unglycosylated proteins, can be expressed and purified from P. tricornutum. The NC expressed in P. tricornutum was reactive with anti-NC protein antibodies, and may be potentially used in an LFA device to detect patient serum antibodies.

### Example 7: Discussion

Available data and modelling indicate that the current COVID-19 pandemic will remain a public health issue beyond the current waves of SARS-CoV-2 infection. Moreover, SARS-CoV-2 variants (such as the Delta B.1.617.2 variant) that have enhanced infectivity and/or pathogenicity relative to the parental SARS-CoV-2 strain will likely continue to arise, and it is possible these and other variants will become endemic. Rapid LFAs that utilize recombinantly expressed SARS-CoV-2 antigens are one type of serological test useful for viral exposure monitoring or for determining immune response post-vaccination (Whitman et al., 2020). Widespread use of LFAs will require a scalable source of immunologically reactive viral antigen. Here, we show that the marine diatom P. tricornutum is a viable orthogonal and scalable system for overexpression and purification of the SARS-CoV-2 RBD and nucleocapsid, and possibly other viral antigens, for use in pandemic diagnostics. In particular, the minimal biocontainment measures, defined growth media and lack of infectivity by mammalian viruses make P. tricornutum an attractive orthogonal system.

Our study focused on expressing the RBD of the SARS-CoV-2 spike protein, as well as the nucleocapsid protein, in algae using plasmid-based expression systems that allowed us to test a number of promoter-RBD combinations, one of which was the promoter from the HASP1 gene. Interestingly, we found that the HASP1 promoter was responsive to inorganic phosphate levels in the culture media, with expression being repressed by higher phosphate levels. Thus, the HASP1 promoter is responsive to limiting phosphate conditions and adds to the inducible expression toolbox of P. tricornutum that is currently based on the nitrate reductase promoter (Niu et al., 2013; Chu et al., 2016; Adler-Agnon et al., 2018) and alkaline phosphatase promoter (Lin et al., 2017). Indeed, our data suggest a number of strategies for phosphate- and iron-regulated expression that would be applicable for toxic proteins or for timing expression for particular growth stages.

We found that overexpressed RBD constructs with a TEV cleavage site was not secreted and was retained intracellularly, while RBD constructs lacking a TEV cleavage site were readily secreted, contrary to observations in Chlamydomonas where the RBD was found to be retained in the endoplasmic reticulum (ER) (Berndt et al., 2021). Deleting the TEV protease site from the construct resulted in a substantial increase in yield with no protein visible by western blotting in the metal-affinity column flow-through. Without being bound by theory, one interpretation of this result is that P. tricornutum encodes an endogenous protease that recognizes the TEV protease site and subsequent cleavage releases the C-terminal 6X-histidine tag. The C-terminal 6X-histidine tag could also be lost by pre-mature transcription termination, post-transcriptional processing or pre-mature translation termination. The fact that we could recover some expressed algae-RBD-6X-His containing the TEV protease site suggests that, regardless of the mechanism that removes the C-terminal 6X-histidine tag, it is inefficient or restricted to a particular sub-cellular compartment (e.g., the ER or Golgi). It is not clear from other studies with P. tricornutum if similar issues were observed during purification of histidine-tagged proteins, although proteolytic removal of C-terminal tags from overexpressed proteins has been observed in E. coli (Lykkemark et al., 2014). This data also suggest that different tags may be better suited for expression and purification of the algae-RBD. RBD constructs using GST, Fc, or 10X-His tags were successfully purified in Pt, and cleavage of at least the Fc and GST tags from RBD was observed by Western blot (data not shown), which indicates that different tags may be used with RBD and that TEV-mediated protease cleavage by Pt is not only specific to 6X-His.

The RBD that we purified from whole cell extracts possessed N-linked glycosylation suggesting that a portion of expressed RBD is transited through the ER/Golgi apparatus and is either secreted at levels too low to be detected in culture supernatants, or is not secreted at all. Although RBD was found to be recalcitrant to secretion when overexpressed, this is not be the case for all proteins, as we found robust secretion of eGFP with the same HASP1 promoter/secretory peptide combination as used for the RBD studies.

The PtRBD was biologically active in an ACE2 receptor binding inhibition assay and serologically active in a test LFA device where it performed at sensitivities similar to commercially available RBD antigen made in mammalian cell lines. This result demonstrates that P. tricornutum is a viable orthogonal protein expression system to mammalian cell culturing for SARS-CoV-2 and potentially other viral antigens. P. tricornutum, and other algae, have a number of advantages over mammalian expression systems, foremost being minimal biocontainment requirements, a defined growth media that lacks potentially cross-reactive antigens present in serum needed for cell culturing, insensitivity to infection by mammalian viruses, and a scalability (>10,000 L) that exceeds many mammalian bioreactors. Moreover, the P. tricornutum histidine auxotroph that we used in our experiments alleviates the need for antibiotic selection of plasmids, further reducing the complexity and cost of growth media. An additional advantage of plasmid-based expression systems is the ability to screen through large numbers of potential expression constructs in response to emerging pandemic viral threats.

### REFERENCES

Adler-Agnon et al., Novel promoters for constitutive and inducible expression of transgenes in the diatom phaeodactylum tricornutum under varied nitrate availability. J. Appl. Phycol. 30, 2763-2772 (2018).
Anders et al., Htseq-a python framework to work with high-throughput sequencing data. Bioinformatics 31, 166-169 (2015).
Berndt et al. Recombinant production of a functional SARS-CoV-2 spike receptor binding domain in the green algae Chlamydomonas reinhardtii. bioRxiv DOI: 10.1101/2021.01.29.428890 (2021).
Chu et al., Rapid induction of gfp expression by the nitrate reductase promoter in the diatom phaeodactylum tricornutum. PeerJ 4, e2344 (2016).
Dell’Aquila, G. et al. Mobilization and cellular distribution of phosphate in the diatom Phaeodactylum 620 tricornutum. Front. Plant Sci. 11, 579 (2020).
Erdene-Ochir et al., Identification and characterisation of the novel endogenous promoter HASP1 and its signal peptide from Phaeodactylum tricornutum. Sci. Reports 9, 1-10 (2019).
Gibson, D. G. Synthesis of DNA fragments in yeast by one-step assembly of overlapping oligonucleotides. Nucleic Acids Res. 37, 6984-6990, DOI: 10.1093/nar/gkp687 (2009).
Giguere et al. Telomere-to-telomere genome assembly of phaeodactylum tricornutum. bioRxiv DOI: 10.1101/2021.05.04.442596 (2021)
Grant et al., Analysis of the SARS-CoV-2 spike protein glycan shield reveals implications for immune recognition. Scientific Reports 10: 14991 (2020). https://doi.org/10.1038/s41598-020-71748-7
Kapust et al., The P1′ specificity of tobacco etch virus protease. Biochemical and Biophysical Research Communications. 294 (5): 949-955. (2002).
Kostallas et al., “Substrate profiling of tobacco etch virus protease using a novel fluorescence-assisted whole-cell assay”. PLOS ONE. 6 (1): e16136 (2011).
Karas et al., Designer diatom episomes delivered by bacterial conjugation. Nat. Commun. 6, 1-10 (2015).
Kim et al., Graph-based genome alignment and genotyping with hisat2 and hisat-genotype. Nat. Biotechnol. 37, 907-915 (2019).
Li, H. Minimap and miniasm: fast mapping and de novo assembly for noisy long sequences. Bioinformatics 32, 2103-2110 (2016).
Lin et al. Alkaline phosphatase promoter as an efficient driving element for exogenic recombinant in the marine diatom phaeodactylum tricornutum. Algal research 23, 58-65 (2017).
Lykkemark et al., Degradation of C-terminal tag sequences on domain antibodies purified from E. coli supernatant. In MAbs, vol. 6, 1551-1559 (Taylor & Francis, 2014).
Niu et al. Transformation of diatom phaeodactylum tricornutum by electroporation and establishment of inducible selection marker. Biotechniques 52, 1-3 (2012).
Noskov et al. Assembly of large, high G+C bacterial DNA fragments in yeast. ACS Synth. Biol. 1, 267-273, DOI: 10.1021/sb3000194 (2012).
Slattery et al. An Expanded Plasmid-Based Genetic Toolbox Enables Cas9 Genome Editing and Stable Maintenance of Synthetic Pathways in Phaeodactylum tricornutum. ACS Synth. Biol. 7, 328-338, DOI: 10.1021/acssynbio.7b00191 (2018).
Slattery et al., “Plasmid-based complementation of large deletions in Phaeodactylum tricornutum biosynthetic genes generated by Cas9 editing”. Scientific Reports 10(1): 13879 (2020). doi: 10.1038/s41598-020-70769-6.
Tian et al., O-glycosylation pattern of the sars-cov-2 spike protein reveals an “o-follow-n” rule. Cell Res. 1-3 (2021).
Walls et al., Glycan shield and epitope masking of a coronavirus spike protein observed by cryo-electron microscopy. Nature Structural & Molecular Biology. 23(10): 899-905 (2016). doi: 10.1038/nsmb.3293.
Whitman et al. Evaluation of sars-cov-2 serology assays reveals a range of test performance. Nat. biotechnology 38, 1174-1183 (2020).
Wick et al., Benchmarking of long-read assemblers for prokaryote whole genome sequencing. F1000Research 8 (2019).

1. A recombinant glycoprotein or protein comprising a coronavirus polypeptide antigen having a glycosylation or other post-translational modification pattern produced by, or characteristic of, post-translational modification by Phaeodactylum tricornutum.

2. The recombinant glycoprotein or protein of claim 1, wherein the coronavirus polypeptide antigen:
(i) has an N-linked glycosylation pattern and/or phosphorylation pattern produced by, or characteristic of, post-translational modification by P. tricornutum;
(ii) is a betacoronavirus polypeptide antigen (e.g., SARS-CoV-2, SARS-CoV, or MERS-CoV);
(iii) is from a surface glycoprotein or protein (e.g., spike (S) protein, a nucleocapsid (N) protein, a membrane protein, or an envelope protein);
(iv) is or comprises a fragment of a coronavirus spike protein (e.g., a fragment comprising S1 subunit, S2 subunit, or receptor binding domain); or a fragment of a coronavirus nucleocapsid protein (e.g., an N-terminally truncated nucleocapsid protein, such as an N-terminally truncated nucleocapsid protein lacking contiguous residues 19-110, 19-111, 19-112, 19-113, 19-114, 19-115, 19-116, 19-117, 19-118, 19-119, 19-120, 19-121, 19-122, 19-123, 19-124, 19-125, 19-126, 19-127, 19-128, 19-129, 19-130, 19-131, 19-132, 19-133, 19-134, 19-135, 19-136, 19-137, 19-138, 19-139, 19-140, 19-141, 19-142, 19-143, 19-144, 19-145, 19-146, 19-147, 19-148, 19-149, 19-150, 19-151, 19-152, 19-153, 19-154, 19-155, 19-156, 19-157, 19-158, 19-159, 19-160, 19-161, 19-162, 19-163, 19-164, 19-165, 19-166, 19-167, 19-168, 19-169, 19-170, 19-171, 19-172, 19-173, 19-174, 19-175, 19-176, 19-177, 19-178, 19-179, 19-180, 19-181, 19-182, 19-183, 19-184, 19-185, 19-186, 19-187, 19-188, 19-189, 19-190, 19-191, 19-192, 19-193, 19-194, 19-195, 19-196, 19-197, 19-198, 19-199, 19-200, 19-201, 19-202, 19-203, 19-204, 19-205, 19-206, 19-207, 19-208, 19-209, 19-210, 19-211, 19-212, 19-213, 19-214, 19-215, 19-216, 19-217, 19-218, 19-219, 19-220, 19-221, 19-222, 19-223, 19-224, 19-225, 19-226, 19-227, 19-228, 19-229, 19-230, 19-231, or 19-232 of SEQ ID NO: 23);
(v) is or comprises a fragment of a coronavirus spike protein’s receptor binding domain (RBD), wherein:
(a) the recombinant glycoprotein competitively inhibits binding of a native RBD protein produced in mammalian (e.g., human) cells to a human ACE2 receptor; and/or
(b) the recombinant glycoprotein cross-reacts with antibodies (e.g., neutralizing antibodies) raised against the spike protein or an RBD-comprising fragment thereof, or
(vi) comprises an amino acid sequence at least 70%, 75%, 80%, 85%, 90%, 95%, or 99% identical to SEQ ID NO: 1, to SEQ ID NO: 23, or to residues 19-443 of SEQ ID NO: 23, optionally further comprising an N-terminal sequence comprising residues 1-18 of SEQ ID NO: 23.

3-7. (canceled)

8. The recombinant glycoprotein or protein of claim 1, wherein the recombinant glycoprotein or protein:
(i) has an N-linked glycosylation pattern comprising core fucosylation;
(ii) has an overall length of no more than 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1000 residues; and/or
(iii) lacks a functional endoplasmic reticulum retention signal, thereby enabling the formation of complex N-linked glycosylation in the Golgi apparatus of the P. tricornutum host cells.

9-11. (canceled)

12. An immunogenic composition (e.g., vaccine) comprising the recombinant glycoprotein or protein as defined in claim 1, and a suitable adjuvant.

13. A Phaeodactylum tricornutum host cell that produces and/or preferably secretes the recombinant glycoprotein or protein as defined in claim 1, wherein the host cell comprises an exogenous expression cassette encoding the recombinant glycoprotein or protein operably linked to a promoter (e.g., an HASP1 promoter).

14. A diagnostic device comprising the recombinant glycoprotein or protein as defined in claim 1 for use in detecting the presence and/or concentration of antibodies that bind to said recombinant glycoprotein or protein.

15. The diagnostic device of claim 14, which is a lateral flow test.

16. (canceled)

17. A method for triggering the production of antibodies against a coronavirus polypeptide antigen, the method comprising administering to a subject the immunogenic composition as defined in claim 12.

18. A method for detecting antibodies specific to a coronavirus polypeptide antigen in a biological sample, the method comprising: (a) contacting the biological sample with the recombinant glycoprotein or protein as defined in claim 1 ; and (b) detecting a complex formed between antibodies specific to the coronavirus polypeptide antigen and the recombinant glycoprotein or protein.

19-24. (canceled)

25. A method for producing a recombinant glycoprotein or protein, the method comprising:
(a) providing Phaeodactylum tricornutum or other suitable host cells (e.g., diatom host cells or cells of the same clade of P. tricornutum) comprising a polynucleotide encoding the recombinant glycoprotein or protein in an expression cassette under control of an HASP1 (highly abundant secreted protein 1) promoter; and
(b) culturing the host cells in a production medium for a sufficient period of time to induce expression of the recombinant glycoprotein or protein, the production medium being maintained an inorganic phosphate concentration sufficiently low such that the recombinant glycoprotein or protein is expressed at a level higher than when the host cells are cultured under corresponding conditions in a phosphate-replete medium.

26. The method of claim 25, wherein the production medium is a phosphate-reduced production medium having:
(i) an inorganic phosphate concentration sufficiently low such that the recombinant glycoprotein or protein is expressed at a level at least 1.2, 1.3, 1.4, 1.5, 1.6, 1.7, 1.8, 1.9, or 2-fold higher than when the host cells are cultured under corresponding conditions in a phosphate-replete medium;
(ii) an inorganic phosphate concentration of less than or equal to 50%, 45%, 40%, 35%, 30%, 25%, 20%, 15%, 10%, or 5% of that present in a phosphate-replete growth medium that was used to culture the host cells provided in (a);
(iii) an inorganic phosphate concentration of less than or equal to: 30, 29, 28, 27, 26, 25, 24, 23, 22, 21, 20, 19, 18, 17, 16, 15, 14, 13, 12, 11, 10, 9, 8, 7, 6, 5, 4, 3, 2.5, 2, 1.5, or 1 µM; or 2.8, 2.7, 2.6, 2.5, 2.4, 2.3, 2.2, 2.1, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1 ppm; or
(iv) any combination or (i) to (iii).

27. The method of claim 26, wherein the host cells prior to (b) are cultured in a phosphate-replete growth medium having an inorganic phosphate concentration sufficiently high to repress expression of the recombinant glycoprotein or protein as compared to when the host cells are cultured in the phosphate-reduced production medium.

28. The method of claim 25, wherein the production medium is a phosphate-reduced and iron-reduced production medium having an inorganic phosphate concentration as defined in claim 25 and having:
(i) an iron concentration sufficiently low such that the recombinant glycoprotein or protein is secreted at a level at least 1.2, 1.3, 1.4, 1.5, 1.6, 1.7, 1.8, 1.9, or 2-fold higher than when the host cells are cultured under corresponding conditions in an iron-replete medium;
(ii) an iron concentration of less than or equal to 50%, 45%, 40%, 35%, 30%, 25%, 20%, 15%, 10%, or 5% of that present in an iron-replete growth medium that was used to culture the host cells provided in (a);
(iii) an iron concentration of less than or equal to: 10, 9, 8, 7, 6, 5, 4, 3, 2.5, 2, 1.5, 1, 0.9, 0.8, 0.7, 0.6, 0.5, 0.4, 0.3, 0.25, 0.2, 0.15, or 0.1 µM;
(iv) the recombinant protein is to be secreted from the host cells; or
(v) any combination or (i) to (iv).

29. The method of claim 26, wherein the host cells prior to (b) are cultured in an iron-replete growth medium having an iron concentration sufficiently high to repress secretion of the recombinant glycoprotein or protein as compared to when the host cells are cultured in an iron-reduced production medium, or wherein the host cells prior to (b) are cultured in a phosphate-reduced and iron-reduced medium as defined in claim 27 as a growth medium.

30. The method of claim 25, wherein the host cells are cultured in the production medium for at least 1, 1.5, 2, 2.5, 3, 3.5, 4, 4.5, or 5 days.

31. The method of claim 25, wherein the host cells are engineered to comprise the expression cassette as part of their genome.

32. The method of claim 25, wherein the recombinant glycoprotein or protein is heterologous with respect to the host cells and/or with respect to the HASP1 promoter.

33-39. (canceled)

40. A method for increasing secretion and/or expression of a recombinant glycoprotein or protein being expressed in an algae microorganism, said method comprising:
a) providing a host cell algae microorganism comprising an expression cassette or vector comprising a polynucleotide encoding the recombinant glycoprotein or protein, and wherein the polynucleotide does not encode a Tobacco Etch Virus (TEV) protease cleavage site; and
b) culturing the host cells in a production medium for a sufficient period of time to induce expression and/or secretion of the recombinant glycoprotein or protein.

41. The method of claim 40, wherein the algae microorganism is Phaeodactylum tricornutum or other suitable host cells (e.g., diatom host cells or cells of the same clade of P. tricornutum).

42. The method of claim 40, wherein the polynucleotide further encodes a cleavable purification tag (e.g., glutathione-S-transferase (GST) tag, a histidine tag [e.g., 6His or 10His], or Fc tag).

43-45. (canceled)


SEQUENCE LISTING (23 sequences)

SEQ ID NO: 1 - protein, 223 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 2 - DNA, 19559 bp, synthetic construct
  source 1..19559: mol_type = other DNA
tcgagctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttaccttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatcctagtacactctatatttttttatgcctcggtaatgattttcattttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagttttacacaggagtctggacttgacgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagcaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgacctcgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgcaaggggttcgcgtcagcgggtgttggcgggtgtcggggctggcttaactatgcggcatcagagcagattgtactgagagtgcaccatatgcggtgtgaaataccacacagatgcgtaaggagaaaataccgcatcaggcgccattcgccattcagctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccggggatcctctagagtcgacctgcaggcatgcaagcttgagtattctatagtctcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgtgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagcccatcgatactagcttgattgggatatctcgctcatgtttgtcgcgtgctatgtctttttaggtactttgaacctacgttcgtacttgtataatatgatcatcgtattatcgtttttcatccgtccagcgcaaaatgcattagcagctagtcctagcgtgcggagctacctggacaggtgcatgacggatgcgtgtccttcagtgactttctaattaacagtaacttctttacttatgtttcagtttgtaagaagcgggattcgctcgtcgcttgacatctgattggactgcgtcggcacgtgaaaactacattgtgaaatctgctaaaactccgggtatctctgacacaaaacgattcggcttcgcaatttcaacattacggtcaaggctaacgtatctttctcggtcaacttcagattatgccgattaaattgtcgtagctttcaaggcgttttgagtactgcggcagttgttgaacctgcaaggagaagatctcgacaacagaataaagcgaaaaatgggtctcatgcactaacactcaggcctccctcataatctctgtttgagtttaccaacaacacatatatacatttcgacaaaatgaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctgaccgacgccgaccaacaccgccggtccgacgcggcccgacgggtccgaggcctcggagatctgggcccatgcggccgcaacaactacctcgactttggctgggacactttcagtgaggacaagaagcttcagaagcgtgctatcgaactcaaccagggacgtgcggcacaaatgggcatccttgctctcatggtgcacgaacagttgggagtctctatccttccttaaaaatttaattttcattagttgcagtcactccgctttggtttctcgcgttttacgcgccgtcagtttcttggcccacgaactggattcccacttgttcatggtgtcggcttccgtccaggccttgagcagactcttttgacgagcgtttcgctgaatatcgaccttcaggtcggtcagcgcgaggcgtttgtaggaaataacttgtcgggtgactcccgtgatagattctgggccgtcaatcagacacttgttctggtcgacaatgtcaataatggtggcgagcttgccagcctccggtccgtagttgatcaaaaccacgcgacccaattcaacgtatcggtgaaacggcatggttgttaaagagattgaaaatgagagagaagtggtatgtgtatgtaaatgtgtgggtgatcctacgcaaaaacgagtttcgaagtagcagcgacacacgagatggagtgtagtttgttgttggtcaaaggacacggcgaggggcccaccacgagttgtccgtagtccgtttttgtcgggtaatttcgcatgctagagacggagcacggggttccgcgagcggcggatacccgcgtgcggtgatgcgggccgagaattttagtaaaccggtaatggagggttgaatagggtagggcgggaaaagaccaccaacctaccccggaaacgataggacatgtcgtagcggacatttaaaagaagagaatttcgttggatatacctacttcttggatttacatcacacaaccagtagaagataccgggtagagtaggtactcttcttcgacaccacacaaaacaccaaccaatacactctggaaaagacacttctaacgtgaaaggagatcatcgtccccacaatttgccgagtgatattgttcgcttcgcaacaacctagcagactctgctggtgtcacattttcggacacattttcgtctgccaaggccgcgtacacacacgcacatattcataaaactcgttcggaaatcgtttcatgtcgagttttccgtatcccgcctttctccaccaccgtccggtggacgccgcggcgcagcgtgccgcactcgcggccgttggtcgttgccgcatcgtccctacatcgtcctttcctggggccgctgactttcgcgagattgtccaaatgaacgtggcacaagcacacgtcggactcacggacgagaccgacggtgcggatcaggaaacgctccagaccacacgcaaggcccggatgaaacagggcatcgccatggccaagacgtatctcgtcgaaaccttacagttggatgtcacggtcgtggatgtggaagtcgttttggatgctcgggatgtgattcaatcggacggctgtctcgcgagtggactcctcgatgcgggttgtgacaaaattgtcgtggacggcaccaatcgggacgcttttgagactgcacaaattccgaaagcgagactgatgggccactttgtgtatccccataccgattcacgaacccgaaacgggagtctcgccgtggcctttgccgaagtccaacccttttgcgattccgtcagtgtcgagctatcgaatcctgaagccgtcacggaaagtgtcatccaggaaattgtgcaagcggcaaaagcccagcaattgcaaatttcaatcgtctacaatcccaacgactacgagaatcctggtgatcacgccgccatggttggtcgcgtatgccagagcttcccagaagggcaagtccggttggccttgaccgatccgaccgcctcgcaactcggacacgcctacgcggcgtgtctcaaaaccgaccgggacgacggtctctttaccaccgtcgtctgtactcgcaacaatgaagcactggggcttgtctactcctccacgtcgtctattgtcgccgcactggaatgtggtcggggtgtctactacagccgttcccggaacggattgtggcgcaagggcgacacgtccggacactttcaggtacttcaccggctcgacaaagactgtgacggggatgctttgcgggtgacggtcactcaaaaaggcacccctgccgcattttgccacttgaatactctaacgtgctggggtacgtcgagaggcttgcgacatttggaagaaacgctgcaagaccgactcgtgtcggctccggaaggatcctacacgaaacgtctgtttcaagacgagacactgttgcgggacaagctggtcgaagaagcgcaggaattgtcggaagccgatacgaaggcacacgtggcggaagaactggcggatgtcctctactttgccatggtgagggcggtcaaggctggagtcagtttggatgatgccgtcgcagagctagattctagggcgcgaaaagtgacgcgacgaaaaggcgatagtaaagcctttcgaattgcagcgggtgaagctattttgaaaaaagcgcagtcgtagcaacgcaagaaatagattcgtatacggaatctaggatacctccttggatattaatagcacatttctgtccttggtagaatcttgagtgttgcagcggaacgataaagggtcgaatggtcaggctgagctgaatagtccccactgagttctatccggaacggacttgcgacgaggagctgccaaagtgcaggcgtgcttccattgagtcccaacctatgctgctagcggtgtgttcgcttttagaccttgaccggggtccagacttgtctaccgaagcagggtactccgactcatccactagatgaagcggcgcatctagaaatgctccattgggaaacaaatcgtccaagctatcaaaactggatgagacactggccaacgaagtcatctcggaaatcccatcattgtattcgactgaccctccgactagccagttgcgtttgggaatttccatgtgatttggaaggcgaatcaagcgtgggaccaagaatcgatgaggagaaagcggagggagacgcctgtcatctttatgtaatcgcgggggattggtgactttgtcagcgttgatgttaccgtctgcggcgatcaaaagtgtcgcttggtcaatgtcgcttgtcaaggtcgaggtgtacgaaagttctggactatcagcaccactctgttgggcgcctcgagactggacaaagtcctctgaatttttcaaaggtaaaccaagctgtaataagttgttgcgaacggcggcctgcaagcggccgtacatagcggtcccagggagagggcttggtcgagtgggaacggcatctcgctgctgctctgtcttgttcattgtcccagatgttgccgtttgagtaaagggatgcttaattttaccacatgctgtgtctttgagctgaacaggggtgtgtttgaatgattccatgtcgctgccaccatgacatgagcaactctcgcggggctttgtagtgctccctttcccagctactcgacgcatcaggcgggcggattgatagtgaaaaccttattcattgtcagagcttaagccggtctggtctatctttccactgtcaaacagctcttgattgtcgcccgcgcgaaaatagtagcactaactgtaacttcaaaatacaaaatgttctctgttaccatacagtgaatgtaactttcgaattgacagtattagtagtcgtattgacagtgaggcacgcccctcaatgtgcgaggtggaaaatataccagcatgacaatgaatcttggagattcttttgctgtcatcaagattcaccgccaaatcttcaggaacctatcacgtccacaggcgatgttaattcttgagtcgtcaaaacaaagtcctgtcctacctgtagaagttgacagcgagcaattgtatgcaaacttctgactttgttataataacattaaaggtaattaagtatcttcaattaggcattttgtcactgtcagtccgttccgacaatataggtagatttggaatgaatcttttctatgctgctgcgaatcttgtacacctttgaggccgtagattctgtccgacgaagcgataattattgcaaaatacatggactcattattttgattcgatttctttttggtatccgactcgaaaagatccatcacggcgagcatgaatcttcgttgtatccttccgtttctcctcgcaagcttctcggctggggctcgtgtgcagcccactgagtccatcgtgcgcttcccgaatattacgaatctttgcccctttggagaagtcttcaatgccactcgtttcgcctccgtttacgcctggaaccgtaagcgcatctccaattgcgtcgccgattactccgttctttacaactcggcttcgttttcgactttcaaatgctacggtgtgtccccgactaagctcaacgacctctgttttacgaatgtgtacgctgattcctttgtcattcgtggtgacgaagtccgccagatcgctccgggtcagaccggaaagattgcggactacaactacaaactgccggacgacttcactggatgcgtgattgcctggaattccaataacttggattcgaaagttggaggtaattacaattacttgtaccgtcttttccgcaagtcgaaccttaaaccctttgaacgtgacatttccaccgaaatttaccaagccggctcgaccccctgtaacggagtggaaggctttaattgctacttcccccttcaatcgtacggattccagccgactaacggtgttggataccaaccctaccgtgtggtggtcctgtccttcgaactgctccacgctccggcgactgtctgtggacccaagaaatcgaccaatctggtcaagaataaatgtgtcaattttgaaaacctctacttccagggacaccatcaccatcaccattgaagataagaatatctcattgtgaacatctatgatttaccaattttattctttgtttacagttagacgccagtaattgtgctgtttctctcaagtctgtgtcaatacaaactacgaaacttggcaatttttctcttgaatatgagcacgagattgaaacgcacaaaggaaattagtttccatcctttgacaaagtttgttgctgtttagagaacagatgtcaaaattaacgtgccatggaattgaaccatggtgcgctatccccaaatcacgtcgtttgacctcgtcacatttaaggtatatcaagcatattcacttatatcttgacatcctccctgcttgatattcctttgcccttgagccatcttccccacacgctggagatgacccctgcccctcctttctttatccacccgagtaaggtgtcgacaagtcactttgcccctgagccatcctccccactcaggagatgacccctacccctttcttctttatccaccccatcttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagaggcacttcgagctgtaagtacatcaccgacgagcaaggcaagacgatccgcgcctgttttattgagaacgttgttcgtgttggcctcaatggtagcgatgcgtcattcagcgaagttctggtgctgatgatgtggttcgctttgccactggtcaatgtggtaagcccgtgtaatgtcagtaacctttttactgatctcagcttgagcacggtcgctgatgagcttatccatggccccacggtaacggatatgatcctctagggcgttgacaaattctttgtcggttttcatggggtagacgtcagtgaccagggaacgtctccctacaaaaagttgcgcatactttgctccgttgtcgacggcaggggtgtcggaccagattgtatctgtggcaacggcctcattgcgtcaatggacctttaaagccgggaaacgagacttgaaatgtttacgtagaggtgcattatagacctcacgcgcatattgagtggttgcaaaaatggttttgcgaacggtatcactggagacccatgccaggcaaggacggagggcatcataatcatgttcattgcgttggacagcatgcttgttacacgtaagtatacggtcgagagcatcgtgacgttgagaattacagatggcaacgtgtcggtgcatgtattggccgagattgtcaaatcgtggctcaatatatggcatgccaggtaagtcatggatgtcggtatgccattcagccttcatgtcgatttcgttgtcgagaactgatgggtcccagtcaacgtcagaagtaagaacaacatggggaagggaatgaagttcatgactggtgggtgcgcgcatatccatgtacggaagaccctgtcgtatgtttagtgggatgatgtaatcatccagggtaacaatacgttggttgccgcccacggtacgagaccgatcttgaacacaattgtggcaatgttcaagttgggcgctggaatggatggttttacctttaccaagatgcgcgtattgatgcatgataacaacaatgggccctcgttgcgattcgacaagaccggcagccgtgacaatgtctagattggacaaggtatgttcatttataccggttatgttggctgaacgtccagttttgtgcagtacggtaacatcactccccgcaagtccaccgttggcaccgcgatcgacaagtgcagatgtggtattttgtactgtgtgtcgggagacttggtactgtaacacatttgactgaagtgacttggtggtaccatgaggacggagggaacccgttgatacggtgttgcgtgacgctgcaagtaccttacgtatgtcgcctggatccatacgtccaacccgatcagaaagatgtgctaggagttccgtttctggtgcacaatcatgaaaggtatccacgggagttccattgttgtcgtccggggctgcgtcgcgactgccagttgcatgtacatgcgccatcaagggacgtgccaatccattactaaccgggagaaccttagctggggcagccaatccctgaagtatagccttagcatcatctgagagctggttccacatatctctagggatataaggtcgttcacggttagtgggcgtactatgatgtcggttattagtacaattctccctgcgggcatgcgcattggcttcatagagtacggaaggtgacaagtcaatattgtagttaacatccggatcagtgtcaaagtctgtaggatggtaagaaagatcagtagaatgaatactacgcttttccttgggactacgggaattagagaagttgtttcctttattgtagagtgacgctgaagcaagtagaagactaaggtaactctcgtagctaataggattacctcctttggctaggtcaagagtggctgtgatcttcacttgacaaagttccggtacattgtggacagcattctccaaaagactaagacacagttgctttgggagttgctcagccattggtacagtattgtggtagatacaaaggtggttcttccaatgaaggataaatccttctgctgtgcctgtccatgaggatccatatttcgccgtagttaggtaaccaagcgtggtggctgaactgatcttcgcacttgctgattccgtatagtgtttgacaactttacaaaacacttcttgcgcagttcgctctagcctggacagagaagggacaatcttcgtcgttaagactcgtgcgaatagcaaaagatcacaaaatagcacatcggcaccgaccaacgattatttccaaggaaaaaaagaatgcttcactacaagaaattgtgtcatccctatacagagtcttgttactgtgacagaaaattgatggaagatgtggcggattgcctttacactagccaacttgttcgactaattgcagcttcttctgagaggcttcaccgagtaacgcgaagaacaccggtgtctcgtacatgctcgtcggtgaacgctcgtccaatgacaccccccactttgtatcaatatcccaacttggtagtgaactggaatgatacatgcaatttcgcgccgcatcaacagccacgggccaccatcgacgaatagactcgg

SEQ ID NO: 3 - DNA, 19555 bp, synthetic construct
  source 1..19555: mol_type = other DNA
tcgagctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttaccttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatcctagtacactctatatttttttatgcctcggtaatgattttcattttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagttttacacaggagtctggacttgacgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagcaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgacctcgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgcaaggggttcgcgtcagcgggtgttggcgggtgtcggggctggcttaactatgcggcatcagagcagattgtactgagagtgcaccatatgcggtgtgaaataccacacagatgcgtaaggagaaaataccgcatcaggcgccattcgccattcagctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccggggatcctctagagtcgacctgcaggcatgcaagcttgagtattctatagtctcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgtgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagcccatcgatactagcttgattgggatatctcgctcatgtttgtcgcgtgctatgtctttttaggtactttgaacctacgttcgtacttgtataatatgatcatcgtattatcgtttttcatccgtccagcgcaaaatgcattagcagctagtcctagcgtgcggagctacctggacaggtgcatgacggatgcgtgtccttcagtgactttctaattaacagtaacttctttacttatgtttcagtttgtaagaagcgggattcgctcgtcgcttgacatctgattggactgcgtcggcacgtgaaaactacattgtgaaatctgctaaaactccgggtatctctgacacaaaacgattcggcttcgcaatttcaacattacggtcaaggctaacgtatctttctcggtcaacttcagattatgccgattaaattgtcgtagctttcaaggcgttttgagtactgcggcagttgttgaacctgcaaggagaagatctcgacaacagaataaagcgaaaaatgggtctcatgcactaacactcaggcctccctcataatctctgtttgagtttaccaacaacacatatatacatttcgacaaaatgaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccgactgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctgaccgacgccgaccaacaccgccggtccgacgcggcccgacgggtccgaggcctcggagatctgggcccatgcggccgcaacaactacctcgactttggctgggacactttcagtgaggacaagaagcttcagaagcgtgctatcgaactcaaccagggacgtgcggcacaaatgggcatccttgctctcatggtgcacgaacagttgggagtctctatccttccttaaaaatttaattttcattagttgcagtcactccgctttggtttctcgcgttttacgcgccgtcagtttcttggcccacgaactggattcccacttgttcatggtgtcggcttccgtccaggccttgagcagactcttttgacgagcgtttcgctgaatatcgaccttcaggtcggtcagcgcgaggcgtttgtaggaaataacttgtcgggtgactcccgtgatagattctgggccgtcaatcagacacttgttctggtcgacaatgtcaataatggtggcgagcttgccagcctccggtccgtagttgatcaaaaccacgcgacccaattcaacgtatcggtgaaacggcatggttgttaaagagattgaaaatgagagagaagtggtatgtgtatgtaaatgtgtgggtgatcctacgcaaaaacgagtttcgaagtagcagcgacacacgagatggagtgtagtttgttgttggtcaaaggacacggcgaggggcccaccacgagttgtccgtagtccgtttttgtcgggtaatttcgcatgctagagacggagcacggggttccgcgagcggcggatacccgcgtgcggtgatgcgggccgagaattttagtaaaccggtaatggagggttgaatagggtagggcgggaaaagaccaccaacctaccccggaaacgataggacatgtcgtagcggacatttaaaagaagagaatttcgttggatatacctacttcttggatttacatcacacaaccagtagaagataccgggtagagtaggtactcttcttcgacaccacacaaaacaccaaccaatacactctggaaaagacacttctaacgtgaaaggagatcatcgtccccacaatttgccgagtgatattgttcgcttcgcaacaacctagcagactctgctggtgtcacattttcggacacattttcgtctgccaaggccgcgtacacacacgcacatattcataaaactcgttcggaaatcgtttcatgtcgagttttccgtatcccgcctttctccaccaccgtccggtggacgccgcggcgcagcgtgccgcactcgcggccgttggtcgttgccgcatcgtccctacatcgtcctttcctggggccgctgactttcgcgagattgtccaaatgaacgtggcacaagcacacgtcggactcacggacgagaccgacggtgcggatcaggaaacgctccagaccacacgcaaggcccggatgaaacagggcatcgccatggccaagacgtatctcgtcgaaaccttacagttggatgtcacggtcgtggatgtggaagtcgttttggatgctcgggatgtgattcaatcggacggctgtctcgcgagtggactcctcgatgcgggttgtgacaaaattgtcgtggacggcaccaatcgggacgcttttgagactgcacaaattccgaaagcgagactgatgggccactttgtgtatccccataccgattcacgaacccgaaacgggagtctcgccgtggcctttgccgaagtccaacccttttgcgattccgtcagtgtcgagctatcgaatcctgaagccgtcacggaaagtgtcatccaggaaattgtgcaagcggcaaaagcccagcaattgcaaatttcaatcgtctacaatcccaacgactacgagaatcctggtgatcacgccgccatggttggtcgcgtatgccagagcttcccagaagggcaagtccggttggccttgaccgatccgaccgcctcgcaactcggacacgcctacgcggcgtgtctcaaaaccgaccgggacgacggtctctttaccaccgtcgtctgtactcgcaacaatgaagcactggggcttgtctactcctccacgtcgtctattgtcgccgcactggaatgtggtcggggtgtctactacagccgttcccggaacggattgtggcgcaagggcgacacgtccggacactttcaggtacttcaccggctcgacaaagactgtgacggggatgctttgcgggtgacggtcactcaaaaaggcacccctgccgcattttgccacttgaatactctaacgtgctggggtacgtcgagaggcttgcgacatttggaagaaacgctgcaagaccgactcgtgtcggctccggaaggatcctacacgaaacgtctgtttcaagacgagacactgttgcgggacaagctggtcgaagaagcgcaggaattgtcggaagccgatacgaaggcacacgtggcggaagaactggcggatgtcctctactttgccatggtgagggcggtcaaggctggagtcagtttggatgatgccgtcgcagagctagattctagggcgcgaaaagtgacgcgacgaaaaggcgatagtaaagcctttcgaattgcagcgggtgaagctattttgaaaaaagcgcagtcgtagcaacgcaagaaatagattcgtatacggaatctaggatacctccttggatattaatagcacatttctgtccttggtagaatcttgagtgttgcagcggaacgataaagggtcgaatggtcaggctgagctgaatagtccccactgagttctatccggaacggacttgcgacgaggagctgccaaagtgcaggcgtgcttccattgagtcccaacctatgctgctagcggtgtgttcgcttttagaccttgaccggggtccagacttgtctaccgaagcagggtactccgactcatccactagatgaagcggcgcatctagaaatgctccattgggaaacaaatcgtccaagctatcaaaactggatgagacactggccaacgaagtcatctcggaaatcccatcattgtattcgactgaccctccgactagccagttgcgtttgggaatttccatgtgatttggaaggcgaatcaagcgtgggaccaagaatcgatgaggagaaagcggagggagacgcctgtcatctttatgtaatcgcgggggattggtgactttgtcagcgttgatgttaccgtctgcggcgatcaaaagtgtcgcttggtcaatgtcgcttgtcaaggtcgaggtgtacgaaagttctggactatcagcaccactctgttgggcgcctcgagactggacaaagtcctctgaatttttcaaaggtaaaccaagctgtaataagttgttgcgaacggcggcctgcaagcggccgtacatagcggtcccagggagagggcttggtcgagtgggaacggcatctcgctgctgctctgtcttgttcattgtcccagatgttgccgtttgagtaaagggatgcttaattttaccacatgctgtgtctttgagctgaacaggggtgtgtttgaatgattccatgtcgctgccaccatgacatgagcaactctcgcggggctttgtagtgctccctttcccagctactcgacgcatcaggcgggcggattgatagtgaaaaccttattcattgtcagagcttaagccggtctatctttccactgtcaaacagctcttgattgtcgcccgcgcgaaaatagtagcactaactgtaacttcaaaatacaaaatgttctctgttaccatacagtgaatgtaactttcgaattgacagtattagtagtcgtattgacagtgaggcacgcccctcaatgtgcgaggtggaaaatataccagcatgacaatgaatcttggagattcttttgctgtcatcaagattcaccgccaaatcttcaggaacctatcacgtccacaggcgatgttaattcttgagtcgtcaaaacaaagtcctgtcctacctgtagaagttgacagcgagcaattgtatgcaaacttctgacttatgttataataacattaaaggtaattaagtatcttcaatttggcattttgtcactgtcagtccgttccgacaatacaggtagatttggaatgaatcttttctatgctgctgcgaatcttgtacaccttcgaggccgtagattctgtccgacgaagcgataattattgcaaaatacatggactcattattttgattcgatttctttttggtatccgactcgaaaagatccatcacggcgagcatgaatcttcgttgtatccttccgtttctcctcgcaagcttctcggctggggctcgtgtgcagcccactgagtccatcgtgcgcttcccgaatattacgaatctttgcccctttggagaagtcttcaatgccactcgtttcgcctccgtttacgcctggaaccgtaagcgcatctccaattgcgtcgccgattactccgttctttacaactcggcttcgttttcgactttcaaatgctacggtgtgtccccgactaagctcaacgacctctgttttacgaatgtgtacgctgattcctttgtcattcgtggtgacgaagtccgccagatcgctccgggtcagaccggaaagattgcggactacaactacaaactgccggacgacttcactggatgcgtgattgcctggaattccaataacttggattcgaaagttggaggtaattacaattacttgtaccgtcttttccgcaagtcgaaccttaaaccctttgaacgtgacatttccaccgaaatttaccaagccggctcgaccccctgtaacggagtggaaggctttaattgctacttcccccttcaatcgtacggattccagccgactaacggtgttggataccaaccctaccgtgtggtggtcctgtccttcgaactgctccacgctccggcgactgtctgtggacccaagaaatcgaccaatctggtcaagaataaatgtgtcaattttgaaaacctctacttccagggacaccatcaccatcaccattgaagataagaatatctcattgtgaacatctatgatttaccaattttattctttgtttacagttagacgccagtaattgtgctgtttctctcaagtctgtgtcaatacaaactacgaaacttggcaatttttctcttgaatatgagcacgagattgaaacgcacaaaggaaattagtttccatcctttgacaaagtttgttgctgtttagagaacagatgtcaaaattaacgtgccatggaattgaaccatggtgcgctatccccaaatcacgtcgtttgacctcgtcacatttaaggtatatcaagcatattcacttatatcttgacatcctccctgcttgatattcctttgcccttgagccatcttccccacacgctggagatgacccctgcccctcctttctttatccacccgagtaaggtgtcgacaagtcactttgcccctgagccatcctccccactcaggagatgacccctacccctttcttctttatccaccccatcttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagaggcacttcgagctgtaagtacatcaccgacgagcaaggcaagacgatccgcgcctgttttattgagaacgttgttcgtgttggcctcaatggtagcgatgcgtcattcagcgaagttctggtgctgatgatgtggttcgctttgccactggtcaatgtggtaagcccgtgtaatgtcagtaacctttttactgatctcagcttgagcacggtcgctgatgagcttatccatggccccacggtaacggatatgatcctctagggcgttgacaaattctttgtcggttttcatggggtagacgtcagtgaccagggaacgtctccctacaaaaagttgcgcatactttgctccgttgtcgacggcaggggtgtcggaccagattgtatctgtggcaacggcctcattgcgtcaatggacctttaaagccgggaaacgagacttgaaatgtttacgtagaggtgcattatagacctcacgcgcatattgagtggttgcaaaaatggttttgcgaacggtatcactggagacccatgccaggcaaggacggagggcatcataatcatgttcattgcgttggacagcatgcttgttacacgtaagtatacggtcgagagcatcgtgacgttgagaattacagatggcaacgtgtcggtgcatgtattggccgagattgtcaaatcgtggctcaatatatggcatgccaggtaagtcatggatgtcggtatgccattcagccttcatgtcgatttcgttgtcgagaactgatgggtcccagtcaacgtcagaagtaagaacaacatggggaagggaatgaagttcatgactggtgggtgcgcgcatatccatgtacggaagaccctgtcgtatgtttagtgggatgatgtaatcatccagggtaacaatacgttggttgccgcccacggtacgagaccgatcttgaacacaattgtggcaatgttcaagttgggcgctggaatggatggttttacctttaccaagatgcgcgtattgatgcatgataacaacaatgggccctcgttgcgattcgacaagaccggcagccgtgacaatgtctagattggacaaggtatgttcatttataccggttatgttggctgaacgtccagttttgtgcagtacggtaacatcactccccgcaagtccaccgttggcaccgcgatcgacaagtgcagatgtggtattttgtactgtgtgtcgggagacttggtactgtaacacatttgactgaagtgacttggtggtaccatgaggacggagggaacccgttgatacggtgttgcgtgacgctgcaagtaccttacgtatgtcgcctggatccatacgtccaacccgatcagaaagatgtgctaggagttccgtttctggtgcacaatcatgaaaggtatccacgggagttccattgttgtcgtccggggctgcgtcgcgactgccagttgcatgtacatgcgccatcaagggacgtgccaatccattactaaccgggagaaccttagctggggcagccaatccctgaagtatagccttagcatcatctgagagctggttccacatatctctagggatataaggtcgttcacggttagtgggcgtactatgatgtcggttattagtacaattctccctgcgggcatgcgcattggcttcatagagtacggaaggtgacaagtcaatattgtagttaacatccggatcagtgtcaaagtctgtaggatggtaagaaagatcagtagaatgaatactacgcttttccttgggactacgggaattagagaagttgtttcctttattgtagagtgacgctgaagcaagtagaagactaaggtaactctcgtagctaataggattacctcctttggctaggtcaagagtggctgtgatcttcacttgacaaagttccggtacattgtggacagcattctccaaaagactaagacacagttgctttgggagttgctcagccattggtacagtattgtggtagatacaaaggtggttcttccaatgaaggataaatccttctgctgtgcctgtccatgaggatccatatttcgccgtagttaggtaaccaagcgtggtggctgaactgatcttcgcacttgctgattccgtatagtgtttgacaactttacaaaacacttcttgcgcagttcgctctagcctggacagagaagggacaatcttcgtcgttaagactcgtgcgaatagcaaaagatcacaaaatagcacatcggcaccgaccaacgattatttccaaggaaaaaaagaatgcttcactacaagaaattgtgtcatccctatacagagtcttgttactgtgacagaaaattgatggaagatgtggcggattgcctttacactagccaacttgttcgactaattgcagcttcttctgagaggcttcaccgagtaacgcgaagaacaccggtgtctcgtacatgctcgtcggtgaacgctcgtccaatgacaccccccactttgtatcaatatcccaacttggtagtgaactggaatgatacatgcaatttcgcgccgcatcaacagccacgggccaccatcgacgaatagactcgg

SEQ ID NO: 4 - DNA, 22667 bp, synthetic construct
  source 1..22667: mol_type = other DNA
tcgagctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttaccttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatcctagtacactctatatttttttatgcctcggtaatgattttcattttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagttttacacaggagtctggacttgacgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagcaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgacctcgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgcaaggggttcgcgtcagcgggtgttggcgggtgtcggggctggcttaactatgcggcatcagagcagattgtactgagagtgcaccatatgcggtgtgaaataccacacagatgcgtaaggagaaaataccgcatcaggcgccattcgccattcagctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccggggatcctctagagtcgacctgcaggcatgcaagcttgagtattctatagtctcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgtgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagcccatcgatactagcttgattgggatatctcgctcatgtttgtcgcgtgctatgtctttttaggtactttgaacctacgttcgtacttgtataatatgatcatcgtattatcgtttttcatccgtccagcgcaaaatgcattagcagctagtcctagcgtgcggagctacctggacaggtgcatgacggatgcgtgtccttcagtgactttctaattaacagtaacttctttacttatgtttcagtttgtaagaagcgggattcgctcgtcgcttgacatctgattggactgcgtcggcacgtgaaaactacattgtgaaatctgctaaaactccgggtatctctgacacaaaacgattcggcttcgcaatttcaacattacggtcaaggctaacgtatctttctcggtcaacttcagattatgccgattaaattgtcgtagctttcaaggcgttttgagtactgcggcagttgttgaacctgcaaggagaagatctcgacaacagaataaagcgaaaaatgggtctcatgcactaacactcaggcctccctcataatctctgtttgagtttaccaacaacacatatatacatttcgacaaaatgaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctgaccgacgccgaccaacaccgccggtccgacgcggcccgacgggtccgaggcctcggagatctgggcccatgcggccgcaacaactacctcgactttggctgggacactttcagtgaggacaagaagcttcagaagcgtgctatcgaactcaaccagggacgtgcggcacaaatgggcatccttgctctcatggtgcacgaacagttgggagtctctatccttccttaaaaatttaattttcattagttgcagtcactccgctttggtttctcgcgttttacgcgccgtcagtttcttggcccacgaactggattcccacttgttcatggtgtcggcttccgtccaggccttgagcagactcttttgacgagcgtttcgctgaatatcgaccttcaggtcggtcagcgcgaggcgtttgtaggaaataacttgtcgggtgactcccgtgatagattctgggccgtcaatcagacacttgttctggtcgacaatgtcaataatggtggcgagcttgccagcctccggtccgtagttgatcaaaaccacgcgacccaattcaacgtatcggtgaaacggcatggttgttaaagagattgaaaatgagagagaagtggtatgtgtatgtaaatgtgtgggtgatcctacgcaaaaacgagtttcgaagtagcagcgacacacgagatggagtgtagtttgttgttggtcaaaggacacggcgaggggcccaccacgagttgtccgtagtccgtttttgtcgggtaatttcgcatgctagagacggagcacggggttccgcgagcggcggatacccgcgtgcggtgatgcgggccgagaattttagtaaaccggtaatggagggttgaatagggtagggcgggaaaagaccaccaacctaccccggaaacgataggacatgtcgtagcggacatttaaaagaagagaatttcgttggatatacctacttcttggatttacatcacacaaccagtagaagataccgggtagagtaggtactcttcttcgacaccacacaaaacaccaaccaatacactctggaaaagacacttctaacgtgaaaggagatcatcgtccccacaatttgccgagtgatattgttcgcttcgcaacaacctagcagactctgctggtgtcacattttcggacacattttcgtctgccaaggccgcgtacacacacgcacatattcataaaactcgttcggaaatcgtttcatgtcgagttttccgtatcccgcctttctccaccaccgtccggtggacgccgcggcgcagcgtgccgcactcgcggccgttggtcgttgccgcatcgtccctacatcgtcctttcctggggccgctgactttcgcgagattgtccaaatgaacgtggcacaagcacacgtcggactcacggacgagaccgacggtgcggatcaggaaacgctccagaccacacgcaaggcccggatgaaacagggcatcgccatggccaagacgtatctcgtcgaaaccttacagttggatgtcacggtcgtggatgtggaagtcgttttggatgctcgggatgtgattcaatcggacggctgtctcgcgagtggactcctcgatgcgggttgtgacaaaattgtcgtggacggcaccaatcgggacgcttttgagactgcacaaattccgaaagcgagactgatgggccactttgtgtatccccataccgattcacgaacccgaaacgggagtctcgccgtggcctttgccgaagtccaacccttttgcgattccgtcagtgtcgagctatcgaatcctgaagccgtcacggaaagtgtcatccaggaaattgtgcaagcggcaaaagcccagcaattgcaaatttcaatcgtctacaatcccaacgactacgagaatcctggtgatcacgccgccatggttggtcgcgtatgccagagcttcccagaagggcaagtccggttggccttgaccgatccgaccgcctcgcaactcggacacgcctacgcggcgtgtctcaaaaccgaccgggacgacggtctctttaccaccgtcgtctgtactcgcaacaatgaagcactggggcttgtctactcctccacgtcgtctattgtcgccgcactggaatgtggtcggggtgtctactacagccgttcccggaacggattgtggcgcaagggcgacacgtccggacactttcaggtacttcaccggctcgacaaagactgtgacggggatgctttgcgggtgacggtcactcaaaaaggcacccctgccgcattttgccacttgaatactctaacgtgctggggtacgtcgagaggcttgcgacatttggaagaaacgctgcaagaccgactcgtgtcggctccggaaggatcctacacgaaacgtctgtttcaagacgagacactgttgcgggacaagctggtcgaagaagcgcaggaattgtcggaagccgatacgaaggcacacgtggcggaagaactggcggatgtcctctactttgccatggtgagggcggtcaaggctggagtcagtttggatgatgccgtcgcagagctagattctagggcgcgaaaagtgacgcgacgaaaaggcgatagtaaagcctttcgaattgcagcgggtgaagctattttgaaaaaagcgcagtcgtagcaacgcaagaaatagattcgtatacggaatctaggatacctccttggatattaatagcacatttctgtccttggtagaatcttgagtgttgcagcggaacgataaagggtcgaatggtcaggctgagctgaatagtccccactgagttctatccggaacggacttgcgacgaggagctgccaaagtgcaggcgtgcttccattgagtcccaacctatgctgctagcggtgtgttcgcttttagaccttgaccggggtccagacttgtctaccgaagcagggtactccgactcatccactagatgaagcggcgcatctagaaatgctccattgggaaacaaatcgtccaagctatcaaaactggatgagacactggccaacgaagtcatctcggaaatcccatcattgtattcgactgaccctccgactagccagttgcgtttgggaatttccatgtgatttggaaggcgaatcaagcgtgggaccaagaatcgatgaggagaaagcggagggagacgcctgtcatctttatgtaatcgcgggggattggtgactttgtcagcgttgatgttaccgtctgcggcgatcaaaagtgtcgcttggtcaatgtcgcttgtcaaggtcgaggtgtacgaaagttctggactatcagcaccactctgttgggcgcctcgagactggacaaagtcctctgaatttttcaaaggtaaaccaagctgtaataagttgttgcgaacggcggcctgcaagcggccgtacatagcggtcccagggagagggcttggtcgagtgggaacggcatctcgctgctgctctgtcttgttcattgtcccagatgttgccgtttgagtaaagggatgcttaattttaccacatgctgtgtctttgagctgaacaggggtgtgtttgaatgattccatgtcgctgccaccatgacatgagcaactctcgcggggctttgtagtgctccctttcccagctactcgacgcatcaggcgggcggattgatagtgaaaaccttattcattgtcagagcttaagccggtctggtctatctttccactgtcaaacagctcttgattgtcgcccgcgcgaaaatagtagcactaactgtaacttcaaaatacaaaatgttctctgttaccatacagtgaatgtaactttcgaattgacagtattagtagtcgtattgacagtgaggcacgcccctcaatgtgcgaggtggaaaatataccagcatgacaatgaatcttggagattcttttgctgtcatcaagattcaccgccaaatcttcaggaacctatcacgtccacaggcgatgttaattcttgagtcgtcaaaacaaagtcctgtcctacctgtagaagttgacagcgagcaattgtatgcaaacttctgactttgttataataacattaaaggtaattaagtatcttcaattaggcattttgtcactgtcagtccgttccgacaatataggtagatttggaatgaatcttttctatgctgctgcgaatcttgtacacctttgaggccgtagattctgtccgacgaagcgataattattgcaaaatacatggactcattattttgattcgatttctttttggtatccgactcgaaaagatccatcacggcgagcatgaatcttcgttgtatccttccgtttctcctcgcaagcttctcggctggggcttgcgtcaatctgactactcgtacccagcttccgcccgcttacaccaactctttcacccgaggcgtttactacccggacaaagttttccgttcctcggtccttcactcgacgcaggacctttttttgccgttcttttcgaacgtcacttggttccacgcgattcacgtttccggcacgaatggtacgaaacgttttgataatcccgttctgccctttaatgacggtgtctacttcgcgtccactgaaaaatcgaacatcatccgtggctggatctttggcactacgctcgattccaaaacccaatcgctgctcatcgttaataatgccaccaatgtcgttattaaagtctgcgagttccaattttgtaatgacccgtttcttggtgtgtactaccacaagaacaacaagtcgtggatggagtccgagttccgtgtgtactcctccgccaacaactgtacttttgaatacgtttcgcaaccctttcttatggatttggaaggcaaacagggcaactttaaaaatctgcgtgaatttgtcttcaagaatattgacggttacttcaaaatttactccaaacacactccgattaacttggtgcgcgatcttccccaaggtttctccgcgctcgaaccgctggtcgaccttccgatcggtatcaacattacccgcttccagactctcctggcgcttcaccgttcgtacctgactcccggtgattcgtcctcgggttggacggcgggtgcggcggcttactacgttggctaccttcaaccgcgcacgtttttgttgaagtacaacgagaacggtaccatcactgatgcggtggactgcgctttggacccgctttccgagaccaaatgtactcttaagtcctttacggtcgagaaaggtatctaccaaacttctaatttccgtgtgcagcccactgagtccatcgtgcgcttcccgaatattacgaatctttgcccctttggagaagtcttcaatgccactcgtttcgcctccgtttacgcctggaaccgtaagcgcatctccaattgcgtcgccgattactccgttctttacaactcggcttcgttttcgactttcaaatgctacggtgtgtccccgactaagctcaacgacctctgttttacgaatgtgtacgctgattcctttgtcattcgtggtgacgaagtccgccagatcgctccgggtcagaccggaaagattgcggactacaactacaaactgccggacgacttcactggatgcgtgattgcctggaattccaataacttggattcgaaagttggaggtaattacaattacttgtaccgtcttttccgcaagtcgaaccttaaaccctttgaacgtgacatttccaccgaaatttaccaagccggctcgaccccctgtaacggagtggaaggctttaattgctacttcccccttcaatcgtacggattccagccgactaacggtgttggataccaaccctaccgtgtggtggtcctgtccttcgaactgctccacgctccggcgactgtctgtggacccaagaaatcgaccaatctggtcaagaataaatgtgtcaattttaattttaacggtttgacgggaacgggtgtgctcaccgagtccaacaaaaagttccttcccttccagcagtttggtcgcgatattgcggataccacggacgcggtgcgtgatccgcaaaccctggaaattttggatattactccctgctcctttggtggtgtttccgtcattaccccgggcaccaatacttcgaatcaggttgcggtcctctaccaggacgtgaattgcactgaagtccccgtcgccattcacgccgaccagcttactccgacgtggcgcgtgtactccaccggctccaatgtttttcaaacccgtgcgggctgcctgatcggcgccgagcacgttaacaattcctacgagtgcgacatccccatcggagccggcatctgcgcttcgtaccagactcaaacgaattcgccgcgtcgcgcccgttccgtggcgtcccagtcgattattgcgtacaccatgtccctgggagcggaaaactccgttgcctactcgaataattcgattgcgatccccaccaacttcactatctcggtcactactgagattttgcccgtttccatgactaagacctccgtggattgcaccatgtacatttgcggtgactccacggaatgctccaacctgcttctgcagtacggttcgttttgcacccagttgaatcgcgcgctgaccggaattgccgtggaacaagacaaaaacactcaggaggttttcgcccaagttaagcagatctacaagacgccccccatcaaagacttcggcggattcaacttctcccagatcttgcccgatccctcgaaaccgtcgaagcgttcgtttatcgaagatctccttttcaacaaagtgacgcttgcggatgctggtttcatcaaacaatacggtgactgcttgggagacattgccgctcgtgacctcatttgcgcgcaaaagtttaatggcctcactgtgctgcccccgctgttgacggatgaaatgattgctcagtacacgtccgcgcttctggcgggaactattacgtcgggctggacgtttggtgctggcgcggcgctccaaatccccttcgcgatgcaaatggcctaccgtttcaacggtatcggagtgactcaaaacgtcttgtacgagaatcagaagctgatcgctaaccagtttaattccgcgattggtaagattcaagattcgctttcctcgacggcttccgctcttggcaagctgcaagacgtcgttaaccaaaacgctcaagctcttaacaccctggttaagcaactgtcgtccaactttggcgccatttcgtcggtgcttaacgacattctctcccgtcttgataaagtcgaggctgaagtgcagatcgatcgtctcatcactggtcgtcttcagtcgcttcagacgtacgtcacccaacagcttatccgtgctgctgaaatccgtgcgtcggctaatcttgcggctacgaagatgtccgagtgcgtcctgggccagtccaagcgtgtcgacttctgtggtaaaggttaccacctcatgtcgtttccccaatcggctccgcacggtgtggtgttcctccacgtgacctacgtgccggcccaagagaaaaactttacgacggcgcccgctatttgccacgacggtaaagcgcacttcccgcgtgagggagttttcgtttccaacggcacccactggttcgtgacccaacgtaatttttacgagccgcaaatcatcactacggacaatactttcgtgtccggaaactgcgacgttgttattggcattgtcaacaataccgtgtacgatccgctgcagccggagcttgactccttcaaggaagaattggacaaatactttaagaatcacacctcgcccgatgtggacttgggcgacatctccggaattaatgcgtccgttgttaacattcagaaggagatcgaccgtctgaacgaagtggcgaagaacctcaacgaatccctgattgacctgcaagagttgggaaaatacgagcaatacattaaatggccgtggtacatctggctgggatttattgctggccttatcgctatcgtcatggtgaccattatgctctgttgcatgacttcgtgttgctcgtgtcttaagggctgctgctcctgtggatcctgctgcaaatttgacgaagacgactcggaaccggtgctgaaaggtgtcaaactgcactacactgaaaacctctacttccagggacaccatcaccatcaccattgaagataagaatatctcattgtgaacatctatgatttaccaattttattctttgtttacagttagacgccagtaattgtgctgtttctctcaagtctgtgtcaatacaaactacgaaacttggcaatttttctcttgaatatgagcacgagattgaaacgcacaaaggaaattagtttccatcctttgacaaagtttgttgctgtttagagaacagatgtcaaaattaacgtgccatggaattgaaccatggtgcgctatccccaaatcacgtcgtttgacctcgtcacatttaaggtatatcaagcatattcacttatatcttgacatcctccctgcttgatattcctttgcccttgagccatcttccccacacgctggagatgacccctgcccctcctttctttatccacccgagtaaggtgtcgacaagtcactttgcccctgagccatcctccccactcaggagatgacccctacccctttcttctttatccaccccatcttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagaggcacttcgagctgtaagtacatcaccgacgagcaaggcaagacgatccgcgcctgttttattgagaacgttgttcgtgttggcctcaatggtagcgatgcgtcattcagcgaagttctggtgctgatgatgtggttcgctttgccactggtcaatgtggtaagcccgtgtaatgtcagtaacctttttactgatctcagcttgagcacggtcgctgatgagcttatccatggccccacggtaacggatatgatcctctagggcgttgacaaattctttgtcggttttcatggggtagacgtcagtgaccagggaacgtctccctacaaaaagttgcgcatactttgctccgttgtcgacggcaggggtgtcggaccagattgtatctgtggcaacggcctcattgcgtcaatggacctttaaagccgggaaacgagacttgaaatgtttacgtagaggtgcattatagacctcacgcgcatattgagtggttgcaaaaatggttttgcgaacggtatcactggagacccatgccaggcaaggacggagggcatcataatcatgttcattgcgttggacagcatgcttgttacacgtaagtatacggtcgagagcatcgtgacgttgagaattacagatggcaacgtgtcggtgcatgtattggccgagattgtcaaatcgtggctcaatatatggcatgccaggtaagtcatggatgtcggtatgccattcagccttcatgtcgatttcgttgtcgagaactgatgggtcccagtcaacgtcagaagtaagaacaacatggggaagggaatgaagttcatgactggtgggtgcgcgcatatccatgtacggaagaccctgtcgtatgtttagtgggatgatgtaatcatccagggtaacaatacgttggttgccgcccacggtacgagaccgatcttgaacacaattgtggcaatgttcaagttgggcgctggaatggatggttttacctttaccaagatgcgcgtattgatgcatgataacaacaatgggccctcgttgcgattcgacaagaccggcagccgtgacaatgtctagattggacaaggtatgttcatttataccggttatgttggctgaacgtccagttttgtgcagtacggtaacatcactccccgcaagtccaccgttggcaccgcgatcgacaagtgcagatgtggtattttgtactgtgtgtcgggagacttggtactgtaacacatttgactgaagtgacttggtggtaccatgaggacggagggaacccgttgatacggtgttgcgtgacgctgcaagtaccttacgtatgtcgcctggatccatacgtccaacccgatcagaaagatgtgctaggagttccgtttctggtgcacaatcatgaaaggtatccacgggagttccattgttgtcgtccggggctgcgtcgcgactgccagttgcatgtacatgcgccatcaagggacgtgccaatccattactaaccgggagaaccttagctggggcagccaatccctgaagtatagccttagcatcatctgagagctggttccacatatctctagggatataaggtcgttcacggttagtgggcgtactatgatgtcggttattagtacaattctccctgcgggcatgcgcattggcttcatagagtacggaaggtgacaagtcaatattgtagttaacatccggatcagtgtcaaagtctgtaggatggtaagaaagatcagtagaatgaatactacgcttttccttgggactacgggaattagagaagttgtttcctttattgtagagtgacgctgaagcaagtagaagactaaggtaactctcgtagctaataggattacctcctttggctaggtcaagagtggctgtgatcttcacttgacaaagttccggtacattgtggacagcattctccaaaagactaagacacagttgctttgggagttgctcagccattggtacagtattgtggtagatacaaaggtggttcttccaatgaaggataaatccttctgctgtgcctgtccatgaggatccatatttcgccgtagttaggtaaccaagcgtggtggctgaactgatcttcgcacttgctgattccgtatagtgtttgacaactttacaaaacacttcttgcgcagttcgctctagcctggacagagaagggacaatcttcgtcgttaagactcgtgcgaatagcaaaagatcacaaaatagcacatcggcaccgaccaacgattatttccaaggaaaaaaagaatgcttcactacaagaaattgtgtcatccctatacagagtcttgttactgtgacagaaaattgatggaagatgtggcggattgcctttacactagccaacttgttcgactaattgcagcttcttctgagaggcttcaccgagtaacgcgaagaacaccggtgtctcgtacatgctcgtcggtgaacgctcgtccaatgacaccccccactttgtatcaatatcccaacttggtagtgaactggaatgatacatgcaatttcgcgccgcatcaacagccacgggccaccatcgacgaatagactcgg

SEQ ID NO: 5 - DNA, 22663 bp, synthetic construct
  source 1..22663: mol_type = other DNA
tcgagctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttaccttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatcctagtacactctatatttttttatgcctcggtaatgattttcattttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagttttacacaggagtctggacttgacgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagcaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgacctcgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgcaaggggttcgcgtcagcgggtgttggcgggtgtcggggctggcttaactatgcggcatcagagcagattgtactgagagtgcaccatatgcggtgtgaaataccacacagatgcgtaaggagaaaataccgcatcaggcgccattcgccattcagctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccggggatcctctagagtcgacctgcaggcatgcaagcttgagtattctatagtctcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgtgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagcccatcgatactagcttgattgggatatctcgctcatgtttgtcgcgtgctatgtctttttaggtactttgaacctacgttcgtacttgtataatatgatcatcgtattatcgtttttcatccgtccagcgcaaaatgcattagcagctagtcctagcgtgcggagctacctggacaggtgcatgacggatgcgtgtccttcagtgactttctaattaacagtaacttctttacttatgtttcagtttgtaagaagcgggattcgctcgtcgcttgacatctgattggactgcgtcggcacgtgaaaactacattgtgaaatctgctaaaactccgggtatctctgacacaaaacgattcggcttcgcaatttcaacattacggtcaaggctaacgtatctttctcggtcaacttcagattatgccgattaaattgtcgtagctttcaaggcgttttgagtactgcggcagttgttgaacctgcaaggagaagatctcgacaacagaataaagcgaaaaatgggtctcatgcactaacactcaggcctccctcataatctctgtttgagtttaccaacaacacatatatacatttcgacaaaatgaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctgaccgacgccgaccaacaccgccggtccgacgcggcccgacgggtccgaggcctcggagatctgggcccatgcggccgcaacaactacctcgactttggctgggacactttcagtgaggacaagaagcttcagaagcgtgctatcgaactcaaccagggacgtgcggcacaaatgggcatccttgctctcatggtgcacgaacagttgggagtctctatccttccttaaaaatttaattttcattagttgcagtcactccgctttggtttctcgcgttttacgcgccgtcagtttcttggcccacgaactggattcccacttgttcatggtgtcggcttccgtccaggccttgagcagactcttttgacgagcgtttcgctgaatatcgaccttcaggtcggtcagcgcgaggcgtttgtaggaaataacttgtcgggtgactcccgtgatagattctgggccgtcaatcagacacttgttctggtcgacaatgtcaataatggtggcgagcttgccagcctccggtccgtagttgatcaaaaccacgcgacccaattcaacgtatcggtgaaacggcatggttgttaaagagattgaaaatgagagagaagtggtatgtgtatgtaaatgtgtgggtgatcctacgcaaaaacgagtttcgaagtagcagcgacacacgagatggagtgtagtttgttgttggtcaaaggacacggcgaggggcccaccacgagttgtccgtagtccgtttttgtcgggtaatttcgcatgctagagacggagcacggggttccgcgagcggcggatacccgcgtgcggtgatgcgggccgagaattttagtaaaccggtaatggagggttgaatagggtagggcgggaaaagaccaccaacctaccccggaaacgataggacatgtcgtagcggacatttaaaagaagagaatttcgttggatatacctacttcttggatttacatcacacaaccagtagaagataccgggtagagtaggtactcttcttcgacaccacacaaaacaccaaccaatacactctggaaaagacacttctaacgtgaaaggagatcatcgtccccacaatttgccgagtgatattgttcgcttcgcaacaacctagcagactctgctggtgtcacattttcggacacattttcgtctgccaaggccgcgtacacacacgcacatattcataaaactcgttcggaaatcgtttcatgtcgagttttccgtatcccgcctttctccaccaccgtccggtggacgccgcggcgcagcgtgccgcactcgcggccgttggtcgttgccgcatcgtccctacatcgtcctttcctggggccgctgactttcgcgagattgtccaaatgaacgtggcacaagcacacgtcggactcacggacgagaccgacggtgcggatcaggaaacgctccagaccacacgcaaggcccggatgaaacagggcatcgccatggccaagacgtatctcgtcgaaaccttacagttggatgtcacggtcgtggatgtggaagtcgttttggatgctcgggatgtgattcaatcggacggctgtctcgcgagtggactcctcgatgcgggttgtgacaaaattgtcgtggacggcaccaatcgggacgcttttgagactgcacaaattccgaaagcgagactgatgggccactttgtgtatccccataccgattcacgaacccgaaacgggagtctcgccgtggcctttgccgaagtccaacccttttgcgattccgtcagtgtcgagctatcgaatcctgaagccgtcacggaaagtgtcatccaggaaattgtgcaagcggcaaaagcccagcaattgcaaatttcaatcgtctacaatcccaacgactacgagaatcctggtgatcacgccgccatggttggtcgcgtatgccagagcttcccagaagggcaagtccggttggccttgaccgatccgaccgcctcgcaactcggacacgcctacgcggcgtgtctcaaaaccgaccgggacgacggtctctttaccaccgtcgtctgtactcgcaacaatgaagcactggggcttgtctactcctccacgtcgtctattgtcgccgcactggaatgtggtcggggtgtctactacagccgttcccggaacggattgtggcgcaagggcgacacgtccggacactttcaggtacttcaccggctcgacaaagactgtgacggggatgctttgcgggtgacggtcactcaaaaaggcacccctgccgcattttgccacttgaatactctaacgtgctggggtacgtcgagaggcttgcgacatttggaagaaacgctgcaagaccgactcgtgtcggctccggaaggatcctacacgaaacgtctgtttcaagacgagacactgttgcgggacaagctggtcgaagaagcgcaggaattgtcggaagccgatacgaaggcacacgtggcggaagaactggcggatgtcctctactttgccatggtgagggcggtcaaggctggagtcagtttggatgatgccgtcgcagagctagattctagggcgcgaaaagtgacgcgacgaaaaggcgatagtaaagcctttcgaattgcagcgggtgaagctattttgaaaaaagcgcagtcgtagcaacgcaagaaatagattcgtatacggaatctaggatacctccttggatattaatagcacatttctgtccttggtagaatcttgagtgttgcagcggaacgataaagggtcgaatggtcaggctgagctgaatagtccccactgagttctatccggaacggacttgcgacgaggagctgccaaagtgcaggcgtgcttccattgagtcccaacctatgctgctagcggtgtgttcgcttttagaccttgaccggggtccagacttgtctaccgaagcagggtactccgactcatccactagatgaagcggcgcatctagaaatgctccattgggaaacaaatcgtccaagctatcaaaactggatgagacactggccaacgaagtcatctcggaaatcccatcattgtattcgactgaccctccgactagccagttgcgtttgggaatttccatgtgatttggaaggcgaatcaagcgtgggaccaagaatcgatgaggagaaagcggagggagacgcctgtcatctttatgtaatcgcgggggattggtgactttgtcagcgttgatgttaccgtctgcggcgatcaaaagtgtcgcttggtcaatgtcgcttgtcaaggtcgaggtgtacgaaagttctggactatcagcaccactctgttgggcgcctcgagactggacaaagtcctctgaatttttcaaaggtaaaccaagctgtaataagttgttgcgaacggcggcctgcaagcggccgtacatagcggtcccagggagagggcttggtcgagtgggaacggcatctcgctgctgctctgtcttgttcattgtcccagatgttgccgtttgagtaaagggatgcttaattttaccacatgctgtgtctttgagctgaacaggggtgtgtttgaatgattccatgtcgctgccaccatgacatgagcaactctcgcggggctttgtagtgctccctttcccagctactcgacgcatcaggcgggcggattgatagtgaaaaccttattcattgtcagagcttaagccggtctatctttccactgtcaaacagctcttgattgtcgcccgcgcgaaaatagtagcactaactgtaacttcaaaatacaaaatgttctctgttaccatacagtgaatgtaactttcgaattgacagtattagtagtcgtattgacagtgaggcacgcccctcaatgtgcgaggtggaaaatataccagcatgacaatgaatcttggagattcttttgctgtcatcaagattcaccgccaaatcttcaggaacctatcacgtccacaggcgatgttaattcttgagtcgtcaaaacaaagtcctgtcctacctgtagaagttgacagcgagcaattgtatgcaaacttctgacttatgttataataacattaaaggtaattaagtatcttcaatttggcattttgtcactgtcagtccgttccgacaatacaggtagatttggaatgaatcttttctatgctgctgcgaatcttgtacaccttcgaggccgtagattctgtccgacgaagcgataattattgcaaaatacatggactcattattttgattcgatttctttttggtatccgactcgaaaagatccatcacggcgagcatgaatcttcgttgtatccttccgtttctcctcgcaagcttctcggctggggcttgcgtcaatctgactactcgtacccagcttccgcccgcttacaccaactctttcacccgaggcgtttactacccggacaaagttttccgttcctcggtccttcactcgacgcaggacctttttttgccgttcttttcgaacgtcacttggttccacgcgattcacgtttccggcacgaatggtacgaaacgttttgataatcccgttctgccctttaatgacggtgtctacttcgcgtccactgaaaaatcgaacatcatccgtggctggatctttggcactacgctcgattccaaaacccaatcgctgctcatcgttaataatgccaccaatgtcgttattaaagtctgcgagttccaattttgtaatgacccgtttcttggtgtgtactaccacaagaacaacaagtcgtggatggagtccgagttccgtgtgtactcctccgccaacaactgtacttttgaatacgtttcgcaaccctttcttatggatttggaaggcaaacagggcaactttaaaaatctgcgtgaatttgtcttcaagaatattgacggttacttcaaaatttactccaaacacactccgattaacttggtgcgcgatcttccccaaggtttctccgcgctcgaaccgctggtcgaccttccgatcggtatcaacattacccgcttccagactctcctggcgcttcaccgttcgtacctgactcccggtgattcgtcctcgggttggacggcgggtgcggcggcttactacgttggctaccttcaaccgcgcacgtttttgttgaagtacaacgagaacggtaccatcactgatgcggtggactgcgctttggacccgctttccgagaccaaatgtactcttaagtcctttacggtcgagaaaggtatctaccaaacttctaatttccgtgtgcagcccactgagtccatcgtgcgcttcccgaatattacgaatctttgcccctttggagaagtcttcaatgccactcgtttcgcctccgtttacgcctggaaccgtaagcgcatctccaattgcgtcgccgattactccgttctttacaactcggcttcgttttcgactttcaaatgctacggtgtgtccccgactaagctcaacgacctctgttttacgaatgtgtacgctgattcctttgtcattcgtggtgacgaagtccgccagatcgctccgggtcagaccggaaagattgcggactacaactacaaactgccggacgacttcactggatgcgtgattgcctggaattccaataacttggattcgaaagttggaggtaattacaattacttgtaccgtcttttccgcaagtcgaaccttaaaccctttgaacgtgacatttccaccgaaatttaccaagccggctcgaccccctgtaacggagtggaaggctttaattgctacttcccccttcaatcgtacggattccagccgactaacggtgttggataccaaccctaccgtgtggtggtcctgtccttcgaactgctccacgctccggcgactgtctgtggacccaagaaatcgaccaatctggtcaagaataaatgtgtcaattttaattttaacggtttgacgggaacgggtgtgctcaccgagtccaacaaaaagttccttcccttccagcagtttggtcgcgatattgcggataccacggacgcggtgcgtgatccgcaaaccctggaaattttggatattactccctgctcctttggtggtgtttccgtcattaccccgggcaccaatacttcgaatcaggttgcggtcctctaccaggacgtgaattgcactgaagtccccgtcgccattcacgccgaccagcttactccgacgtggcgcgtgtactccaccggctccaatgtttttcaaacccgtgcgggctgcctgatcggcgccgagcacgttaacaattcctacgagtgcgacatccccatcggagccggcatctgcgcttcgtaccagactcaaacgaattcgccgcgtcgcgcccgttccgtggcgtcccagtcgattattgcgtacaccatgtccctgggagcggaaaactccgttgcctactcgaataattcgattgcgatccccaccaacttcactatctcggtcactactgagattttgcccgtttccatgactaagacctccgtggattgcaccatgtacatttgcggtgactccacggaatgctccaacctgcttctgcagtacggttcgttttgcacccagttgaatcgcgcgctgaccggaattgccgtggaacaagacaaaaacactcaggaggttttcgcccaagttaagcagatctacaagacgccccccatcaaagacttcggcggattcaacttctcccagatcttgcccgatccctcgaaaccgtcgaagcgttcgtttatcgaagatctccttttcaacaaagtgacgcttgcggatgctggtttcatcaaacaatacggtgactgcttgggagacattgccgctcgtgacctcatttgcgcgcaaaagtttaatggcctcactgtgctgcccccgctgttgacggatgaaatgattgctcagtacacgtccgcgcttctggcgggaactattacgtcgggctggacgtttggtgctggcgcggcgctccaaatccccttcgcgatgcaaatggcctaccgtttcaacggtatcggagtgactcaaaacgtcttgtacgagaatcagaagctgatcgctaaccagtttaattccgcgattggtaagattcaagattcgctttcctcgacggcttccgctcttggcaagctgcaagacgtcgttaaccaaaacgctcaagctcttaacaccctggttaagcaactgtcgtccaactttggcgccatttcgtcggtgcttaacgacattctctcccgtcttgataaagtcgaggctgaagtgcagatcgatcgtctcatcactggtcgtcttcagtcgcttcagacgtacgtcacccaacagcttatccgtgctgctgaaatccgtgcgtcggctaatcttgcggctacgaagatgtccgagtgcgtcctgggccagtccaagcgtgtcgacttctgtggtaaaggttaccacctcatgtcgtttccccaatcggctccgcacggtgtggtgttcctccacgtgacctacgtgccggcccaagagaaaaactttacgacggcgcccgctatttgccacgacggtaaagcgcacttcccgcgtgagggagttttcgtttccaacggcacccactggttcgtgacccaacgtaatttttacgagccgcaaatcatcactacggacaatactttcgtgtccggaaactgcgacgttgttattggcattgtcaacaataccgtgtacgatccgctgcagccggagcttgactccttcaaggaagaattggacaaatactttaagaatcacacctcgcccgatgtggacttgggcgacatctccggaattaatgcgtccgttgttaacattcagaaggagatcgaccgtctgaacgaagtggcgaagaacctcaacgaatccctgattgacctgcaagagttgggaaaatacgagcaatacattaaatggccgtggtacatctggctgggatttattgctggccttatcgctatcgtcatggtgaccattatgctctgttgcatgacttcgtgttgctcgtgtcttaagggctgctgctcctgtggatcctgctgcaaatttgacgaagacgactcggaaccggtgctgaaaggtgtcaaactgcactacactgaaaacctctacttccagggacaccatcaccatcaccattgaagataagaatatctcattgtgaacatctatgatttaccaattttattctttgtttacagttagacgccagtaattgtgctgtttctctcaagtctgtgtcaatacaaactacgaaacttggcaatttttctcttgaatatgagcacgagattgaaacgcacaaaggaaattagtttccatcctttgacaaagtttgttgctgtttagagaacagatgtcaaaattaacgtgccatggaattgaaccatggtgcgctatccccaaatcacgtcgtttgacctcgtcacatttaaggtatatcaagcatattcacttatatcttgacatcctccctgcttgatattcctttgcccttgagccatcttccccacacgctggagatgacccctgcccctcctttctttatccacccgagtaaggtgtcgacaagtcactttgcccctgagccatcctccccactcaggagatgacccctacccctttcttctttatccaccccatcttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagaggcacttcgagctgtaagtacatcaccgacgagcaaggcaagacgatccgcgcctgttttattgagaacgttgttcgtgttggcctcaatggtagcgatgcgtcattcagcgaagttctggtgctgatgatgtggttcgctttgccactggtcaatgtggtaagcccgtgtaatgtcagtaacctttttactgatctcagcttgagcacggtcgctgatgagcttatccatggccccacggtaacggatatgatcctctagggcgttgacaaattctttgtcggttttcatggggtagacgtcagtgaccagggaacgtctccctacaaaaagttgcgcatactttgctccgttgtcgacggcaggggtgtcggaccagattgtatctgtggcaacggcctcattgcgtcaatggacctttaaagccgggaaacgagacttgaaatgtttacgtagaggtgcattatagacctcacgcgcatattgagtggttgcaaaaatggttttgcgaacggtatcactggagacccatgccaggcaaggacggagggcatcataatcatgttcattgcgttggacagcatgcttgttacacgtaagtatacggtcgagagcatcgtgacgttgagaattacagatggcaacgtgtcggtgcatgtattggccgagattgtcaaatcgtggctcaatatatggcatgccaggtaagtcatggatgtcggtatgccattcagccttcatgtcgatttcgttgtcgagaactgatgggtcccagtcaacgtcagaagtaagaacaacatggggaagggaatgaagttcatgactggtgggtgcgcgcatatccatgtacggaagaccctgtcgtatgtttagtgggatgatgtaatcatccagggtaacaatacgttggttgccgcccacggtacgagaccgatcttgaacacaattgtggcaatgttcaagttgggcgctggaatggatggttttacctttaccaagatgcgcgtattgatgcatgataacaacaatgggccctcgttgcgattcgacaagaccggcagccgtgacaatgtctagattggacaaggtatgttcatttataccggttatgttggctgaacgtccagttttgtgcagtacggtaacatcactccccgcaagtccaccgttggcaccgcgatcgacaagtgcagatgtggtattttgtactgtgtgtcgggagacttggtactgtaacacatttgactgaagtgacttggtggtaccatgaggacggagggaacccgttgatacggtgttgcgtgacgctgcaagtaccttacgtatgtcgcctggatccatacgtccaacccgatcagaaagatgtgctaggagttccgtttctggtgcacaatcatgaaaggtatccacgggagttccattgttgtcgtccggggctgcgtcgcgactgccagttgcatgtacatgcgccatcaagggacgtgccaatccattactaaccgggagaaccttagctggggcagccaatccctgaagtatagccttagcatcatctgagagctggttccacatatctctagggatataaggtcgttcacggttagtgggcgtactatgatgtcggttattagtacaattctccctgcgggcatgcgcattggcttcatagagtacggaaggtgacaagtcaatattgtagttaacatccggatcagtgtcaaagtctgtaggatggtaagaaagatcagtagaatgaatactacgcttttccttgggactacgggaattagagaagttgtttcctttattgtagagtgacgctgaagcaagtagaagactaaggtaactctcgtagctaataggattacctcctttggctaggtcaagagtggctgtgatcttcacttgacaaagttccggtacattgtggacagcattctccaaaagactaagacacagttgctttgggagttgctcagccattggtacagtattgtggtagatacaaaggtggttcttccaatgaaggataaatccttctgctgtgcctgtccatgaggatccatatttcgccgtagttaggtaaccaagcgtggtggctgaactgatcttcgcacttgctgattccgtatagtgtttgacaactttacaaaacacttcttgcgcagttcgctctagcctggacagagaagggacaatcttcgtcgttaagactcgtgcgaatagcaaaagatcacaaaatagcacatcggcaccgaccaacgattatttccaaggaaaaaaagaatgcttcactacaagaaattgtgtcatccctatacagagtcttgttactgtgacagaaaattgatggaagatgtggcggattgcctttacactagccaacttgttcgactaattgcagcttcttctgagaggcttcaccgagtaacgcgaagaacaccggtgtctcgtacatgctcgtcggtgaacgctcgtccaatgacaccccccactttgtatcaatatcccaacttggtagtgaactggaatgatacatgcaatttcgcgccgcatcaacagccacgggccaccatcgacgaatagactcgg

SEQ ID NO: 6 - DNA, 22748 bp, synthetic construct
  source 1..22748: mol_type = other DNA
tcgagctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttaccttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatcctagtacactctatatttttttatgcctcggtaatgattttcattttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagttttacacaggagtctggacttgacgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagcaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgacctcgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgcaaggggttcgcgtcagcgggtgttggcgggtgtcggggctggcttaactatgcggcatcagagcagattgtactgagagtgcaccatatgcggtgtgaaataccacacagatgcgtaaggagaaaataccgcatcaggcgccattcgccattcagctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccggggatcctctagagtcgacctgcaggcatgcaagcttgagtattctatagtctcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgtgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagcccatcgatactagcttgattgggatatctcgctcatgtttgtcgcgtgctatgtctttttaggtactttgaacctacgttcgtacttgtataatatgatcatcgtattatcgtttttcatccgtccagcgcaaaatgcattagcagctagtcctagcgtgcggagctacctggacaggtgcatgacggatgcgtgtccttcagtgactttctaattaacagtaacttctttacttatgtttcagtttgtaagaagcgggattcgctcgtcgcttgacatctgattggactgcgtcggcacgtgaaaactacattgtgaaatctgctaaaactccgggtatctctgacacaaaacgattcggcttcgcaatttcaacattacggtcaaggctaacgtatctttctcggtcaacttcagattatgccgattaaattgtcgtagctttcaaggcgttttgagtactgcggcagttgttgaacctgcaaggagaagatctcgacaacagaataaagcgaaaaatgggtctcatgcactaacactcaggcctccctcataatctctgtttgagtttaccaacaacacatatatacatttcgacaaaatgaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctgaccgacgccgaccaacaccgccggtccgacgcggcccgacgggtccgaggcctcggagatctgggcccatgcggccgcaacaactacctcgactttggctgggacactttcagtgaggacaagaagcttcagaagcgtgctatcgaactcaaccagggacgtgcggcacaaatgggcatccttgctctcatggtgcacgaacagttgggagtctctatccttccttaaaaatttaattttcattagttgcagtcactccgctttggtttctcgcgttttacgcgccgtcagtttcttggcccacgaactggattcccacttgttcatggtgtcggcttccgtccaggccttgagcagactcttttgacgagcgtttcgctgaatatcgaccttcaggtcggtcagcgcgaggcgtttgtaggaaataacttgtcgggtgactcccgtgatagattctgggccgtcaatcagacacttgttctggtcgacaatgtcaataatggtggcgagcttgccagcctccggtccgtagttgatcaaaaccacgcgacccaattcaacgtatcggtgaaacggcatggttgttaaagagattgaaaatgagagagaagtggtatgtgtatgtaaatgtgtgggtgatcctacgcaaaaacgagtttcgaagtagcagcgacacacgagatggagtgtagtttgttgttggtcaaaggacacggcgaggggcccaccacgagttgtccgtagtccgtttttgtcgggtaatttcgcatgctagagacggagcacggggttccgcgagcggcggatacccgcgtgcggtgatgcgggccgagaattttagtaaaccggtaatggagggttgaatagggtagggcgggaaaagaccaccaacctaccccggaaacgataggacatgtcgtagcggacatttaaaagaagagaatttcgttggatatacctacttcttggatttacatcacacaaccagtagaagataccgggtagagtaggtactcttcttcgacaccacacaaaacaccaaccaatacactctggaaaagacacttctaacgtgaaaggagatcatcgtccccacaatttgccgagtgatattgttcgcttcgcaacaacctagcagactctgctggtgtcacattttcggacacattttcgtctgccaaggccgcgtacacacacgcacatattcataaaactcgttcggaaatcgtttcatgtcgagttttccgtatcccgcctttctccaccaccgtccggtggacgccgcggcgcagcgtgccgcactcgcggccgttggtcgttgccgcatcgtccctacatcgtcctttcctggggccgctgactttcgcgagattgtccaaatgaacgtggcacaagcacacgtcggactcacggacgagaccgacggtgcggatcaggaaacgctccagaccacacgcaaggcccggatgaaacagggcatcgccatggccaagacgtatctcgtcgaaaccttacagttggatgtcacggtcgtggatgtggaagtcgttttggatgctcgggatgtgattcaatcggacggctgtctcgcgagtggactcctcgatgcgggttgtgacaaaattgtcgtggacggcaccaatcgggacgcttttgagactgcacaaattccgaaagcgagactgatgggccactttgtgtatccccataccgattcacgaacccgaaacgggagtctcgccgtggcctttgccgaagtccaacccttttgcgattccgtcagtgtcgagctatcgaatcctgaagccgtcacggaaagtgtcatccaggaaattgtgcaagcggcaaaagcccagcaattgcaaatttcaatcgtctacaatcccaacgactacgagaatcctggtgatcacgccgccatggttggtcgcgtatgccagagcttcccagaagggcaagtccggttggccttgaccgatccgaccgcctcgcaactcggacacgcctacgcggcgtgtctcaaaaccgaccgggacgacggtctctttaccaccgtcgtctgtactcgcaacaatgaagcactggggcttgtctactcctccacgtcgtctattgtcgccgcactggaatgtggtcggggtgtctactacagccgttcccggaacggattgtggcgcaagggcgacacgtccggacactttcaggtacttcaccggctcgacaaagactgtgacggggatgctttgcgggtgacggtcactcaaaaaggcacccctgccgcattttgccacttgaatactctaacgtgctggggtacgtcgagaggcttgcgacatttggaagaaacgctgcaagaccgactcgtgtcggctccggaaggatcctacacgaaacgtctgtttcaagacgagacactgttgcgggacaagctggtcgaagaagcgcaggaattgtcggaagccgatacgaaggcacacgtggcggaagaactggcggatgtcctctactttgccatggtgagggcggtcaaggctggagtcagtttggatgatgccgtcgcagagctagattctagggcgcgaaaagtgacgcgacgaaaaggcgatagtaaagcctttcgaattgcagcgggtgaagctattttgaaaaaagcgcagtcgtagcaacgcaagaaatagattcgtatacggaatctaggatacctccttggatattaatagcacatttctgtccttggtagaatcttgagtgttgcagcggaacgataaagggtcgaatggtcaggctgagctgaatagtccccactgagttctatccggaacggacttgcgacgaggagctgccaaagtgcaggcgtgcttccattgagtcccaacctatgctgctagcggtgtgttcgcttttagaccttgaccggggtccagacttgtctaccgaagcagggtactccgactcatccactagatgaagcggcgcatctagaaatgctccattgggaaacaaatcgtccaagctatcaaaactggatgagacactggccaacgaagtcatctcggaaatcccatcattgtattcgactgaccctccgactagccagttgcgtttgggaatttccatgtgatttggaaggcgaatcaagcgtgggaccaagaatcgatgaggagaaagcggagggagacgcctgtcatctttatgtaatcgcgggggattggtgactttgtcagcgttgatgttaccgtctgcggcgatcaaaagtgtcgcttggtcaatgtcgcttgtcaaggtcgaggtgtacgaaagttctggactatcagcaccactctgttgggcgcctcgagactggacaaagtcctctgaatttttcaaaggtaaaccaagctgtaataagttgttgcgaacggcggcctgcaagcggccgtacatagcggtcccagggagagggcttggtcgagtgggaacggcatctcgctgctgctctgtcttgttcattgtcccagatgttgccgtttgagtaaagggatgcttaattttaccacatgctgtgtctttgagctgaacaggggtgtgtttgaatgattccatgtcgctgccaccatgacatgagcaactctcgcggggctttgtagtgctccctttcccagctactcgacgcatcaggcgggcggattgatagtgaaaaccttattcattgtcagagcttaagccggtctggtctatctttccactgtcaaacagctcttgattgtcgcccgcgcgaaaatagtagcactaactgtaacttcaaaatacaaaatgttctctgttaccatacagtgaatgtaactttcgaattgacagtattagtagtcgtattgacagtgaggcacgcccctcaatgtgcgaggtggaaaatataccagcatgacaatgaatcttggagattcttttgctgtcatcaagattcaccgccaaatcttcaggaacctatcacgtccacaggcgatgttaattcttgagtcgtcaaaacaaagtcctgtcctacctgtagaagttgacagcgagcaattgtatgcaaacttctgactttgttataataacattaaaggtaattaagtatcttcaattaggcattttgtcactgtcagtccgttccgacaatataggtagatttggaatgaatcttttctatgctgctgcgaatcttgtacacctttgaggccgtagattctgtccgacgaagcgataattattgcaaaatacatggactcattattttgattcgatttctttttggtatccgactcgaaaagatccatcacggcgagcatgaatcttcgttgtatccttccgtttctcctcgcaagcttctcggctggggcttgcgtcaatctgactactcgtacccagcttccgcccgcttacaccaactctttcacccgaggcgtttactacccggacaaagttttccgttcctcggtccttcactcgacgcaggacctttttttgccgttcttttcgaacgtcacttggttccacgcgattcacgtttccggcacgaatggtacgaaacgttttgataatcccgttctgccctttaatgacggtgtctacttcgcgtccactgaaaaatcgaacatcatccgtggctggatctttggcactacgctcgattccaaaacccaatcgctgctcatcgttaataatgccaccaatgtcgttattaaagtctgcgagttccaattttgtaatgacccgtttcttggtgtgtactaccacaagaacaacaagtcgtggatggagtccgagttccgtgtgtactcctccgccaacaactgtacttttgaatacgtttcgcaaccctttcttatggatttggaaggcaaacagggcaactttaaaaatctgcgtgaatttgtcttcaagaatattgacggttacttcaaaatttactccaaacacactccgattaacttggtgcgcgatcttccccaaggtttctccgcgctcgaaccgctggtcgaccttccgatcggtatcaacattacccgcttccagactctcctggcgcttcaccgttcgtacctgactcccggtgattcgtcctcgggttggacggcgggtgcggcggcttactacgttggctaccttcaaccgcgcacgtttttgttgaagtacaacgagaacggtaccatcactgatgcggtggactgcgctttggacccgctttccgagaccaaatgtactcttaagtcctttacggtcgagaaaggtatctaccaaacttctaatttccgtgtgcagcccactgagtccatcgtgcgcttcccgaatattacgaatctttgcccctttggagaagtcttcaatgccactcgtttcgcctccgtttacgcctggaaccgtaagcgcatctccaattgcgtcgccgattactccgttctttacaactcggcttcgttttcgactttcaaatgctacggtgtgtccccgactaagctcaacgacctctgttttacgaatgtgtacgctgattcctttgtcattcgtggtgacgaagtccgccagatcgctccgggtcagaccggaaagattgcggactacaactacaaactgccggacgacttcactggatgcgtgattgcctggaattccaataacttggattcgaaagttggaggtaattacaattacttgtaccgtcttttccgcaagtcgaaccttaaaccctttgaacgtgacatttccaccgaaatttaccaagccggctcgaccccctgtaacggagtggaaggctttaattgctacttcccccttcaatcgtacggattccagccgactaacggtgttggataccaaccctaccgtgtggtggtcctgtccttcgaactgctccacgctccggcgactgtctgtggacccaagaaatcgaccaatctggtcaagaataaatgtgtcaattttaattttaacggtttgacgggaacgggtgtgctcaccgagtccaacaaaaagttccttcccttccagcagtttggtcgcgatattgcggataccacggacgcggtgcgtgatccgcaaaccctggaaattttggatattactccctgctcctttggtggtgtttccgtcattaccccgggcaccaatacttcgaatcaggttgcggtcctctaccaggacgtgaattgcactgaagtccccgtcgccattcacgccgaccagcttactccgacgtggcgcgtgtactccaccggctccaatgtttttcaaacccgtgcgggctgcctgatcggcgccgagcacgttaacaattcctacgagtgcgacatccccatcggagccggcatctgcgcttcgtaccagactcaaacgaattcgccgcgtcgcgcccgttccgtggcgtcccagtcgattattgcgtacaccatgtccctgggagcggaaaactccgttgcctactcgaataattcgattgcgatccccaccaacttcactatctcggtcactactgagattttgcccgtttccatgactaagacctccgtggattgcaccatgtacatttgcggtgactccacggaatgctccaacctgcttctgcagtacggttcgttttgcacccagttgaatcgcgcgctgaccggaattgccgtggaacaagacaaaaacactcaggaggttttcgcccaagttaagcagatctacaagacgccccccatcaaagacttcggcggattcaacttctcccagatcttgcccgatccctcgaaaccgtcgaagcgttcgtttatcgaagatctccttttcaacaaagtgacgcttgcggatgctggtttcatcaaacaatacggtgactgcttgggagacattgccgctcgtgacctcatttgcgcgcaaaagtttaatggcctcactgtgctgcccccgctgttgacggatgaaatgattgctcagtacacgtccgcgcttctggcgggaactattacgtcgggctggacgtttggtgctggcgcggcgctccaaatccccttcgcgatgcaaatggcctaccgtttcaacggtatcggagtgactcaaaacgtcttgtacgagaatcagaagctgatcgctaaccagtttaattccgcgattggtaagattcaagattcgctttcctcgacggcttccgctcttggcaagctgcaagacgtcgttaaccaaaacgctcaagctcttaacaccctggttaagcaactgtcgtccaactttggcgccatttcgtcggtgcttaacgacattctctcccgtcttgatccgccggaggctgaagtgcagatcgatcgtctcatcactggtcgtcttcagtcgcttcagacgtacgtcacccaacagcttatccgtgctgctgaaatccgtgcgtcggctaatcttgcggctacgaagatgtccgagtgcgtcctgggccagtccaagcgtgtcgacttctgtggtaaaggttaccacctcatgtcgtttccccaatcggctccgcacggtgtggtgttcctccacgtgacctacgtgccggcccaagagaaaaactttacgacggcgcccgctatttgccacgacggtaaagcgcacttcccgcgtgagggagttttcgtttccaacggcacccactggttcgtgacccaacgtaatttttacgagccgcaaatcatcactacggacaatactttcgtgtccggaaactgcgacgttgttattggcattgtcaacaataccgtgtacgatccgctgcagccggagcttgactccttcaaggaagaattggacaaatactttaagaatcacacctcgcccgatgtggacttgggcgacatctccggaattaatgcgtccgttgttaacattcagaaggagatcgaccgtctgaacgaagtggcgaagaacctcaacgaatccctgattgacctgcaagagttgggaaaatacgagcaatacattaaatggccgtggtacatctggctgggatttattgctggccttatcgctatcgtcatggtgaccattgtgctctgttgcatgacttcgtgttgctcgtgtcttaagggctgctgctcctgtggatcctgctgcaaatttgacgaagacgactcggaaccggtgctgaaaggtgtcaaactgcactacactggatacattcccgaagccccccgtgacggacaggcctacgtccgtaaggacggagaatgggtcctcctctccaccttcctcgaaaacctctacttccagggacaccatcaccatcaccattgaagataagaatatctcattgtgaacatctatgatttaccaattttattctttgtttacagttagacgccagtaattgtgctgtttctctcaagtctgtgtcaatacaaactacgaaacttggcaatttttctcttgaatatgagcacgagattgaaacgcacaaaggaaattagtttccatcctttgacaaagtttgttgctgtttagagaacagatgtcaaaattaacgtgccatggaattgaaccatggtgcgctatccccaaatcacgtcgtttgacctcgtcacatttaaggtatatcaagcatattcacttatatcttgacatcctccctgcttgatattcctttgcccttgagccatcttccccacacgctggagatgacccctgcccctcctttctttatccacccgagtaaggtgtcgacaagtcactttgcccctgagccatcctccccactcaggagatgacccctacccctttcttctttatccaccccatcttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagaggcacttcgagctgtaagtacatcaccgacgagcaaggcaagacgatccgcgcctgttttattgagaacgttgttcgtgttggcctcaatggtagcgatgcgtcattcagcgaagttctggtgctgatgatgtggttcgctttgccactggtcaatgtggtaagcccgtgtaatgtcagtaacctttttactgatctcagcttgagcacggtcgctgatgagcttatccatggccccacggtaacggatatgatcctctagggcgttgacaaattctttgtcggttttcatggggtagacgtcagtgaccagggaacgtctccctacaaaaagttgcgcatactttgctccgttgtcgacggcaggggtgtcggaccagattgtatctgtggcaacggcctcattgcgtcaatggacctttaaagccgggaaacgagacttgaaatgtttacgtagaggtgcattatagacctcacgcgcatattgagtggttgcaaaaatggttttgcgaacggtatcactggagacccatgccaggcaaggacggagggcatcataatcatgttcattgcgttggacagcatgcttgttacacgtaagtatacggtcgagagcatcgtgacgttgagaattacagatggcaacgtgtcggtgcatgtattggccgagattgtcaaatcgtggctcaatatatggcatgccaggtaagtcatggatgtcggtatgccattcagccttcatgtcgatttcgttgtcgagaactgatgggtcccagtcaacgtcagaagtaagaacaacatggggaagggaatgaagttcatgactggtgggtgcgcgcatatccatgtacggaagaccctgtcgtatgtttagtgggatgatgtaatcatccagggtaacaatacgttggttgccgcccacggtacgagaccgatcttgaacacaattgtggcaatgttcaagttgggcgctggaatggatggttttacctttaccaagatgcgcgtattgatgcatgataacaacaatgggccctcgttgcgattcgacaagaccggcagccgtgacaatgtctagattggacaaggtatgttcatttataccggttatgttggctgaacgtccagttttgtgcagtacggtaacatcactccccgcaagtccaccgttggcaccgcgatcgacaagtgcagatgtggtattttgtactgtgtgtcgggagacttggtactgtaacacatttgactgaagtgacttggtggtaccatgaggacggagggaacccgttgatacggtgttgcgtgacgctgcaagtaccttacgtatgtcgcctggatccatacgtccaacccgatcagaaagatgtgctaggagttccgtttctggtgcacaatcatgaaaggtatccacgggagttccattgttgtcgtccggggctgcgtcgcgactgccagttgcatgtacatgcgccatcaagggacgtgccaatccattactaaccgggagaaccttagctggggcagccaatccctgaagtatagccttagcatcatctgagagctggttccacatatctctagggatataaggtcgttcacggttagtgggcgtactatgatgtcggttattagtacaattctccctgcgggcatgcgcattggcttcatagagtacggaaggtgacaagtcaatattgtagttaacatccggatcagtgtcaaagtctgtaggatggtaagaaagatcagtagaatgaatactacgcttttccttgggactacgggaattagagaagttgtttcctttattgtagagtgacgctgaagcaagtagaagactaaggtaactctcgtagctaataggattacctcctttggctaggtcaagagtggctgtgatcttcacttgacaaagttccggtacattgtggacagcattctccaaaagactaagacacagttgctttgggagttgctcagccattggtacagtattgtggtagatacaaaggtggttcttccaatgaaggataaatccttctgctgtgcctgtccatgaggatccatatttcgccgtagttaggtaaccaagcgtggtggctgaactgatcttcgcacttgctgattccgtatagtgtttgacaactttacaaaacacttcttgcgcagttcgctctagcctggacagagaagggacaatcttcgtcgttaagactcgtgcgaatagcaaaagatcacaaaatagcacatcggcaccgaccaacgattatttccaaggaaaaaaagaatgcttcactacaagaaattgtgtcatccctatacagagtcttgttactgtgacagaaaattgatggaagatgtggcggattgcctttacactagccaacttgttcgactaattgcagcttcttctgagaggcttcaccgagtaacgcgaagaacaccggtgtctcgtacatgctcgtcggtgaacgctcgtccaatgacaccccccactttgtatcaatatcccaacttggtagtgaactggaatgatacatgcaatttcgcgccgcatcaacagccacgggccaccatcgacgaatagactcgg

SEQ ID NO: 7 - DNA, 22744 bp, synthetic construct
  source 1..22744: mol_type = other DNA
tcgagctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttaccttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatcctagtacactctatatttttttatgcctcggtaatgattttcattttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagttttacacaggagtctggacttgacgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagcaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgacctcgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgcaaggggttcgcgtcagcgggtgttggcgggtgtcggggctggcttaactatgcggcatcagagcagattgtactgagagtgcaccatatgcggtgtgaaataccacacagatgcgtaaggagaaaataccgcatcaggcgccattcgccattcagctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccggggatcctctagagtcgacctgcaggcatgcaagcttgagtattctatagtctcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgtgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagcccatcgatactagcttgattgggatatctcgctcatgtttgtcgcgtgctatgtctttttaggtactttgaacctacgttcgtacttgtataatatgatcatcgtattatcgtttttcatccgtccagcgcaaaatgcattagcagctagtcctagcgtgcggagctacctggacaggtgcatgacggatgcgtgtccttcagtgactttctaattaacagtaacttctttacttatgtttcagtttgtaagaagcgggattcgctcgtcgcttgacatctgattggactgcgtcggcacgtgaaaactacattgtgaaatctgctaaaactccgggtatctctgacacaaaacgattcggcttcgcaatttcaacattacggtcaaggctaacgtatctttctcggtcaacttcagattatgccgattaaattgtcgtagctttcaaggcgttttgagtactgcggcagttgttgaacctgcaaggagaagatctcgacaacagaataaagcgaaaaatgggtctcatgcactaacactcaggcctccctcataatctctgtttgagtttaccaacaacacatatatacatttcgacaaaatgaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctgaccgacgccgaccaacaccgccggtccgacgcggcccgacgggtccgaggcctcggagatctgggcccatgcggccgcaacaactacctcgactttggctgggacactttcagtgaggacaagaagcttcagaagcgtgctatcgaactcaaccagggacgtgcggcacaaatgggcatccttgctctcatggtgcacgaacagttgggagtctctatccttccttaaaaatttaattttcattagttgcagtcactccgctttggtttctcgcgttttacgcgccgtcagtttcttggcccacgaactggattcccacttgttcatggtgtcggcttccgtccaggccttgagcagactcttttgacgagcgtttcgctgaatatcgaccttcaggtcggtcagcgcgaggcgtttgtaggaaataacttgtcgggtgactcccgtgatagattctgggccgtcaatcagacacttgttctggtcgacaatgtcaataatggtggcgagcttgccagcctccggtccgtagttgatcaaaaccacgcgacccaattcaacgtatcggtgaaacggcatggttgttaaagagattgaaaatgagagagaagtggtatgtgtatgtaaatgtgtgggtgatcctacgcaaaaacgagtttcgaagtagcagcgacacacgagatggagtgtagtttgttgttggtcaaaggacacggcgaggggcccaccacgagttgtccgtagtccgtttttgtcgggtaatttcgcatgctagagacggagcacggggttccgcgagcggcggatacccgcgtgcggtgatgcgggccgagaattttagtaaaccggtaatggagggttgaatagggtagggcgggaaaagaccaccaacctaccccggaaacgataggacatgtcgtagcggacatttaaaagaagagaatttcgttggatatacctacttcttggatttacatcacacaaccagtagaagataccgggtagagtaggtactcttcttcgacaccacacaaaacaccaaccaatacactctggaaaagacacttctaacgtgaaaggagatcatcgtccccacaatttgccgagtgatattgttcgcttcgcaacaacctagcagactctgctggtgtcacattttcggacacattttcgtctgccaaggccgcgtacacacacgcacatattcataaaactcgttcggaaatcgtttcatgtcgagttttccgtatcccgcctttctccaccaccgtccggtggacgccgcggcgcagcgtgccgcactcgcggccgttggtcgttgccgcatcgtccctacatcgtcctttcctggggccgctgactttcgcgagattgtccaaatgaacgtggcacaagcacacgtcggactcacggacgagaccgacggtgcggatcaggaaacgctccagaccacacgcaaggcccggatgaaacagggcatcgccatggccaagacgtatctcgtcgaaaccttacagttggatgtcacggtcgtggatgtggaagtcgttttggatgctcgggatgtgattcaatcggacggctgtctcgcgagtggactcctcgatgcgggttgtgacaaaattgtcgtggacggcaccaatcgggacgcttttgagactgcacaaattccgaaagcgagactgatgggccactttgtgtatccccataccgattcacgaacccgaaacgggagtctcgccgtggcctttgccgaagtccaacccttttgcgattccgtcagtgtcgagctatcgaatcctgaagccgtcacggaaagtgtcatccaggaaattgtgcaagcggcaaaagcccagcaattgcaaatttcaatcgtctacaatcccaacgactacgagaatcctggtgatcacgccgccatggttggtcgcgtatgccagagcttcccagaagggcaagtccggttggccttgaccgatccgaccgcctcgcaactcggacacgcctacgcggcgtgtctcaaaaccgaccgggacgacggtctctttaccaccgtcgtctgtactcgcaacaatgaagcactggggcttgtctactcctccacgtcgtctattgtcgccgcactggaatgtggtcggggtgtctactacagccgttcccggaacggattgtggcgcaagggcgacacgtccggacactttcaggtacttcaccggctcgacaaagactgtgacggggatgctttgcgggtgacggtcactcaaaaaggcacccctgccgcattttgccacttgaatactctaacgtgctggggtacgtcgagaggcttgcgacatttggaagaaacgctgcaagaccgactcgtgtcggctccggaaggatcctacacgaaacgtctgtttcaagacgagacactgttgcgggacaagctggtcgaagaagcgcaggaattgtcggaagccgatacgaaggcacacgtggcggaagaactggcggatgtcctctactttgccatggtgagggcggtcaaggctggagtcagtttggatgatgccgtcgcagagctagattctagggcgcgaaaagtgacgcgacgaaaaggcgatagtaaagcctttcgaattgcagcgggtgaagctattttgaaaaaagcgcagtcgtagcaacgcaagaaatagattcgtatacggaatctaggatacctccttggatattaatagcacatttctgtccttggtagaatcttgagtgttgcagcggaacgataaagggtcgaatggtcaggctgagctgaatagtccccactgagttctatccggaacggacttgcgacgaggagctgccaaagtgcaggcgtgcttccattgagtcccaacctatgctgctagcggtgtgttcgcttttagaccttgaccggggtccagacttgtctaccgaagcagggtactccgactcatccactagatgaagcggcgcatctagaaatgctccattgggaaacaaatcgtccaagctatcaaaactggatgagacactggccaacgaagtcatctcggaaatcccatcattgtattcgactgaccctccgactagccagttgcgtttgggaatttccatgtgatttggaaggcgaatcaagcgtgggaccaagaatcgatgaggagaaagcggagggagacgcctgtcatctttatgtaatcgcgggggattggtgactttgtcagcgttgatgttaccgtctgcggcgatcaaaagtgtcgcttggtcaatgtcgcttgtcaaggtcgaggtgtacgaaagttctggactatcagcaccactctgttgggcgcctcgagactggacaaagtcctctgaatttttcaaaggtaaaccaagctgtaataagttgttgcgaacggcggcctgcaagcggccgtacatagcggtcccagggagagggcttggtcgagtgggaacggcatctcgctgctgctctgtcttgttcattgtcccagatgttgccgtttgagtaaagggatgcttaattttaccacatgctgtgtctttgagctgaacaggggtgtgtttgaatgattccatgtcgctgccaccatgacatgagcaactctcgcggggctttgtagtgctccctttcccagctactcgacgcatcaggcgggcggattgatagtgaaaaccttattcattgtcagagcttaagccggtctatctttccactgtcaaacagctcttgattgtcgcccgcgcgaaaatagtagcactaactgtaacttcaaaatacaaaatgttctctgttaccatacagtgaatgtaactttcgaattgacagtattagtagtcgtattgacagtgaggcacgcccctcaatgtgcgaggtggaaaatataccagcatgacaatgaatcttggagattcttttgctgtcatcaagattcaccgccaaatcttcaggaacctatcacgtccacaggcgatgttaattcttgagtcgtcaaaacaaagtcctgtcctacctgtagaagttgacagcgagcaattgtatgcaaacttctgacttatgttataataacattaaaggtaattaagtatcttcaatttggcattttgtcactgtcagtccgttccgacaatacaggtagatttggaatgaatcttttctatgctgctgcgaatcttgtacaccttcgaggccgtagattctgtccgacgaagcgataattattgcaaaatacatggactcattattttgattcgatttctttttggtatccgactcgaaaagatccatcacggcgagcatgaatcttcgttgtatccttccgtttctcctcgcaagcttctcggctggggcttgcgtcaatctgactactcgtacccagcttccgcccgcttacaccaactctttcacccgaggcgtttactacccggacaaagttttccgttcctcggtccttcactcgacgcaggacctttttttgccgttcttttcgaacgtcacttggttccacgcgattcacgtttccggcacgaatggtacgaaacgttttgataatcccgttctgccctttaatgacggtgtctacttcgcgtccactgaaaaatcgaacatcatccgtggctggatctttggcactacgctcgattccaaaacccaatcgctgctcatcgttaataatgccaccaatgtcgttattaaagtctgcgagttccaattttgtaatgacccgtttcttggtgtgtactaccacaagaacaacaagtcgtggatggagtccgagttccgtgtgtactcctccgccaacaactgtacttttgaatacgtttcgcaaccctttcttatggatttggaaggcaaacagggcaactttaaaaatctgcgtgaatttgtcttcaagaatattgacggttacttcaaaatttactccaaacacactccgattaacttggtgcgcgatcttccccaaggtttctccgcgctcgaaccgctggtcgaccttccgatcggtatcaacattacccgcttccagactctcctggcgcttcaccgttcgtacctgactcccggtgattcgtcctcgggttggacggcgggtgcggcggcttactacgttggctaccttcaaccgcgcacgtttttgttgaagtacaacgagaacggtaccatcactgatgcggtggactgcgctttggacccgctttccgagaccaaatgtactcttaagtcctttacggtcgagaaaggtatctaccaaacttctaatttccgtgtgcagcccactgagtccatcgtgcgcttcccgaatattacgaatctttgcccctttggagaagtcttcaatgccactcgtttcgcctccgtttacgcctggaaccgtaagcgcatctccaattgcgtcgccgattactccgttctttacaactcggcttcgttttcgactttcaaatgctacggtgtgtccccgactaagctcaacgacctctgttttacgaatgtgtacgctgattcctttgtcattcgtggtgacgaagtccgccagatcgctccgggtcagaccggaaagattgcggactacaactacaaactgccggacgacttcactggatgcgtgattgcctggaattccaataacttggattcgaaagttggaggtaattacaattacttgtaccgtcttttccgcaagtcgaaccttaaaccctttgaacgtgacatttccaccgaaatttaccaagccggctcgaccccctgtaacggagtggaaggctttaattgctacttcccccttcaatcgtacggattccagccgactaacggtgttggataccaaccctaccgtgtggtggtcctgtccttcgaactgctccacgctccggcgactgtctgtggacccaagaaatcgaccaatctggtcaagaataaatgtgtcaattttaattttaacggtttgacgggaacgggtgtgctcaccgagtccaacaaaaagttccttcccttccagcagtttggtcgcgatattgcggataccacggacgcggtgcgtgatccgcaaaccctggaaattttggatattactccctgctcctttggtggtgtttccgtcattaccccgggcaccaatacttcgaatcaggttgcggtcctctaccaggacgtgaattgcactgaagtccccgtcgccattcacgccgaccagcttactccgacgtggcgcgtgtactccaccggctccaatgtttttcaaacccgtgcgggctgcctgatcggcgccgagcacgttaacaattcctacgagtgcgacatccccatcggagccggcatctgcgcttcgtaccagactcaaacgaattcgccgcgtcgcgcccgttccgtggcgtcccagtcgattattgcgtacaccatgtccctgggagcggaaaactccgttgcctactcgaataattcgattgcgatccccaccaacttcactatctcggtcactactgagattttgcccgtttccatgactaagacctccgtggattgcaccatgtacatttgcggtgactccacggaatgctccaacctgcttctgcagtacggttcgttttgcacccagttgaatcgcgcgctgaccggaattgccgtggaacaagacaaaaacactcaggaggttttcgcccaagttaagcagatctacaagacgccccccatcaaagacttcggcggattcaacttctcccagatcttgcccgatccctcgaaaccgtcgaagcgttcgtttatcgaagatctccttttcaacaaagtgacgcttgcggatgctggtttcatcaaacaatacggtgactgcttgggagacattgccgctcgtgacctcatttgcgcgcaaaagtttaatggcctcactgtgctgcccccgctgttgacggatgaaatgattgctcagtacacgtccgcgcttctggcgggaactattacgtcgggctggacgtttggtgctggcgcggcgctccaaatccccttcgcgatgcaaatggcctaccgtttcaacggtatcggagtgactcaaaacgtcttgtacgagaatcagaagctgatcgctaaccagtttaattccgcgattggtaagattcaagattcgctttcctcgacggcttccgctcttggcaagctgcaagacgtcgttaaccaaaacgctcaagctcttaacaccctggttaagcaactgtcgtccaactttggcgccatttcgtcggtgcttaacgacattctctcccgtcttgatccgccggaggctgaagtgcagatcgatcgtctcatcactggtcgtcttcagtcgcttcagacgtacgtcacccaacagcttatccgtgctgctgaaatccgtgcgtcggctaatcttgcggctacgaagatgtccgagtgcgtcctgggccagtccaagcgtgtcgacttctgtggtaaaggttaccacctcatgtcgtttccccaatcggctccgcacggtgtggtgttcctccacgtgacctacgtgccggcccaagagaaaaactttacgacggcgcccgctatttgccacgacggtaaagcgcacttcccgcgtgagggagttttcgtttccaacggcacccactggttcgtgacccaacgtaatttttacgagccgcaaatcatcactacggacaatactttcgtgtccggaaactgcgacgttgttattggcattgtcaacaataccgtgtacgatccgctgcagccggagcttgactccttcaaggaagaattggacaaatactttaagaatcacacctcgcccgatgtggacttgggcgacatctccggaattaatgcgtccgttgttaacattcagaaggagatcgaccgtctgaacgaagtggcgaagaacctcaacgaatccctgattgacctgcaagagttgggaaaatacgagcaatacattaaatggccgtggtacatctggctgggatttattgctggccttatcgctatcgtcatggtgaccattatgctctgttgcatgacttcgtgttgctcgtgtcttaagggctgctgctcctgtggatcctgctgcaaatttgacgaagacgactcggaaccggtgctgaaaggtgtcaaactgcactacactggatacattcccgaagccccccgtgacggacaggcctacgtccgtaaggacggagaatgggtcctcctctccaccttcctcgaaaacctctacttccagggacaccatcaccatcaccattgaagataagaatatctcattgtgaacatctatgatttaccaattttattctttgtttacagttagacgccagtaattgtgctgtttctctcaagtctgtgtcaatacaaactacgaaacttggcaatttttctcttgaatatgagcacgagattgaaacgcacaaaggaaattagtttccatcctttgacaaagtttgttgctgtttagagaacagatgtcaaaattaacgtgccatggaattgaaccatggtgcgctatccccaaatcacgtcgtttgacctcgtcacatttaaggtatatcaagcatattcacttatatcttgacatcctccctgcttgatattcctttgcccttgagccatcttccccacacgctggagatgacccctgcccctcctttctttatccacccgagtaaggtgtcgacaagtcactttgcccctgagccatcctccccactcaggagatgacccctacccctttcttctttatccaccccatcttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagaggcacttcgagctgtaagtacatcaccgacgagcaaggcaagacgatccgcgcctgttttattgagaacgttgttcgtgttggcctcaatggtagcgatgcgtcattcagcgaagttctggtgctgatgatgtggttcgctttgccactggtcaatgtggtaagcccgtgtaatgtcagtaacctttttactgatctcagcttgagcacggtcgctgatgagcttatccatggccccacggtaacggatatgatcctctagggcgttgacaaattctttgtcggttttcatggggtagacgtcagtgaccagggaacgtctccctacaaaaagttgcgcatactttgctccgttgtcgacggcaggggtgtcggaccagattgtatctgtggcaacggcctcattgcgtcaatggacctttaaagccgggaaacgagacttgaaatgtttacgtagaggtgcattatagacctcacgcgcatattgagtggttgcaaaaatggttttgcgaacggtatcactggagacccatgccaggcaaggacggagggcatcataatcatgttcattgcgttggacagcatgcttgttacacgtaagtatacggtcgagagcatcgtgacgttgagaattacagatggcaacgtgtcggtgcatgtattggccgagattgtcaaatcgtggctcaatatatggcatgccaggtaagtcatggatgtcggtatgccattcagccttcatgtcgatttcgttgtcgagaactgatgggtcccagtcaacgtcagaagtaagaacaacatggggaagggaatgaagttcatgactggtgggtgcgcgcatatccatgtacggaagaccctgtcgtatgtttagtgggatgatgtaatcatccagggtaacaatacgttggttgccgcccacggtacgagaccgatcttgaacacaattgtggcaatgttcaagttgggcgctggaatggatggttttacctttaccaagatgcgcgtattgatgcatgataacaacaatgggccctcgttgcgattcgacaagaccggcagccgtgacaatgtctagattggacaaggtatgttcatttataccggttatgttggctgaacgtccagttttgtgcagtacggtaacatcactccccgcaagtccaccgttggcaccgcgatcgacaagtgcagatgtggtattttgtactgtgtgtcgggagacttggtactgtaacacatttgactgaagtgacttggtggtaccatgaggacggagggaacccgttgatacggtgttgcgtgacgctgcaagtaccttacgtatgtcgcctggatccatacgtccaacccgatcagaaagatgtgctaggagttccgtttctggtgcacaatcatgaaaggtatccacgggagttccattgttgtcgtccggggctgcgtcgcgactgccagttgcatgtacatgcgccatcaagggacgtgccaatccattactaaccgggagaaccttagctggggcagccaatccctgaagtatagccttagcatcatctgagagctggttccacatatctctagggatataaggtcgttcacggttagtgggcgtactatgatgtcggttattagtacaattctccctgcgggcatgcgcattggcttcatagagtacggaaggtgacaagtcaatattgtagttaacatccggatcagtgtcaaagtctgtaggatggtaagaaagatcagtagaatgaatactacgcttttccttgggactacgggaattagagaagttgtttcctttattgtagagtgacgctgaagcaagtagaagactaaggtaactctcgtagctaataggattacctcctttggctaggtcaagagtggctgtgatcttcacttgacaaagttccggtacattgtggacagcattctccaaaagactaagacacagttgctttgggagttgctcagccattggtacagtattgtggtagatacaaaggtggttcttccaatgaaggataaatccttctgctgtgcctgtccatgaggatccatatttcgccgtagttaggtaaccaagcgtggtggctgaactgatcttcgcacttgctgattccgtatagtgtttgacaactttacaaaacacttcttgcgcagttcgctctagcctggacagagaagggacaatcttcgtcgttaagactcgtgcgaatagcaaaagatcacaaaatagcacatcggcaccgaccaacgattatttccaaggaaaaaaagaatgcttcactacaagaaattgtgtcatccctatacagagtcttgttactgtgacagaaaattgatggaagatgtggcggattgcctttacactagccaacttgttcgactaattgcagcttcttctgagaggcttcaccgagtaacgcgaagaacaccggtgtctcgtacatgctcgtcggtgaacgctcgtccaatgacaccccccactttgtatcaatatcccaacttggtagtgaactggaatgatacatgcaatttcgcgccgcatcaacagccacgggccaccatcgacgaatagactcgg

SEQ ID NO: 8 - DNA, 19487 bp, synthetic construct
  source 1..19487: mol_type = other DNA
tcgagctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttaccttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatcctagtacactctatatttttttatgcctcggtaatgattttcattttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagttttacacaggagtctggacttgacgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagcaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgacctcgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgcaaggggttcgcgtcagcgggtgttggcgggtgtcggggctggcttaactatgcggcatcagagcagattgtactgagagtgcaccatatgcggtgtgaaataccacacagatgcgtaaggagaaaataccgcatcaggcgccattcgccattcagctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccggggatcctctagagtcgacctgcaggcatgcaagcttgagtattctatagtctcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgtgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagcccatcgatactagcttgattgggatatctcgctcatgtttgtcgcgtgctatgtctttttaggtactttgaacctacgttcgtacttgtataatatgatcatcgtattatcgtttttcatccgtccagcgcaaaatgcattagcagctagtcctagcgtgcggagctacctggacaggtgcatgacggatgcgtgtccttcagtgactttctaattaacagtaacttctttacttatgtttcagtttgtaagaagcgggattcgctcgtcgcttgacatctgattggactgcgtcggcacgtgaaaactacattgtgaaatctgctaaaactccgggtatctctgacacaaaacgattcggcttcgcaatttcaacattacggtcaaggctaacgtatctttctcggtcaacttcagattatgccgattaaattgtcgtagctttcaaggcgttttgagtactgcggcagttgttgaacctgcaaggagaagatctcgacaacagaataaagcgaaaaatgggtctcatgcactaacactcaggcctccctcataatctctgtttgagtttaccaacaacacatatatacatttcgacaaaatgaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctgaccgacgccgaccaacaccgccggtccgacgcggcccgacgggtccgaggcctcggagatctgggcccatgcggccgcaacaactacctcgactttggctgggacactttcagtgaggacaagaagcttcagaagcgtgctatcgaactcaaccagggacgtgcggcacaaatgggcatccttgctctcatggtgcacgaacagttgggagtctctatccttccttaaaaatttaattttcattagttgcagtcactccgctttggtttctcgcgttttacgcgccgtcagtttcttggcccacgaactggattcccacttgttcatggtgtcggcttccgtccaggccttgagcagactcttttgacgagcgtttcgctgaatatcgaccttcaggtcggtcagcgcgaggcgtttgtaggaaataacttgtcgggtgactcccgtgatagattctgggccgtcaatcagacacttgttctggtcgacaatgtcaataatggtggcgagcttgccagcctccggtccgtagttgatcaaaaccacgcgacccaattcaacgtatcggtgaaacggcatggttgttaaagagattgaaaatgagagagaagtggtatgtgtatgtaaatgtgtgggtgatcctacgcaaaaacgagtttcgaagtagcagcgacacacgagatggagtgtagtttgttgttggtcaaaggacacggcgaggggcccaccacgagttgtccgtagtccgtttttgtcgggtaatttcgcatgctagagacggagcacggggttccgcgagcggcggatacccgcgtgcggtgatgcgggccgagaattttagtaaaccggtaatggagggttgaatagggtagggcgggaaaagaccaccaacctaccccggaaacgataggacatgtcgtagcggacatttaaaagaagagaatttcgttggatatacctacttcttggatttacatcacacaaccagtagaagataccgggtagagtaggtactcttcttcgacaccacacaaaacaccaaccaatacactctggaaaagacacttctaacgtgaaaggagatcatcgtccccacaatttgccgagtgatattgttcgcttcgcaacaacctagcagactctgctggtgtcacattttcggacacattttcgtctgccaaggccgcgtacacacacgcacatattcataaaactcgttcggaaatcgtttcatgtcgagttttccgtatcccgcctttctccaccaccgtccggtggacgccgcggcgcagcgtgccgcactcgcggccgttggtcgttgccgcatcgtccctacatcgtcctttcctggggccgctgactttcgcgagattgtccaaatgaacgtggcacaagcacacgtcggactcacggacgagaccgacggtgcggatcaggaaacgctccagaccacacgcaaggcccggatgaaacagggcatcgccatggccaagacgtatctcgtcgaaaccttacagttggatgtcacggtcgtggatgtggaagtcgttttggatgctcgggatgtgattcaatcggacggctgtctcgcgagtggactcctcgatgcgggttgtgacaaaattgtcgtggacggcaccaatcgggacgcttttgagactgcacaaattccgaaagcgagactgatgggccactttgtgtatccccataccgattcacgaacccgaaacgggagtctcgccgtggcctttgccgaagtccaacccttttgcgattccgtcagtgtcgagctatcgaatcctgaagccgtcacggaaagtgtcatccaggaaattgtgcaagcggcaaaagcccagcaattgcaaatttcaatcgtctacaatcccaacgactacgagaatcctggtgatcacgccgccatggttggtcgcgtatgccagagcttcccagaagggcaagtccggttggccttgaccgatccgaccgcctcgcaactcggacacgcctacgcggcgtgtctcaaaaccgaccgggacgacggtctctttaccaccgtcgtctgtactcgcaacaatgaagcactggggcttgtctactcctccacgtcgtctattgtcgccgcactggaatgtggtcggggtgtctactacagccgttcccggaacggattgtggcgcaagggcgacacgtccggacactttcaggtacttcaccggctcgacaaagactgtgacggggatgctttgcgggtgacggtcactcaaaaaggcacccctgccgcattttgccacttgaatactctaacgtgctggggtacgtcgagaggcttgcgacatttggaagaaacgctgcaagaccgactcgtgtcggctccggaaggatcctacacgaaacgtctgtttcaagacgagacactgttgcgggacaagctggtcgaagaagcgcaggaattgtcggaagccgatacgaaggcacacgtggcggaagaactggcggatgtcctctactttgccatggtgagggcggtcaaggctggagtcagtttggatgatgccgtcgcagagctagattctagggcgcgaaaagtgacgcgacgaaaaggcgatagtaaagcctttcgaattgcagcgggtgaagctattttgaaaaaagcgcagtcgtagcaacgcaagaaatagattcgtatacggaatctaggatacctccttggatattaatagcacatttctgtccttggtagaatcttgagtgttgcagcggaacgataaagggtcgaatggtcaggctgagctgaatagtccccactgagttctatccggaacggacttgcgacgaggagctgccaaagtgcaggcgtgcttccattgagtcccaacctatgctgctagcggtgtgttcgcttttagaccttgaccggggtccagacttgtctaccgaagcagggtactccgactcatccactagatgaagcggcgcatctagaaatgctccattgggaaacaaatcgtccaagctatcaaaactggatgagacactggccaacgaagtcatctcggaaatcccatcattgtattcgactgaccctccgactagccagttgcgtttgggaatttccatgtgatttggaaggcgaatcaagcgtgggaccaagaatcgatgaggagaaagcggagggagacgcctgtcatctttatgtaatcgcgggggattggtgactttgtcagcgttgatgttaccgtctgcggcgatcaaaagtgtcgcttggtcaatgtcgcttgtcaaggtcgaggtgtacgaaagttctggactatcagcaccactctgttgggcgcctcgagactggacaaagtcctctgaatttttcaaaggtaaaccaagctgtaataagttgttgcgaacggcggcctgcaagcggccgtacatagcggtcccagggagagggcttggtcgagtgggaacggcatctcgctgctgctctgtcttgttcattgtcccagatgttgccgtttgagtaaagggatgcttaattttaccacatgctgtgtctttgagctgaacaggggtgtgtttgaatgattccatgtcgctgccaccatgacatgagcaactctcgcggggctttgtagtgctccctttcccagctactcgacgcatcaggcgggcccctgcgatagaccttttccaaactcacgcagtccaagaaaacaaaggggtgagaagtatacgcacctttcggtttcggcataattcttaaactcttgtggtcactttcttgtgaagaagctaggggcactcgttttccctcagagcctgcaaacacaaaattcctgcagtcaattgtcccaacactcggcaaaccgtatgcgcaagcaacgatgcgcagaaggccgtggatggatggcgactcgcgatatggcttcttgggtcgccagtgtggtacgtccggcgtatgtcaatacgcgaattcggacgactggcatctctaggaggaggattccttcttttatgacatgtttattttatatacattgatgctttccgacagtcggaagtaataaatgaatttatttcaagactacctatactcctttgacttgttcgactaatcttaccgcttactaaaatctcgaaatcacgcttgacctctcgcacgaaaatttttgctgctggacgctacacactcggcccaattcttctcggtcctcgtcgtcgcaattgtcgttgcgttgatcttgcaccgaaggaatcagagaatagaataccatgttcgtgtttctggtgctgctgcctctggtgtccagccagcgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttccaccatcaccatcaccattgataaagataagaatatctcattgtgaacatctatgatttaccaattttattctttgtttacagttagacgccagtaattgtgctgtttctctcaagtctgtgtcaatacaaactacgaaacttggcaatttttctcttgaatatgagcacgagattgaaacgcacaaaggaaattagtttccatcctttgacaaagtttgttgctgtttagagaacagatgtcaaaattaacgtgccatggaattgaaccatggtgcgctatccccaaatcacgtcgtttgacctcgtcacatttaaggtatatcaagcatattcacttatatcttgacatcctccctgcttgatattcctttgcccttgagccatcttccccacacgctggagatgacccctgcccctcctttctttatccacccgagtaaggtgtcgacaagtcactttgcccctgagccatcctccccactcaggagatgacccctacccctttcttctttatccaccccatcttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagaggcacttcgagctgtaagtacatcaccgacgagcaaggcaagacgatccgcgcctgttttattgagaacgttgttcgtgttggcctcaatggtagcgatgcgtcattcagcgaagttctggtgctgatgatgtggttcgctttgccactggtcaatgtggtaagcccgtgtaatgtcagtaacctttttactgatctcagcttgagcacggtcgctgatgagcttatccatggccccacggtaacggatatgatcctctagggcgttgacaaattctttgtcggttttcatggggtagacgtcagtgaccagggaacgtctccctacaaaaagttgcgcatactttgctccgttgtcgacggcaggggtgtcggaccagattgtatctgtggcaacggcctcattgcgtcaatggacctttaaagccgggaaacgagacttgaaatgtttacgtagaggtgcattatagacctcacgcgcatattgagtggttgcaaaaatggttttgcgaacggtatcactggagacccatgccaggcaaggacggagggcatcataatcatgttcattgcgttggacagcatgcttgttacacgtaagtatacggtcgagagcatcgtgacgttgagaattacagatggcaacgtgtcggtgcatgtattggccgagattgtcaaatcgtggctcaatatatggcatgccaggtaagtcatggatgtcggtatgccattcagccttcatgtcgatttcgttgtcgagaactgatgggtcccagtcaacgtcagaagtaagaacaacatggggaagggaatgaagttcatgactggtgggtgcgcgcatatccatgtacggaagaccctgtcgtatgtttagtgggatgatgtaatcatccagggtaacaatacgttggttgccgcccacggtacgagaccgatcttgaacacaattgtggcaatgttcaagttgggcgctggaatggatggttttacctttaccaagatgcgcgtattgatgcatgataacaacaatgggccctcgttgcgattcgacaagaccggcagccgtgacaatgtctagattggacaaggtatgttcatttataccggttatgttggctgaacgtccagttttgtgcagtacggtaacatcactccccgcaagtccaccgttggcaccgcgatcgacaagtgcagatgtggtattttgtactgtgtgtcgggagacttggtactgtaacacatttgactgaagtgacttggtggtaccatgaggacggagggaacccgttgatacggtgttgcgtgacgctgcaagtaccttacgtatgtcgcctggatccatacgtccaacccgatcagaaagatgtgctaggagttccgtttctggtgcacaatcatgaaaggtatccacgggagttccattgttgtcgtccggggctgcgtcgcgactgccagttgcatgtacatgcgccatcaagggacgtgccaatccattactaaccgggagaaccttagctggggcagccaatccctgaagtatagccttagcatcatctgagagctggttccacatatctctagggatataaggtcgttcacggttagtgggcgtactatgatgtcggttattagtacaattctccctgcgggcatgcgcattggcttcatagagtacggaaggtgacaagtcaatattgtagttaacatccggatcagtgtcaaagtctgtaggatggtaagaaagatcagtagaatgaatactacgcttttccttgggactacgggaattagagaagttgtttcctttattgtagagtgacgctgaagcaagtagaagactaaggtaactctcgtagctaataggattacctcctttggctaggtcaagagtggctgtgatcttcacttgacaaagttccggtacattgtggacagcattctccaaaagactaagacacagttgctttgggagttgctcagccattggtacagtattgtggtagatacaaaggtggttcttccaatgaaggataaatccttctgctgtgcctgtccatgaggatccatatttcgccgtagttaggtaaccaagcgtggtggctgaactgatcttcgcacttgctgattccgtatagtgtttgacaactttacaaaacacttcttgcgcagttcgctctagcctggacagagaagggacaatcttcgtcgttaagactcgtgcgaatagcaaaagatcacaaaatagcacatcggcaccgaccaacgattatttccaaggaaaaaaagaatgcttcactacaagaaattgtgtcatccctatacagagtcttgttactgtgacagaaaattgatggaagatgtggcggattgcctttacactagccaacttgttcgactaattgcagcttcttctgagaggcttcaccgagtaacgcgaagaacaccggtgtctcgtacatgctcgtcggtgaacgctcgtccaatgacaccccccactttgtatcaatatcccaacttggtagtgaactggaatgatacatgcaatttcgcgccgcatcaacagccacgggccaccatcgacgaatagactcgg

SEQ ID NO: 9 - DNA, 22526 bp, synthetic construct
  source 1..22526: mol_type = other DNA
tcgagctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttaccttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatcctagtacactctatatttttttatgcctcggtaatgattttcattttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagttttacacaggagtctggacttgacgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagcaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgacctcgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgcaaggggttcgcgtcagcgggtgttggcgggtgtcggggctggcttaactatgcggcatcagagcagattgtactgagagtgcaccatatgcggtgtgaaataccacacagatgcgtaaggagaaaataccgcatcaggcgccattcgccattcagctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccggggatcctctagagtcgacctgcaggcatgcaagcttgagtattctatagtctcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgtgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagcccatcgatactagcttgattgggatatctcgctcatgtttgtcgcgtgctatgtctttttaggtactttgaacctacgttcgtacttgtataatatgatcatcgtattatcgtttttcatccgtccagcgcaaaatgcattagcagctagtcctagcgtgcggagctacctggacaggtgcatgacggatgcgtgtccttcagtgactttctaattaacagtaacttctttacttatgtttcagtttgtaagaagcgggattcgctcgtcgcttgacatctgattggactgcgtcggcacgtgaaaactacattgtgaaatctgctaaaactccgggtatctctgacacaaaacgattcggcttcgcaatttcaacattacggtcaaggctaacgtatctttctcggtcaacttcagattatgccgattaaattgtcgtagctttcaaggcgttttgagtactgcggcagttgttgaacctgcaaggagaagatctcgacaacagaataaagcgaaaaatgggtctcatgcactaacactcaggcctccctcataatctctgtttgagtttaccaacaacacatatatacatttcgacaaaatgaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctgaccgacgccgaccaacaccgccggtccgacgcggcccgacgggtccgaggcctcggagatctgggcccatgcggccgcaacaactacctcgactttggctgggacactttcagtgaggacaagaagcttcagaagcgtgctatcgaactcaaccagggacgtgcggcacaaatgggcatccttgctctcatggtgcacgaacagttgggagtctctatccttccttaaaaatttaattttcattagttgcagtcactccgctttggtttctcgcgttttacgcgccgtcagtttcttggcccacgaactggattcccacttgttcatggtgtcggcttccgtccaggccttgagcagactcttttgacgagcgtttcgctgaatatcgaccttcaggtcggtcagcgcgaggcgtttgtaggaaataacttgtcgggtgactcccgtgatagattctgggccgtcaatcagacacttgttctggtcgacaatgtcaataatggtggcgagcttgccagcctccggtccgtagttgatcaaaaccacgcgacccaattcaacgtatcggtgaaacggcatggttgttaaagagattgaaaatgagagagaagtggtatgtgtatgtaaatgtgtgggtgatcctacgcaaaaacgagtttcgaagtagcagcgacacacgagatggagtgtagtttgttgttggtcaaaggacacggcgaggggcccaccacgagttgtccgtagtccgtttttgtcgggtaatttcgcatgctagagacggagcacggggttccgcgagcggcggatacccgcgtgcggtgatgcgggccgagaattttagtaaaccggtaatggagggttgaatagggtagggcgggaaaagaccaccaacctaccccggaaacgataggacatgtcgtagcggacatttaaaagaagagaatttcgttggatatacctacttcttggatttacatcacacaaccagtagaagataccgggtagagtaggtactcttcttcgacaccacacaaaacaccaaccaatacactctggaaaagacacttctaacgtgaaaggagatcatcgtccccacaatttgccgagtgatattgttcgcttcgcaacaacctagcagactctgctggtgtcacattttcggacacattttcgtctgccaaggccgcgtacacacacgcacatattcataaaactcgttcggaaatcgtttcatgtcgagttttccgtatcccgcctttctccaccaccgtccggtggacgccgcggcgcagcgtgccgcactcgcggccgttggtcgttgccgcatcgtccctacatcgtcctttcctggggccgctgactttcgcgagattgtccaaatgaacgtggcacaagcacacgtcggactcacggacgagaccgacggtgcggatcaggaaacgctccagaccacacgcaaggcccggatgaaacagggcatcgccatggccaagacgtatctcgtcgaaaccttacagttggatgtcacggtcgtggatgtggaagtcgttttggatgctcgggatgtgattcaatcggacggctgtctcgcgagtggactcctcgatgcgggttgtgacaaaattgtcgtggacggcaccaatcgggacgcttttgagactgcacaaattccgaaagcgagactgatgggccactttgtgtatccccataccgattcacgaacccgaaacgggagtctcgccgtggcctttgccgaagtccaacccttttgcgattccgtcagtgtcgagctatcgaatcctgaagccgtcacggaaagtgtcatccaggaaattgtgcaagcggcaaaagcccagcaattgcaaatttcaatcgtctacaatcccaacgactacgagaatcctggtgatcacgccgccatggttggtcgcgtatgccagagcttcccagaagggcaagtccggttggccttgaccgatccgaccgcctcgcaactcggacacgcctacgcggcgtgtctcaaaaccgaccgggacgacggtctctttaccaccgtcgtctgtactcgcaacaatgaagcactggggcttgtctactcctccacgtcgtctattgtcgccgcactggaatgtggtcggggtgtctactacagccgttcccggaacggattgtggcgcaagggcgacacgtccggacactttcaggtacttcaccggctcgacaaagactgtgacggggatgctttgcgggtgacggtcactcaaaaaggcacccctgccgcattttgccacttgaatactctaacgtgctggggtacgtcgagaggcttgcgacatttggaagaaacgctgcaagaccgactcgtgtcggctccggaaggatcctacacgaaacgtctgtttcaagacgagacactgttgcgggacaagctggtcgaagaagcgcaggaattgtcggaagccgatacgaaggcacacgtggcggaagaactggcggatgtcctctactttgccatggtgagggcggtcaaggctggagtcagtttggatgatgccgtcgcagagctagattctagggcgcgaaaagtgacgcgacgaaaaggcgatagtaaagcctttcgaattgcagcgggtgaagctattttgaaaaaagcgcagtcgtagcaacgcaagaaatagattcgtatacggaatctaggatacctccttggatattaatagcacatttctgtccttggtagaatcttgagtgttgcagcggaacgataaagggtcgaatggtcaggctgagctgaatagtccccactgagttctatccggaacggacttgcgacgaggagctgccaaagtgcaggcgtgcttccattgagtcccaacctatgctgctagcggtgtgttcgcttttagaccttgaccggggtccagacttgtctaccgaagcagggtactccgactcatccactagatgaagcggcgcatctagaaatgctccattgggaaacaaatcgtccaagctatcaaaactggatgagacactggccaacgaagtcatctcggaaatcccatcattgtattcgactgaccctccgactagccagttgcgtttgggaatttccatgtgatttggaaggcgaatcaagcgtgggaccaagaatcgatgaggagaaagcggagggagacgcctgtcatctttatgtaatcgcgggggattggtgactttgtcagcgttgatgttaccgtctgcggcgatcaaaagtgtcgcttggtcaatgtcgcttgtcaaggtcgaggtgtacgaaagttctggactatcagcaccactctgttgggcgcctcgagactggacaaagtcctctgaatttttcaaaggtaaaccaagctgtaataagttgttgcgaacggcggcctgcaagcggccgtacatagcggtcccagggagagggcttggtcgagtgggaacggcatctcgctgctgctctgtcttgttcattgtcccagatgttgccgtttgagtaaagggatgcttaattttaccacatgctgtgtctttgagctgaacaggggtgtgtttgaatgattccatgtcgctgccaccatgacatgagcaactctcgcggggctttgtagtgctccctttcccagctactcgacgcatcaggcgggcccctgcgatagaccttttccaaactcacgcagtccaagaaaacaaaggggtgagaagtatacgcacctttcggtttcggcataattcttaaactcttgtggtcactttcttgtgaagaagctaggggcactcgttttccctcagagcctgcaaacacaaaattcctgcagtcaattgtcccaacactcggcaagccgtatgcgcaagcaacgatgcgcagaaggccgtggatggatggcgactcgcgatatggcttcttgggtcgccagtgtggtatgtccggcgtatgtcaatacgcgaattcggacgactggcatctctaggaggaggattccttcttttatgacatgtttattttatatacattgatgctttccgacagtcggaagtaataaatgaatttatttcaagactacctatactcctttgacttgttcgactaatcttaccgcttactaaaatctcgaaatcacgcttgacctctcgcacgcaaatttttgctgctggacgctacgcactcggcccaattcttctcggtcctcgtcgtcgcaattgtcgttgcgttgatcttgcaccgaaggaatcagagaatagaataccatgttcgtgtttctggtgctgctgcctctggtgtccagccagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccagcggccgcttggtcccacgtggctcacccggatctggatacatcccggaggcccctagggacggtcaagcttacgtgagaaaggacggcgaatgggttctgctgtcgaccttcttgggacatcatcatcatcatcactaatgaagataagaatatctcattgtgaacatctatgatttaccaattttattctttgtttacagttagacgccagtaattgtgctgtttctctcaagtctgtgtcaatacaaactacgaaacttggcaatttttctcttgaatatgagcacgagattgaaacgcacaaaggaaattagtttccatcctttgacaaagtttgttgctgtttagagaacagatgtcaaaattaacgtgccatggaattgaaccatggtgcgctatccccaaatcacgtcgtttgacctcgtcacatttaaggtatatcaagcatattcacttatatcttgacatcctccctgcttgatattcctttgcccttgagccatcttccccacacgctggagatgacccctgcccctcctttctttatccacccgagtaaggtgtcgacaagtcactttgcccctgagccatcctccccactcaggagatgacccctacccctttcttctttatccaccccatcttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagaggcacttcgagctgtaagtacatcaccgacgagcaaggcaagacgatccgcgcctgttttattgagaacgttgttcgtgttggcctcaatggtagcgatgcgtcattcagcgaagttctggtgctgatgatgtggttcgctttgccactggtcaatgtggtaagcccgtgtaatgtcagtaacctttttactgatctcagcttgagcacggtcgctgatgagcttatccatggccccacggtaacggatatgatcctctagggcgttgacaaattctttgtcggttttcatggggtagacgtcagtgaccagggaacgtctccctacaaaaagttgcgcatactttgctccgttgtcgacggcaggggtgtcggaccagattgtatctgtggcaacggcctcattgcgtcaatggacctttaaagccgggaaacgagacttgaaatgtttacgtagaggtgcattatagacctcacgcgcatattgagtggttgcaaaaatggttttgcgaacggtatcactggagacccatgccaggcaaggacggagggcatcataatcatgttcattgcgttggacagcatgcttgttacacgtaagtatacggtcgagagcatcgtgacgttgagaattacagatggcaacgtgtcggtgcatgtattggccgagattgtcaaatcgtggctcaatatatggcatgccaggtaagtcatggatgtcggtatgccattcagccttcatgtcgatttcgttgtcgagaactgatgggtcccagtcaacgtcagaagtaagaacaacatggggaagggaatgaagttcatgactggtgggtgcgcgcatatccatgtacggaagaccctgtcgtatgtttagtgggatgatgtaatcatccagggtaacaatacgttggttgccgcccacggtacgagaccgatcttgaacacaattgtggcaatgttcaagttgggcgctggaatggatggttttacctttaccaagatgcgcgtattgatgcatgataacaacaatgggccctcgttgcgattcgacaagaccggcagccgtgacaatgtctagattggacaaggtatgttcatttataccggttatgttggctgaacgtccagttttgtgcagtacggtaacatcactccccgcaagtccaccgttggcaccgcgatcgacaagtgcagatgtggtattttgtactgtgtgtcgggagacttggtactgtaacacatttgactgaagtgacttggtggtaccatgaggacggagggaacccgttgatacggtgttgcgtgacgctgcaagtaccttacgtatgtcgcctggatccatacgtccaacccgatcagaaagatgtgctaggagttccgtttctggtgcacaatcatgaaaggtatccacgggagttccattgttgtcgtccggggctgcgtcgcgactgccagttgcatgtacatgcgccatcaagggacgtgccaatccattactaaccgggagaaccttagctggggcagccaatccctgaagtatagccttagcatcatctgagagctggttccacatatctctagggatataaggtcgttcacggttagtgggcgtactatgatgtcggttattagtacaattctccctgcgggcatgcgcattggcttcatagagtacggaaggtgacaagtcaatattgtagttaacatccggatcagtgtcaaagtctgtaggatggtaagaaagatcagtagaatgaatactacgcttttccttgggactacgggaattagagaagttgtttcctttattgtagagtgacgctgaagcaagtagaagactaaggtaactctcgtagctaataggattacctcctttggctaggtcaagagtggctgtgatcttcacttgacaaagttccggtacattgtggacagcattctccaaaagactaagacacagttgctttgggagttgctcagccattggtacagtattgtggtagatacaaaggtggttcttccaatgaaggataaatccttctgctgtgcctgtccatgaggatccatatttcgccgtagttaggtaaccaagcgtggtggctgaactgatcttcgcacttgctgattccgtatagtgtttgacaactttacaaaacacttcttgcgcagttcgctctagcctggacagagaagggacaatcttcgtcgttaagactcgtgcgaatagcaaaagatcacaaaatagcacatcggcaccgaccaacgattatttccaaggaaaaaaagaatgcttcactacaagaaattgtgtcatccctatacagagtcttgttactgtgacagaaaattgatggaagatgtggcggattgcctttacactagccaacttgttcgactaattgcagcttcttctgagaggcttcaccgagtaacgcgaagaacaccggtgtctcgtacatgctcgtcggtgaacgctcgtccaatgacaccccccactttgtatcaatatcccaacttggtagtgaactggaatgatacatgcaatttcgcgccgcatcaacagccacgggccaccatcgacgaatagactcgg

SEQ ID NO: 10 - DNA, 759 bp, synthetic construct
  source 1..759: mol_type = other DNA
aatcttcgttgtatccttccgtttctcctcgcaagcttctcggctggggctcgtgtgcagcccactgagtccatcgtgcgcttcccgaatattacgaatctttgcccctttggagaagtcttcaatgccactcgtttcgcctccgtttacgcctggaaccgtaagcgcatctccaattgcgtcgccgattactccgttctttacaactcggcttcgttttcgactttcaaatgctacggtgtgtccccgactaagctcaacgacctctgttttacgaatgtgtacgctgattcctttgtcattcgtggtgacgaagtccgccagatcgctccgggtcagaccggaaagattgcggactacaactacaaactgccggacgacttcactggatgcgtgattgcctggaattccaataacttggattcgaaagttggaggtaattacaattacttgtaccgtcttttccgcaagtcgaaccttaaaccctttgaacgtgacatttccaccgaaatttaccaagccggctcgaccccctgtaacggagtggaaggctttaattgctacttcccccttcaatcgtacggattccagccgactaacggtgttggataccaaccctaccgtgtggtggtcctgtccttcgaactgctccacgctccggcgactgtctgtggacccaagaaatcgaccaatctggtcaagaataaatgtgtcaattttgaaaacctctacttccagggacaccatcaccatcaccat

SEQ ID NO: 11 - DNA, 642 bp, synthetic construct
  source 1..642: mol_type = other DNA
ggattgatagtgaaaaccttattcattgtcagagcttaagccggtctggtctatctttccactgtcaaacagctcttgattgtcgcccgcgcgaaaatagtagcactaactgtaacttcaaaatacaaaatgttctctgttaccatacagtgaatgtaactttcgaattgacagtattagtagtcgtattgacagtgaggcacgcccctcaatgtgcgaggtggaaaatataccagcatgacaatgaatcttggagattcttttgctgtcatcaagattcaccgccaaatcttcaggaacctatcacgtccacaggcgatgttaattcttgagtcgtcaaaacaaagtcctgtcctacctgtagaagttgacagcgagcaattgtatgcaaacttctgactttgttataataacattaaaggtaattaagtatcttcaattaggcattttgtcactgtcagtccgttccgacaatataggtagatttggaatgaatcttttctatgctgctgcgaatcttgtacacctttgaggccgtagattctgtccgacgaagcgataattattgcaaaatacatggactcattattttgattcgatttctttttggtatccgactcgaaaagatccatcacggcgagc

SEQ ID NO: 12 - DNA, 638 bp, synthetic construct
  source 1..638: mol_type = other DNA
ggattgatagtgaaaaccttattcattgtcagagcttaagccggtctatctttccactgtcaaacagctcttgattgtcgcccgcgcgaaaatagtagcactaactgtaacttcaaaatacaaaatgttctctgttaccatacagtgaatgtaactttcgaattgacagtattagtagtcgtattgacagtgaggcacgcccctcaatgtgcgaggtggaaaatataccagcatgacaatgaatcttggagattcttttgctgtcatcaagattcaccgccaaatcttcaggaacctatcacgtccacaggcgatgttaattcttgagtcgtcaaaacaaagtcctgtcctacctgtagaagttgacagcgagcaattgtatgcaaacttctgacttatgttataataacattaaaggtaattaagtatcttcaatttggcattttgtcactgtcagtccgttccgacaatacaggtagatttggaatgaatcttttctatgctgctgcgaatcttgtacaccttcgaggccgtagattctgtccgacgaagcgataattattgcaaaatacatggactcattattttgattcgatttctttttggtatccgactcgaaaagatccatcacggcgagc

SEQ ID NO: 13 - DNA, 54 bp, synthetic construct
  source 1..54: mol_type = other DNA
atgaatcttcgttgtatccttccgtttctcctcgcaagcttctcggctggggct

SEQ ID NO: 14 - DNA, 42 bp, synthetic construct
  source 1..42: mol_type = other DNA
atgttcgtgtttctggtgctgctgcctctggtgtccagccag

SEQ ID NO: 15 - DNA, 669 bp, synthetic construct
  source 1..669: mol_type = other DNA
cgtgtgcagcccactgagtccatcgtgcgcttcccgaatattacgaatctttgcccctttggagaagtcttcaatgccactcgtttcgcctccgtttacgcctggaaccgtaagcgcatctccaattgcgtcgccgattactccgttctttacaactcggcttcgttttcgactttcaaatgctacggtgtgtccccgactaagctcaacgacctctgttttacgaatgtgtacgctgattcctttgtcattcgtggtgacgaagtccgccagatcgctccgggtcagaccggaaagattgcggactacaactacaaactgccggacgacttcactggatgcgtgattgcctggaattccaataacttggattcgaaagttggaggtaattacaattacttgtaccgtcttttccgcaagtcgaaccttaaaccctttgaacgtgacatttccaccgaaatttaccaagccggctcgaccccctgtaacggagtggaaggctttaattgctacttcccccttcaatcgtacggattccagccgactaacggtgttggataccaaccctaccgtgtggtggtcctgtccttcgaactgctccacgctccggcgactgtctgtggacccaagaaatcgaccaatctggtcaagaataaatgtgtcaatttt

SEQ ID NO: 16 - DNA, 3777 bp, synthetic construct
  source 1..3777: mol_type = other DNA
tgcgtcaatctgactactcgtacccagcttccgcccgcttacaccaactctttcacccgaggcgtttactacccggacaaagttttccgttcctcggtccttcactcgacgcaggacctttttttgccgttcttttcgaacgtcacttggttccacgcgattcacgtttccggcacgaatggtacgaaacgttttgataatcccgttctgccctttaatgacggtgtctacttcgcgtccactgaaaaatcgaacatcatccgtggctggatctttggcactacgctcgattccaaaacccaatcgctgctcatcgttaataatgccaccaatgtcgttattaaagtctgcgagttccaattttgtaatgacccgtttcttggtgtgtactaccacaagaacaacaagtcgtggatggagtccgagttccgtgtgtactcctccgccaacaactgtacttttgaatacgtttcgcaaccctttcttatggatttggaaggcaaacagggcaactttaaaaatctgcgtgaatttgtcttcaagaatattgacggttacttcaaaatttactccaaacacactccgattaacttggtgcgcgatcttccccaaggtttctccgcgctcgaaccgctggtcgaccttccgatcggtatcaacattacccgcttccagactctcctggcgcttcaccgttcgtacctgactcccggtgattcgtcctcgggttggacggcgggtgcggcggcttactacgttggctaccttcaaccgcgcacgtttttgttgaagtacaacgagaacggtaccatcactgatgcggtggactgcgctttggacccgctttccgagaccaaatgtactcttaagtcctttacggtcgagaaaggtatctaccaaacttctaatttccgtgtgcagcccactgagtccatcgtgcgcttcccgaatattacgaatctttgcccctttggagaagtcttcaatgccactcgtttcgcctccgtttacgcctggaaccgtaagcgcatctccaattgcgtcgccgattactccgttctttacaactcggcttcgttttcgactttcaaatgctacggtgtgtccccgactaagctcaacgacctctgttttacgaatgtgtacgctgattcctttgtcattcgtggtgacgaagtccgccagatcgctccgggtcagaccggaaagattgcggactacaactacaaactgccggacgacttcactggatgcgtgattgcctggaattccaataacttggattcgaaagttggaggtaattacaattacttgtaccgtcttttccgcaagtcgaaccttaaaccctttgaacgtgacatttccaccgaaatttaccaagccggctcgaccccctgtaacggagtggaaggctttaattgctacttcccccttcaatcgtacggattccagccgactaacggtgttggataccaaccctaccgtgtggtggtcctgtccttcgaactgctccacgctccggcgactgtctgtggacccaagaaatcgaccaatctggtcaagaataaatgtgtcaattttaattttaacggtttgacgggaacgggtgtgctcaccgagtccaacaaaaagttccttcccttccagcagtttggtcgcgatattgcggataccacggacgcggtgcgtgatccgcaaaccctggaaattttggatattactccctgctcctttggtggtgtttccgtcattaccccgggcaccaatacttcgaatcaggttgcggtcctctaccaggacgtgaattgcactgaagtccccgtcgccattcacgccgaccagcttactccgacgtggcgcgtgtactccaccggctccaatgtttttcaaacccgtgcgggctgcctgatcggcgccgagcacgttaacaattcctacgagtgcgacatccccatcggagccggcatctgcgcttcgtaccagactcaaacgaattcgccgcgtcgcgcccgttccgtggcgtcccagtcgattattgcgtacaccatgtccctgggagcggaaaactccgttgcctactcgaataattcgattgcgatccccaccaacttcactatctcggtcactactgagattttgcccgtttccatgactaagacctccgtggattgcaccatgtacatttgcggtgactccacggaatgctccaacctgcttctgcagtacggttcgttttgcacccagttgaatcgcgcgctgaccggaattgccgtggaacaagacaaaaacactcaggaggttttcgcccaagttaagcagatctacaagacgccccccatcaaagacttcggcggattcaacttctcccagatcttgcccgatccctcgaaaccgtcgaagcgttcgtttatcgaagatctccttttcaacaaagtgacgcttgcggatgctggtttcatcaaacaatacggtgactgcttgggagacattgccgctcgtgacctcatttgcgcgcaaaagtttaatggcctcactgtgctgcccccgctgttgacggatgaaatgattgctcagtacacgtccgcgcttctggcgggaactattacgtcgggctggacgtttggtgctggcgcggcgctccaaatccccttcgcgatgcaaatggcctaccgtttcaacggtatcggagtgactcaaaacgtcttgtacgagaatcagaagctgatcgctaaccagtttaattccgcgattggtaagattcaagattcgctttcctcgacggcttccgctcttggcaagctgcaagacgtcgttaaccaaaacgctcaagctcttaacaccctggttaagcaactgtcgtccaactttggcgccatttcgtcggtgcttaacgacattctctcccgtcttgataaagtcgaggctgaagtgcagatcgatcgtctcatcactggtcgtcttcagtcgcttcagacgtacgtcacccaacagcttatccgtgctgctgaaatccgtgcgtcggctaatcttgcggctacgaagatgtccgagtgcgtcctgggccagtccaagcgtgtcgacttctgtggtaaaggttaccacctcatgtcgtttccccaatcggctccgcacggtgtggtgttcctccacgtgacctacgtgccggcccaagagaaaaactttacgacggcgcccgctatttgccacgacggtaaagcgcacttcccgcgtgagggagttttcgtttccaacggcacccactggttcgtgacccaacgtaatttttacgagccgcaaatcatcactacggacaatactttcgtgtccggaaactgcgacgttgttattggcattgtcaacaataccgtgtacgatccgctgcagccggagcttgactccttcaaggaagaattggacaaatactttaagaatcacacctcgcccgatgtggacttgggcgacatctccggaattaatgcgtccgttgttaacattcagaaggagatcgaccgtctgaacgaagtggcgaagaacctcaacgaatccctgattgacctgcaagagttgggaaaatacgagcaatacattaaatggccgtggtacatctggctgggatttattgctggccttatcgctatcgtcatggtgaccattatgctctgttgcatgacttcgtgttgctcgtgtcttaagggctgctgctcctgtggatcctgctgcaaatttgacgaagacgactcggaaccggtgctgaaaggtgtcaaactgcactacact

SEQ ID NO: 17 - DNA, 3777 bp, synthetic construct
  source 1..3777: mol_type = other DNA
tgcgtcaatctgactactcgtacccagcttccgcccgcttacaccaactctttcacccgaggcgtttactacccggacaaagttttccgttcctcggtccttcactcgacgcaggacctttttttgccgttcttttcgaacgtcacttggttccacgcgattcacgtttccggcacgaatggtacgaaacgttttgataatcccgttctgccctttaatgacggtgtctacttcgcgtccactgaaaaatcgaacatcatccgtggctggatctttggcactacgctcgattccaaaacccaatcgctgctcatcgttaataatgccaccaatgtcgttattaaagtctgcgagttccaattttgtaatgacccgtttcttggtgtgtactaccacaagaacaacaagtcgtggatggagtccgagttccgtgtgtactcctccgccaacaactgtacttttgaatacgtttcgcaaccctttcttatggatttggaaggcaaacagggcaactttaaaaatctgcgtgaatttgtcttcaagaatattgacggttacttcaaaatttactccaaacacactccgattaacttggtgcgcgatcttccccaaggtttctccgcgctcgaaccgctggtcgaccttccgatcggtatcaacattacccgcttccagactctcctggcgcttcaccgttcgtacctgactcccggtgattcgtcctcgggttggacggcgggtgcggcggcttactacgttggctaccttcaaccgcgcacgtttttgttgaagtacaacgagaacggtaccatcactgatgcggtggactgcgctttggacccgctttccgagaccaaatgtactcttaagtcctttacggtcgagaaaggtatctaccaaacttctaatttccgtgtgcagcccactgagtccatcgtgcgcttcccgaatattacgaatctttgcccctttggagaagtcttcaatgccactcgtttcgcctccgtttacgcctggaaccgtaagcgcatctccaattgcgtcgccgattactccgttctttacaactcggcttcgttttcgactttcaaatgctacggtgtgtccccgactaagctcaacgacctctgttttacgaatgtgtacgctgattcctttgtcattcgtggtgacgaagtccgccagatcgctccgggtcagaccggaaagattgcggactacaactacaaactgccggacgacttcactggatgcgtgattgcctggaattccaataacttggattcgaaagttggaggtaattacaattacttgtaccgtcttttccgcaagtcgaaccttaaaccctttgaacgtgacatttccaccgaaatttaccaagccggctcgaccccctgtaacggagtggaaggctttaattgctacttcccccttcaatcgtacggattccagccgactaacggtgttggataccaaccctaccgtgtggtggtcctgtccttcgaactgctccacgctccggcgactgtctgtggacccaagaaatcgaccaatctggtcaagaataaatgtgtcaattttaattttaacggtttgacgggaacgggtgtgctcaccgagtccaacaaaaagttccttcccttccagcagtttggtcgcgatattgcggataccacggacgcggtgcgtgatccgcaaaccctggaaattttggatattactccctgctcctttggtggtgtttccgtcattaccccgggcaccaatacttcgaatcaggttgcggtcctctaccaggacgtgaattgcactgaagtccccgtcgccattcacgccgaccagcttactccgacgtggcgcgtgtactccaccggctccaatgtttttcaaacccgtgcgggctgcctgatcggcgccgagcacgttaacaattcctacgagtgcgacatccccatcggagccggcatctgcgcttcgtaccagactcaaacgaattcgccgcgtcgcgcccgttccgtggcgtcccagtcgattattgcgtacaccatgtccctgggagcggaaaactccgttgcctactcgaataattcgattgcgatccccaccaacttcactatctcggtcactactgagattttgcccgtttccatgactaagacctccgtggattgcaccatgtacatttgcggtgactccacggaatgctccaacctgcttctgcagtacggttcgttttgcacccagttgaatcgcgcgctgaccggaattgccgtggaacaagacaaaaacactcaggaggttttcgcccaagttaagcagatctacaagacgccccccatcaaagacttcggcggattcaacttctcccagatcttgcccgatccctcgaaaccgtcgaagcgttcgtttatcgaagatctccttttcaacaaagtgacgcttgcggatgctggtttcatcaaacaatacggtgactgcttgggagacattgccgctcgtgacctcatttgcgcgcaaaagtttaatggcctcactgtgctgcccccgctgttgacggatgaaatgattgctcagtacacgtccgcgcttctggcgggaactattacgtcgggctggacgtttggtgctggcgcggcgctccaaatccccttcgcgatgcaaatggcctaccgtttcaacggtatcggagtgactcaaaacgtcttgtacgagaatcagaagctgatcgctaaccagtttaattccgcgattggtaagattcaagattcgctttcctcgacggcttccgctcttggcaagctgcaagacgtcgttaaccaaaacgctcaagctcttaacaccctggttaagcaactgtcgtccaactttggcgccatttcgtcggtgcttaacgacattctctcccgtcttgatccgccggaggctgaagtgcagatcgatcgtctcatcactggtcgtcttcagtcgcttcagacgtacgtcacccaacagcttatccgtgctgctgaaatccgtgcgtcggctaatcttgcggctacgaagatgtccgagtgcgtcctgggccagtccaagcgtgtcgacttctgtggtaaaggttaccacctcatgtcgtttccccaatcggctccgcacggtgtggtgttcctccacgtgacctacgtgccggcccaagagaaaaactttacgacggcgcccgctatttgccacgacggtaaagcgcacttcccgcgtgagggagttttcgtttccaacggcacccactggttcgtgacccaacgtaatttttacgagccgcaaatcatcactacggacaatactttcgtgtccggaaactgcgacgttgttattggcattgtcaacaataccgtgtacgatccgctgcagccggagcttgactccttcaaggaagaattggacaaatactttaagaatcacacctcgcccgatgtggacttgggcgacatctccggaattaatgcgtccgttgttaacattcagaaggagatcgaccgtctgaacgaagtggcgaagaacctcaacgaatccctgattgacctgcaagagttgggaaaatacgagcaatacattaaatggccgtggtacatctggctgggatttattgctggccttatcgctatcgtcatggtgaccattgtgctctgttgcatgacttcgtgttgctcgtgtcttaagggctgctgctcctgtggatcctgctgcaaatttgacgaagacgactcggaaccggtgctgaaaggtgtcaaactgcactacact

SEQ ID NO: 18 - DNA, 669 bp, synthetic construct
  source 1..669: mol_type = other DNA
cgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttc

SEQ ID NO: 19 - DNA, 2877 bp, synthetic construct
  source 1..2877: mol_type = other DNA
cgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagccccgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggacaaggtggaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccagcggccgcttggtcccacgtggctcacccggatctggatacatcccggaggcccctagggacggtcaagcttacgtgagaaaggacggcgaatgggttctgctgtcgaccttcttggga

SEQ ID NO: 20 - DNA, 687 bp, synthetic construct
  source 1..687: mol_type = other DNA
cgtgtgcagcccactgagtccatcgtgcgcttcccgaatattacgaatctttgcccctttggagaagtcttcaatgccactcgtttcgcctccgtttacgcctggaaccgtaagcgcatctccaattgcgtcgccgattactccgttctttacaactcggcttcgttttcgactttcaaatgctacggtgtgtccccgactaagctcaacgacctctgttttacgaatgtgtacgctgattcctttgtcattcgtggtgacgaagtccgccagatcgctccgggtcagaccggaaagattgcggactacaactacaaactgccggacgacttcactggatgcgtgattgcctggaattccaataacttggattcgaaagttggaggtaattacaattacttgtaccgtcttttccgcaagtcgaaccttaaaccctttgaacgtgacatttccaccgaaatttaccaagccggctcgaccccctgtaacggagtggaaggctttaattgctacttcccccttcaatcgtacggattccagccgactaacggtgttggataccaaccctaccgtgtggtggtcctgtccttcgaactgctccacgctccggcgactgtctgtggacccaagaaatcgaccaatctggtcaagaataaatgtgtcaattttcaccatcaccatcaccat

SEQ ID NO: 21 - DNA, 20144 bp, synthetic construct
  source 1..20144: mol_type = other DNA
tcgagctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttaccttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatcctagtacactctatatttttttatgcctcggtaatgattttcattttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagttttacacaggagtctggacttgacgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagcaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgacctcgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgcaaggggttcgcgtcagcgggtgttggcgggtgtcggggctggcttaactatgcggcatcagagcagattgtactgagagtgcaccatatgcggtgtgaaataccacacagatgcgtaaggagaaaataccgcatcaggcgccattcgccattcagctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccggggatcctctagagtcgacctgcaggcatgcaagcttgagtattctatagtctcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcgacggaagtctacacgaaggtttttgcgctggatgtggctgcccggcaccgggtgcagtttgtgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagcccatcgatactagcttgattgggatatctcgctcatgtttgtcgcgtgctatgtctttttaggtactttgaacctacgttcgtacttgtataatatgatcatcgtattatcgtttttcatccgtccagcgcaaaatgcattagcagctagtcctagcgtgcggagctacctggacaggtgcatgacggatgcgtgtccttcagtgactttctaattaacagtaacttctttacttatgtttcagtttgtaagaagcgggattcgctcgtcgcttgacatctgattggactgcgtcggcacgtgaaaactacattgtgaaatctgctaaaactccgggtatctctgacacaaaacgattcggcttcgcaatttcaacattacggtcaaggctaacgtatctttctcggtcaacttcagattatgccgattaaattgtcgtagctttcaaggcgttttgagtactgcggcagttgttgaacctgcaaggagaagatctcgacaacagaataaagcgaaaaatgggtctcatgcactaacactcaggcctccctcataatctctgtttgagtttaccaacaacacatatatacatttcgacaaaatgaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctgaccgacgccgaccaacaccgccggtccgacgcggcccgacgggtccgaggcctcggagatctgggcccatgcggccgcaacaactacctcgactttggctgggacactttcagtgaggacaagaagcttcagaagcgtgctatcgaactcaaccagggacgtgcggcacaaatgggcatccttgctctcatggtgcacgaacagttgggagtctctatccttccttaaaaatttaattttcattagttgcagtcactccgctttggtttctcgcgttttacgcgccgtcagtttcttggcccacgaactggattcccacttgttcatggtgtcggcttccgtccaggccttgagcagactcttttgacgagcgtttcgctgaatatcgaccttcaggtcggtcagcgcgaggcgtttgtaggaaataacttgtcgggtgactcccgtgatagattctgggccgtcaatcagacacttgttctggtcgacaatgtcaataatggtggcgagcttgccagcctccggtccgtagttgatcaaaaccacgcgacccaattcaacgtatcggtgaaacggcatggttgttaaagagattgaaaatgagagagaagtggtatgtgtatgtaaatgtgtgggtgatcctacgcaaaaacgagtttcgaagtagcagcgacacacgagatggagtgtagtttgttgttggtcaaaggacacggcgaggggcccaccacgagttgtccgtagtccgtttttgtcgggtaatttcgcatgctagagacggagcacggggttccgcgagcggcggatacccgcgtgcggtgatgcgggccgagaattttagtaaaccggtaatggagggttgaatagggtagggcgggaaaagaccaccaacctaccccggaaacgataggacatgtcgtagcggacatttaaaagaagagaatttcgttggatatacctacttcttggatttacatcacacaaccagtagaagataccgggtagagtaggtactcttcttcgacaccacacaaaacaccaaccaatacactctggaaaagacacttctaacgtgaaaggagatcatcgtccccacaatttgccgagtgatattgttcgcttcgcaacaacctagcagactctgctggtgtcacattttcggacacattttcgtctgccaaggccgcgtacacacacgcacatattcataaaactcgttcggaaatcgtttcatgtcgagttttccgtatcccgcctttctccaccaccgtccggtggacgccgcggcgcagcgtgccgcactcgcggccgttggtcgttgccgcatcgtccctacatcgtcctttcctggggccgctgactttcgcgagattgtccaaatgaacgtggcacaagcacacgtcggactcacggacgagaccgacggtgcggatcaggaaacgctccagaccacacgcaaggcccggatgaaacagggcatcgccatggccaagacgtatctcgtcgaaaccttacagttggatgtcacggtcgtggatgtggaagtcgttttggatgctcgggatgtgattcaatcggacggctgtctcgcgagtggactcctcgatgcgggttgtgacaaaattgtcgtggacggcaccaatcgggacgcttttgagactgcacaaattccgaaagcgagactgatgggccactttgtgtatccccataccgattcacgaacccgaaacgggagtctcgccgtggcctttgccgaagtccaacccttttgcgattccgtcagtgtcgagctatcgaatcctgaagccgtcacggaaagtgtcatccaggaaattgtgcaagcggcaaaagcccagcaattgcaaatttcaatcgtctacaatcccaacgactacgagaatcctggtgatcacgccgccatggttggtcgcgtatgccagagcttcccagaagggcaagtccggttggccttgaccgatccgaccgcctcgcaactcggacacgcctacgcggcgtgtctcaaaaccgaccgggacgacggtctctttaccaccgtcgtctgtactcgcaacaatgaagcactggggcttgtctactcctccacgtcgtctattgtcgccgcactggaatgtggtcggggtgtctactacagccgttcccggaacggattgtggcgcaagggcgacacgtccggacactttcaggtacttcaccggctcgacaaagactgtgacggggatgctttgcgggtgacggtcactcaaaaaggcacccctgccgcattttgccacttgaatactctaacgtgctggggtacgtcgagaggcttgcgacatttggaagaaacgctgcaagaccgactcgtgtcggctccggaaggatcctacacgaaacgtctgtttcaagacgagacactgttgcgggacaagctggtcgaagaagcgcaggaattgtcggaagccgatacgaaggcacacgtggcggaagaactggcggatgtcctctactttgccatggtgagggcggtcaaggctggagtcagtttggatgatgccgtcgcagagctagattctagggcgcgaaaagtgacgcgacgaaaaggcgatagtaaagcctttcgaattgcagcgggtgaagctattttgaaaaaagcgcagtcgtagcaacgcaagaaatagattcgtatacggaatctaggatacctccttggatattaatagcacatttctgtccttggtagaatcttgagtgttgcagcggaacgataaagggtcgaatggtcaggctgagctgaatagtccccactgagttctatccggaacggacttgcgacgaggagctgccaaagtgcaggcgtgcttccattgagtcccaacctatgctgctagcggtgtgttcgcttttagaccttgaccggggtccagacttgtctaccgaagcagggtactccgactcatccactagatgaagcggcgcatctagaaatgctccattgggaaacaaatcgtccaagctatcaaaactggatgagacactggccaacgaagtcatctcggaaatcccatcattgtattcgactgaccctccgactagccagttgcgtttgggaatttccatgtgatttggaaggcgaatcaagcgtgggaccaagaatcgatgaggagaaagcggagggagacgcctgtcatctttatgtaatcgcgggggattggtgactttgtcagcgttgatgttaccgtctgcggcgatcaaaagtgtcgcttggtcaatgtcgcttgtcaaggtcgaggtgtacgaaagttctggactatcagcaccactctgttgggcgcctcgagactggacaaagtcctctgaatttttcaaaggtaaaccaagctgtaataagttgttgcgaacggcggcctgcaagcggccgtacatagcggtcccagggagagggcttggtcgagtgggaacggcatctcgctgctgctctgtcttgttcattgtcccagatgttgccgtttgagtaaagggatgcttaattttaccacatgctgtgtctttgagctgaacaggggtgtgtttgaatgattccatgtcgctgccaccatgacatgagcaactctcgcggggctttgtagtgctccctttcccagctactcgacgcatcaggcgggcggattgatagtgaaaaccttattcattgtcagagcttaagccggtctggtctatctttccactgtcaaacagctcttgattgtcgcccgcgcgaaaatagtagcactaactgtaacttcaaaatacaaaatgttctctgttaccatacagtgaatgtaactttcgaattgacagtattagtagtcgtattgacagtgaggcacgcccctcaatgtgcgaggtggaaaatataccagcatgacaatgaatcttggagattcttttgctgtcatcaagattcaccgccaaatcttcaggaacctatcacgtccacaggcgatgttaattcttgagtcgtcaaaacaaagtcctgtcctacctgtagaagttgacagcgagcaattgtatgcaaacttctgactttgttataataacattaaaggtaattaagtatcttcaattaggcattttgtcactgtcagtccgttccgacaatataggtagatttggaatgaatcttttctatgctgctgcgaatcttgtacacctttgaggccgtagattctgtccgacgaagcgataattattgcaaaatacatggactcattattttgattcgatttctttttggtatccgactcgaaaagatccatcacggcgagcatgaatcttcgttgtatccttccgtttctcctcgcaagcttctcggctggggcttcggataacggtccccagaaccagcgtaatgctccgcgtattacttttggaggtccgtccgactcgaccggatcgaaccaaaacggtgagcgttccggcgcgcgttcgaaacagcgccgtccgcagggtctccccaataacactgcttcgtggtttactgctctcactcaacacggcaaagaagacttgaaatttccccgtggtcagggcgttccgatcaacactaactcgtcccccgacgatcagatcggatactaccgccgtgctacccgtcgcatccgcggtggagacggcaaaatgaaagatttgtccccgcgctggtacttctactacttgggtaccggacccgaagctggcctgccgtacggtgccaacaaagatggcatcatttgggtcgcgactgagggtgctctcaacactccgaaagatcacatcggaacgcgcaacccggccaataacgcggcgattgttttgcaactcccccaaggcaccaccctcccgaaaggtttttacgcggaaggctcccgtggtggatcgcaagcctcctcccgttcctcctcgcgctcccgtaactcctcccgtaattccacgcccggctcctcgaagcgcacttcgccggctcgtatggctggtaacggcggcgacgcggctctggctctcttgctcttggaccgtctgaatcagttggagtcgaaaatgtccggcaaaggccaacaacagcaaggacaaacggtgaccaaaaaatccgctgctgaagcgtcgaaaaaaccgcgccagaaacgcaccgctaccaaggcttacaacgttactcaggcttttggccgtcgcggccccgaacagactcaaggcaattttggagatcaggagttgatccgtcaaggtacggactacaagcactggccccagatcgcccaatttgctccgtccgcgtcggcgttctttggcatgtcgcgcattggtatggaagtcaccccgtccggcacttggctgacctacaccggtgctattaagctcgatgacaaggaccccaactttaaggaccaagttatcttgttgaataagcacatcgacgcctacaagacgtttccgcccactgaacccaagaaggacaaaaagaagaaagctgacgagacccaggcgctcccccagcgccaaaagaaacaacagaccgttacgctcttgccggctgctgatttggatgacttctccaaacagttgcagcagtccatgtcgtcggctgattcgactcaagccgaaaacctctacttccagggacaccatcaccatcaccattgaagataagaatatctcattgtgaacatctatgatttaccaattttattctttgtttacagttagacgccagtaattgtgctgtttctctcaagtctgtgtcaatacaaactacgaaacttggcaatttttctcttgaatatgagcacgagattgaaacgcacaaaggaaattagtttccatcctttgacaaagtttgttgctgtttagagaacagatgtcaaaattaacgtgccatggaattgaaccatggtgcgctatccccaaatcacgtcgtttgacctcgtcacatttaaggtatatcaagcatattcacttatatcttgacatcctccctgcttgatattcctttgcccttgagccatcttccccacacgctggagatgacccctgcccctcctttctttatccacccgagtaaggtgtcgacaagtcactttgcccctgagccatcctccccactcaggagatgacccctacccctttcttctttatccaccccatcttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagaggcacttcgagctgtaagtacatcaccgacgagcaaggcaagacgatccgcgcctgttttattgagaacgttgttcgtgttggcctcaatggtagcgatgcgtcattcagcgaagttctggtgctgatgatgtggttcgctttgccactggtcaatgtggtaagcccgtgtaatgtcagtaacctttttactgatctcagcttgagcacggtcgctgatgagcttatccatggccccacggtaacggatatgatcctctagggcgttgacaaattctttgtcggttttcatggggtagacgtcagtgaccagggaacgtctccctacaaaaagttgcgcatactttgctccgttgtcgacggcaggggtgtcggaccagattgtatctgtggcaacggcctcattgcgtcaatggacctttaaagccgggaaacgagacttgaaatgtttacgtagaggtgcattatagacctcacgcgcatattgagtggttgcaaaaatggttttgcgaacggtatcactggagacccatgccaggcaaggacggagggcatcataatcatgttcattgcgttggacagcatgcttgttacacgtaagtatacggtcgagagcatcgtgacgttgagaattacagatggcaacgtgtcggtgcatgtattggccgagattgtcaaatcgtggctcaatatatggcatgccaggtaagtcatggatgtcggtatgccattcagccttcatgtcgatttcgttgtcgagaactgatgggtcccagtcaacgtcagaagtaagaacaacatggggaagggaatgaagttcatgactggtgggtgcgcgcatatccatgtacggaagaccctgtcgtatgtttagtgggatgatgtaatcatccagggtaacaatacgttggttgccgcccacggtacgagaccgatcttgaacacaattgtggcaatgttcaagttgggcgctggaatggatggttttacctttaccaagatgcgcgtattgatgcatgataacaacaatgggccctcgttgcgattcgacaagaccggcagccgtgacaatgtctagattggacaaggtatgttcatttataccggttatgttggctgaacgtccagttttgtgcagtacggtaacatcactccccgcaagtccaccgttggcaccgcgatcgacaagtgcagatgtggtattttgtactgtgtgtcgggagacttggtactgtaacacatttgactgaagtgacttggtggtaccatgaggacggagggaacccgttgatacggtgttgcgtgacgctgcaagtaccttacgtatgtcgcctggatccatacgtccaacccgatcagaaagatgtgctaggagttccgtttctggtgcacaatcatgaaaggtatccacgggagttccattgttgtcgtccggggctgcgtcgcgactgccagttgcatgtacatgcgccatcaagggacgtgccaatccattactaaccgggagaaccttagctggggcagccaatccctgaagtatagccttagcatcatctgagagctggttccacatatctctagggatataaggtcgttcacggttagtgggcgtactatgatgtcggttattagtacaattctccctgcgggcatgcgcattggcttcatagagtacggaaggtgacaagtcaatattgtagttaacatccggatcagtgtcaaagtctgtaggatggtaagaaagatcagtagaatgaatactacgcttttccttgggactacgggaattagagaagttgtttcctttattgtagagtgacgctgaagcaagtagaagactaaggtaactctcgtagctaataggattacctcctttggctaggtcaagagtggctgtgatcttcacttgacaaagttccggtacattgtggacagcattctccaaaagactaagacacagttgctttgggagttgctcagccattggtacagtattgtggtagatacaaaggtggttcttccaatgaaggataaatccttctgctgtgcctgtccatgaggatccatatttcgccgtagttaggtaaccaagcgtggtggctgaactgatcttcgcacttgctgattccgtatagtgtttgacaactttacaaaacacttcttgcgcagttcgctctagcctggacagagaagggacaatcttcgtcgttaagactcgtgcgaatagcaaaagatcacaaaatagcacatcggcaccgaccaacgattatttccaaggaaaaaaagaatgcttcactacaagaaattgtgtcatccctatacagagtcttgttactgtgacagaaaattgatggaagatgtggcggattgcctttacactagccaacttgttcgactaattgcagcttcttctgagaggcttcaccgagtaacgcgaagaacaccggtgtctcgtacatgctcgtcggtgaacgctcgtccaatgacaccccccactttgtatcaatatcccaacttggtagtgaactggaatgatacatgcaatttcgcgccgcatcaacagccacgggccaccatcgacgaatagactcgg

SEQ ID NO: 22 - DNA, 1329 bp, synthetic construct
  source 1..1329: mol_type = other DNA
atgaatcttcgttgtatccttccgtttctcctcgcaagcttctcggctggggcttcggataacggtccccagaaccagcgtaatgctccgcgtattacttttggaggtccgtccgactcgaccggatcgaaccaaaacggtgagcgttccggcgcgcgttcgaaacagcgccgtccgcagggtctccccaataacactgcttcgtggtttactgctctcactcaacacggcaaagaagacttgaaatttccccgtggtcagggcgttccgatcaacactaactcgtcccccgacgatcagatcggatactaccgccgtgctacccgtcgcatccgcggtggagacggcaaaatgaaagatttgtccccgcgctggtacttctactacttgggtaccggacccgaagctggcctgccgtacggtgccaacaaagatggcatcatttgggtcgcgactgagggtgctctcaacactccgaaagatcacatcggaacgcgcaacccggccaataacgcggcgattgttttgcaactcccccaaggcaccaccctcccgaaaggtttttacgcggaaggctcccgtggtggatcgcaagcctcctcccgttcctcctcgcgctcccgtaactcctcccgtaattccacgcccggctcctcgaagcgcacttcgccggctcgtatggctggtaacggcggcgacgcggctctggctctcttgctcttggaccgtctgaatcagttggagtcgaaaatgtccggcaaaggccaacaacagcaaggacaaacggtgaccaaaaaatccgctgctgaagcgtcgaaaaaaccgcgccagaaacgcaccgctaccaaggcttacaacgttactcaggcttttggccgtcgcggccccgaacagactcaaggcaattttggagatcaggagttgatccgtcaaggtacggactacaagcactggccccagatcgcccaatttgctccgtccgcgtcggcgttctttggcatgtcgcgcattggtatggaagtcaccccgtccggcacttggctgacctacaccggtgctattaagctcgatgacaaggaccccaactttaaggaccaagttatcttgttgaataagcacatcgacgcctacaagacgtttccgcccactgaacccaagaaggacaaaaagaagaaagctgacgagacccaggcgctcccccagcgccaaaagaaacaacagaccgttacgctcttgccggctgctgatttggatgacttctccaaacagttgcagcagtccatgtcgtcggctgattcgactcaagccgaaaacctctacttccaggga

SEQ ID NO: 23 - protein, 443 aa, synthetic construct
MNLRCILPFLLASFSAGASDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSKRTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAENLYFQG